(19) United States
(12) Patent Application Publication
(10) Pub. No.: US 2011/0319396 A1
(43) Pub. Date: Dec. 29, 2011

(54) BENZODIAZEPIN-2-ON DERIVATIVES

(75) Inventors: Masanori Asai, Ibaraki (JP); Tasuku Haketa, Hyogo (JP); Seiichi Inamura, Kanagawa (JP); Makoto Ishikawa, Shizuoka (JP); Hideki Jona, Ibaraki (JP); Hiroshi Kawamoto, Ibaraki (JP); Hideki Kurihara, Kanagawa (JP); Jun Shibata, Tokyo (JP); Tadashi Shimamura, Ibaraki (JP); Takuya Suga, Ibaraki (JP); Hitomi Watanabe, Ibaraki (JP)

(73) Assignee: MSD K.K., Chiyoda-ku (JP)

(21) Appl. No.: 13/142,005

(22) PCT Filed: Jan. 19, 2010
(86) PCT No.: PCT/JP2010/050867

§ 371 Date: Jun. 24, 2011

(30) Foreign Application Priority Data
Jan. 23, 2009 (JP) 2009-013177

(51) Int. Cl. A61K 31/5513; C07D 401/12; C07D 417/12; A61P 3/04; C07D 409/12; A61P 3/06; A61P 3/10; C07D 243/14; C07D 403/12

(52) U.S. Cl. 514/221; 540/509

(57) ABSTRACT

The present invention relates to a compound represented by the formula (I): wherein R¹ represents a hydrogen atom or the like; R² represents lower alkyl or the like; R³ and R⁴ represent lower alkyl or the like; R⁵ represents phenyl or the like; R⁶ represents a hydrogen atom or the like; m is an integer of from 0 to 2; p is an integer of from 1 to 4; and q is an integer of from 1 to 5, or a pharmaceutical acceptable salt thereof, and a DGAT 1 inhibitor comprising the compound.

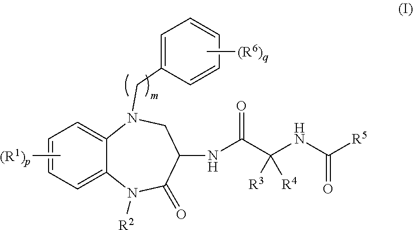

### TECHNICAL FIELD

The present invention relates to benzodiazepin-2-on derivatives which are useful in the pharmaceutical field. These compounds have inhibitory activity of diacylglycerol O-acyltransferase type 1 (hereinafter also referred to as “DGAT1”) and are useful as agents for treating and/or preventing hyperlipidemia, diabetes and obesity.

### BACKGROUND ART

Obesity is a condition, in which the background of lack of exercise, intake of excessive energy, ageing, etc. leads to energy imbalance, the surplus energy is accumulated generally as neutral fat (triacylglycerol, TG) in adipose tissue, and body weight and fat mass are thus increased. In recent years, the concept of metabolic syndrome associated with obesity involving the accumulation of the visceral fat, as an upstream risk factor including a plurality of risk factors of diabetes, lipidosis, hypertension, etc. has been established, and the diagnostic criteria and therapeutic guidelines for the metabolic syndrome were formulated (Journal of Japan Society for the Study of Obesity, Vol. 12, Extra Edition, 2006). Since the metabolic syndrome results in increase in the risks of arteriosclerosis, cardiovascular disorder and cerebrovascular disorder, treatment of obesity has been recognized to be important for preventing these diseases.

Although the need of treating obesity is recognized to be important, there are extremely-limited drug therapies for obesity that are currently available, and the advent of novel antiobestic drugs having more definite action and few side-effects is thus desired.

In the living body, there are two TG synthesis pathways of a glycerol phosphate pathway, which is present in most organs and causes de novo TG synthesis, and a monoacylglycerol pathway, which is involved principally in absorption of aliphatic acid from the small intestine. Diacylglycerol acyltransferases (DGATs, EC 2.3.1.20), which are membrane-bound enzymes present in the endoplasmic reticulum, catalyze the final step of the TG synthesis common to the two TG synthesis pathways, that is, the reaction of transferring an acyl group of acyl-coenzyme A to the 3-position of 1,2-diacylglycerol to generate TG (Prog. Lipid Res., 43, 134-176, 2004; Ann. Med., 36, 252-261, 2004).

DGATs have been found to include two subtypes of DGATs 1 and 2. There is no significant homology at the generic or amino acid level between the DGATs 1 and 2, which are encoded by different genes (Proc. Natl. Acad. Sci. USA., 95, 13018-13023, 1998; JBC, 276, 38870-38876, 2001). DGAT1, which is present in the small intestine, adipose tissue, the liver, etc., is believed to be involved in lipid absorption; lipid accumulation in the fat cell; and VLDL secretion and lipid accumulation in the liver, in the small intestine, the fat cell and the liver, respectively (Ann Med., 36, 252-261, 2004; JBC, 280, 21506-21514, 2005). In consideration of these functions of DGAT1, a DGAT1 inhibitor is expected to improve metabolic syndrome through inhibition of the lipid absorption in the small intestine, the lipid accumulation in the adipose tissue and the liver, and the lipid secretion from the liver.

In order to carry out in vivo examination of the physiological function(s) of DGAT1 and inhibitory activity against DGAT 1, DGAT1-knockout mice deficient in DGAT1 at the generic level were produced, and analyses thereof were conducted. As a result, the DGAT1-knockout mice have been found to have smaller fat masses than those of wild-type mice and to exhibit resistance to obesity, abnormal glucose tolerance, insulin resistance and fatty liver due to a high-fat diet load (Nature Genetics, 25, 87-90, 2000; JCI, 109, 1049-1055, 2002). In addition, energy expense has been reported to be accelerated in the DGAT1-knockout mice; and transplantation of the adipose tissues of DGAT1-knockout mice into wild-type mice has been reported to make the wild-type mice resistant to obesity and abnormal glucose tolerance, induced by a high-fat diet load (JCI, 111, 1715-1722, 2003; Diabetes, 53, 1445-1451, 2004). In contrast, obesity and diabetes due to a high-fat diet load have been reported to worsen in mice with overexpression of DGAT1 in adipose tissue (Diabetes, 51, 3189-3195, 2002; Diabetes, 54, 3379-3386).

From the results, DGAT1 inhibitors are likely to be therapeutic drugs with efficacy for obesity or type 2 diabetes, lipidosis, hypertension, fatty liver, arteriosclerosis, cerebrovascular disorder, coronary artery disease, or the like, associated with the obesity.

Some compounds having DGAT1 inhibitory activity have been known, all of which have different structures from that of a compound according to an embodiment of the present invention (for example, see WO 2004/100881, WO 2006/044775 and WO 2006/113919).

Also, benzodiazepin-2-on derivatives are disclosed in WO 99/66934. The benzodiazepine derivatives disclosed in the document have structures different from that of the compound according to an embodiment of the invention. Furthermore, the document does not disclose or suggest that the compounds have DGAT1 inhibitory action and are also useful in treatment and/or prevention of hyperlipidemia, diabetes and obesity.

### DISCLOSURE OF THE INVENTION

It is desirable to provide benzodiazepin-2-on derivatives having DGAT1 inhibitory activity.

The present inventors have conducted extensive research for developing a compound having DGAT1 inhibitory activity. They found that a compound according to an embodiment of the present invention is efficacious as a compound having DGAT1 inhibitory activity.

Specifically, the present invention relates to an agent for treating and/or preventing hyperlipidemia, diabetes and obesity, which contains a compound represented by the formula (I):

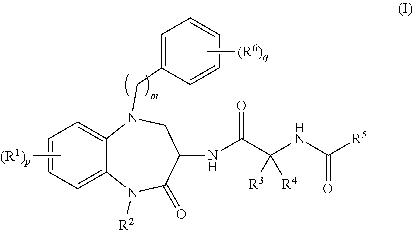

wherein R¹ each independently represents a hydrogen or halogen atom;
R² represents a hydrogen atom or lower alkyl;
R³ and R⁴ each independently represent lower alkyl or represent C₃₋₇ cycloalkyl formed by R³ and R⁴ together with the carbon atom to which they are bound;
R⁵ is a group selected from the group consisting of:
(1) phenyl, which may be substituted with 1 to 3 same or different groups selected from the group consisting of halogen atoms and lower alkoxy which may be substituted with 1 to 3 same or different halogen atoms;
(2) heteroaryl selected from the group consisting of pyridinyl, pyrazinyl, imidazolyl, thiazolyl, thienyl and oxazolyl, which heteroaryl may be substituted with 1 to 3 same or different groups selected from the group consisting of halogen atoms and lower alkyl which may be substituted with 1 to 3 same or different halogen atoms;
(3) —O—C₃₋₆ branched lower alkyl, which may be substituted with 1 to 3 same or different halogen atoms;
(4) C₃₋₇ cycloalkyl, which may be substituted with trifluoromethyl; and
(5) —N—C₃₋₆ branched lower alkyl or —N(R⁷)R⁸, wherein N, R⁷ and R⁸ together form a 5-7 membered ring;
R⁶ each independently represents a group selected from the group consisting of a hydrogen atom, lower alkyl, lower alkoxy, a halogen atom, cyano and lower alkoxycarbonylmethyl;
m is an integer from 0 to 2;
p is an integer from 1 to 4; and
q is an integer from 1 to 5,
or a pharmaceutically acceptable salt thereof as an active ingredient.

The present invention also relates to a pharmaceutical composition containing the compound represented by the formula (I) and a pharmaceutically acceptable carrier.

The present invention also relates to a DGAT1 inhibitor containing the compound represented by the formula (I) or a pharmaceutically acceptable salt thereof as an active ingredient.

The present invention also relates to an agent for treating and/or preventing hyperlipidemia, diabetes and obesity, which contains the compound represented by the formula (I) or a pharmaceutically acceptable salt thereof as an active ingredient.

The present invention further relates to a pharmaceutical composition containing the compound represented by the formula (I) and a pharmaceutically acceptable carrier.

A compound (1) according to an embodiment of the present invention or a pharmaceutically acceptable salt thereof has strong DGAT1 inhibitory activity and is thus useful for treating and/or preventing hyperlipidemia, diabetes and obesity.

The meanings of terms as used herein are described below, and a compound according to an embodiment of the present invention is described in further detail.

The term “halogen atom” encompasses, for example, fluorine, chlorine, bromine and iodine atoms.

The term “lower alkyl” refers to linear or branched C₁₋₆ alkyl, examples of which include methyl, ethyl, propyl, isopropyl, butyl, isobutyl, sec-butyl, tert-butyl, pentyl, isoamyl, neopentyl, isopentyl, 1,1-dimethylpropyl, 1-methyl butyl, 2-methyl butyl, 1,2-dimethylpropyl, hexyl, isohexyl, 1-methyl pentyl, 2-methyl pentyl, 3-methyl pentyl, 1,1-dimethyl butyl, 1,2-dimethyl butyl, 2,2-dimethyl butyl, 1,3-dimethyl butyl, 2,3-dimethyl butyl, 3,3-dimethyl butyl, 1-ethyl butyl, 2-ethyl butyl, 1,2,2-trimethylpropyl and 1-ethyl-2-methylpropyl.

The term “lower alkoxy” refers to a group in which the hydrogen atom of hydroxy is substituted with the above-mentioned lower alkyl, examples of which include methoxy, ethoxy, propoxy, isopropoxy, butoxy, sec-butoxy, tert-butoxy, pentyloxy, isopentyloxy, hexyloxy and isohexyloxy.

The term “C₃₋₇ cycloalkyl” specifically encompasses cyclopropyl, cyclobutyl, cyclopentyl, cyclohexyl and cycloheptyl.

In order to further disclose a compound according to an embodiment of the present invention, represented by the formula (I):

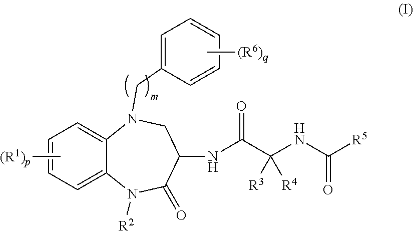

wherein each symbol has the same definition specified above, each symbol used in the formula (I) is described referring to specific examples.

R¹ each independently represents a hydrogen or halogen atom.

“Halogen atom” represented by R¹ encompasses same groups as the halogen atoms defined above, of which examples specifically include fluorine, chlorine, bromine and iodine atoms.

R¹ is preferably a hydrogen, chlorine or fluorine atom.

R² represents a hydrogen atom or lower alkyl.

“Lower alkyl” represented by R² refers to a same group as the lower alkyl defined above, of which examples specifically include methyl, ethyl and isopropyl.

R² is preferably methyl.

R³ and R⁴ each independently represent lower alkyl or represent C₃₋₇ cycloalkyl formed by R³ and R⁴ together with the carbon atom to which they are bound, except in cases where both R³ and R⁴ are hydrogen atoms.

Lower alkyl groups represented by R³ and R⁴ refers to a same group as the lower alkyl defined above, of which examples specifically include methyl, ethyl and isopropyl.

C₃₋₇ cycloalkyl, represented by R³ and R⁴ and formed by R³ and R⁴ together with the carbon atom to which they are bound, also refers to a same group as the C₃₋₇ cycloalkyl defined above.

It is preferred that both R³ and R⁴ be methyl or that R³ and R⁴ be cyclopropyl formed by R³ and R⁴ together with the carbon atom to which they are bound.

R⁵ is a group selected from the group consisting of:

(1) phenyl, which may be substituted with 1 to 3 same or different groups selected from the group consisting of halogen atoms and lower alkoxy which may be substituted with 1 to 3 same or different halogen atoms;
(2) heteroaryl selected from the group consisting of pyridinyl, pyrazinyl, imidazolyl, thiazolyl, thienyl and oxazolyl, which heteroaryl may be substituted with 1 to 3 same or different groups selected from the group consisting of halogen atoms and lower alkyl which may be substituted with 1 to 3 same or different halogen atoms;
(3) —O—C₃₋₆ branched lower alkyl, which may be substituted with 1 to 3 same or different halogen atoms;
(4) —C₃₋₇ cycloalkyl, which may be substituted with trifluoromethyl; and
(5) —N(H)—C₃₋₆ branched lower alkyl or —N(R⁷)R⁸, wherein N, R⁷ and R⁸ together form a 5-7 membered ring.

Examples of (1) phenyl, represented by R⁵, which phenyl may be substituted with 1 to 3 same or different groups selected from the group consisting of halogen atoms and lower alkoxy which may be substituted with 1 to 3 same or different halogen atoms, include groups represented by phenyl, 4-chlorophenyl, 4-fluorophenyl, 3,4-difluorophenyl, 4-difluoromethoxyphenyl, 4-trifluoromethoxyphenyl, 2-chloro-4-fluorophenyl, etc.

Examples of (2) heteroaryl, represented by R⁵, selected from the group consisting of pyridinyl, pyrazinyl, imidazolyl, thiazolyl, thienyl and oxazolyl, which heteroaryl may be substituted with 1 to 3 same or different groups selected from the group consisting of halogen atoms and lower alkyl which may be substituted with 1 to 3 same or different halogen atoms, include 2-pyridinyl, 6-chloro-2-pyridinyl, 3-fluoro-2-pyridinyl, 5-fluoro-2-pyridinyl, 2-fluoro-4-pyridinyl, 6-fluoro-2-pyridinyl, 5,6-difluoro-2-pyridinyl, 5-methyl-2-pyrazinyl, 1-methyl-2-imidazolyl, 2-thiazolyl, 3-chloro-2-thiazolyl, 3-chloro-2-thienyl and 2-oxazolyl.

(3) —O—C₃₋₆ branched lower alkyl, represented by R⁵, which may be substituted with 1 to 3 same or different halogen atoms, also refers to a group, in which C₃₋₆ branched alkyl of the lower alkyl defined above and an oxygen atom are bound, and specifically encompasses, e.g., isopropoxy, isobutoxy, sec-butoxy, tert-butoxy, etc.

(4) —C₃₋₇ cycloalkyl, represented by R⁵, which may be substituted with trifluoromethyl, also refers to the above-defined unsubstituted C₃₋₇ cycloalkyl or C₃₋₇ cycloalkyl substituted with trifluoromethyl, and specifically encompasses, e.g., 1-(trifluoromethyl)cyclopropyl, 2-(trifluoromethyl)cyclopropyl, 2-(trifluoromethyl)cyclobutyl, 3-(trifluoromethyl)cyclobutyl, 2-(trifluoromethyl)cyclopentyl, 3-(trifluoromethyl)cyclopentyl, 2-(trifluoromethyl)cyclohexyl, 3-(trifluoromethyl)cyclohexyl, 4-(trifluoromethyl)cyclohexyl, 2-(trifluoromethyl)cycloheptyl, 3-(trifluoromethyl)cycloheptyl, 4-(trifluoromethyl)cycloheptyl, etc.

(5) —N(H)—C₃₋₆ branched lower alkyl or —N(R⁷)R⁸ (N, R⁷ and R⁸ together form a 5-7 membered ring), represented by R⁵, refers to a group, in which C₃₋₆ branched alkyl of the lower alkyl defined above and NH are bound, or to a 5-7 membered aliphatic ring containing a nitrogen atom in the ring.

Examples of the —N(H)—C₃₋₆ branched lower alkyl specifically include isopropylamino, isobutylamino, sec-butylamino and tert-butylamino.

Examples of the —N(R⁷)R⁸, wherein N, R⁷ and R⁸ together form a 5-7 membered ring, specifically include 1-pyrrolidinyl, 1-piperidinyl and 1-homopiperidinyl.

R⁶ each independently represents a group selected from the group consisting of a hydrogen atom, lower alkyl, lower alkoxy, a halogen atom, cyano and lower alkoxycarbonylmethyl.

Lower alkyl represented by R⁶ encompasses same groups as the lower alkyl defined above.

Lower alkoxy represented by R⁶ encompasses same groups as the lower alkoxy defined above.

Halogen atoms represented by R⁶ encompass same groups as the halogen atom defined above.

Examples of lower alkoxycarbonylmethyl represented by R⁶ include ethoxycarbonylmethyl, propoxycarbonylmethyl and methoxycarbonylmethyl.

In the formula, m is an integer from 0 to 2, preferably 0 or 1.

In the formula, p is an integer from 1 to 4.

In the formula, q is an integer from 1 to 5.

An aspect of a preferred embodiment of the present invention is a compound or a pharmaceutically acceptable salt thereof in the formula (I), wherein R² is methyl.

Another aspect of a preferred embodiment of the present invention is a compound or a pharmaceutically acceptable salt thereof in the formula (I),

wherein R² is methyl;
both R³ and R⁴ are methyl or cyclopropyl formed by R³ and R⁴ together with the carbon atom to which they are bound; and
m is 0 or 1.

Another aspect of a preferred embodiment of the present invention is a compound or a pharmaceutically acceptable salt thereof in the formula (I),

wherein R² is methyl;
both R³ and R⁴ are methyl or cyclopropyl formed by R³ and R⁴ together with the carbon atom to which they are bound;
m is 0 or 1; and
R⁵ is a group selected from the group consisting of:

phenyl, which may be substituted with 1 or 2 same or different groups selected from the group consisting of fluorine and chlorine atoms, and difluoromethoxy and trifluoromethoxy;

pyridinyl, pyrazinyl, imidazolyl, thiazolyl, thienyl or oxazolyl (the pyridinyl, pyrazinyl, imidazolyl, thiazolyl, thienyl and oxazolyl may be substituted with 1 or 2 same or different groups selected from the group consisting of fluorine and chlorine atoms and methyl;

tert-butoxy, 1-ethylpropoxy or 1-(trifluoromethyl)cyclopropyl;

4-trifluoromethylcyclohexyl; and

tert-butylamino or piperidinyl.

Another aspect of a preferred embodiment of the present invention is a compound or a pharmaceutically acceptable salt thereof in the formula (I),

wherein R² is methyl;
both R³ and R⁴ are methyl or cyclopropyl formed by R³ and R⁴ together with the carbon atom to which they are bound;
m is 0 or 1; and
R⁵ is a group selected from the group consisting of phenyl, 4-chlorophenyl, 4-fluorophenyl, 3,4-difluorophenyl, 4-difluoromethoxyphenyl, 4-trifluoromethoxyphenyl, 5-fluoro-2-pyridinyl, 2-fluoro-4-pyridinyl, 3-chloro-2-thiazolyl, tert-butoxy, 1-(trifluoromethyl)cyclopropyl, 4-trifluoromethylcyclohexyl and tert-butylamino.

Another aspect of a preferred embodiment of the present invention is a compound or a pharmaceutically acceptable salt thereof in the formula (I),

wherein R² is methyl;
both R³ and R⁴ are methyl or cyclopropyl formed by R³ and R⁴ together with the carbon atom to which they are bound;
m is 0 or 1; and
a group represented by the formula (II):

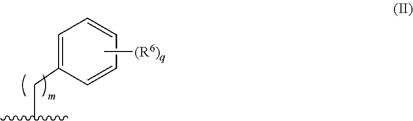

wherein

[symbol]

represents a binding site, in the formula (I) is a group selected from the group consisting of 4-trifluoromethoxyphenyl, 3,5-dichlorophenyl, 2-chlorophenyl, 4-fluoro-3-trifluoromethylphenyl, 2-trifluoromethylphenyl, 2-fluorophenyl, 2-trifluoromethoxyphenyl, 4-trifluoromethoxyphenyl, 3,5-difluorophenyl, 3,4-dichlorophenyl, 3,5-dichlorophenyl, 4-chlorophenyl, 4-chloro-3-fluorophenyl, 3,4-difluorophenyl, 3-methylphenyl, 3-trifluoromethoxyphenyl, 3,5-bis(trifluoromethyl)phenyl and 3-chloro-5-fluorophenyl.

In addition, another aspect of a preferred embodiment of the present invention is a compound or a pharmaceutically acceptable salt thereof in the formula (I),

wherein R² is methyl;
both R³ and R⁴ are methyl or cyclopropyl formed by R³ and R⁴ together with the carbon atom to which they are bound;
m is 0 or 1; and
R⁵ is a group selected from the group consisting of phenyl, 4-chlorophenyl, 4-fluorophenyl, 3,4-difluorophenyl, 4-difluoromethoxyphenyl, 4-trifluoromethoxyphenyl, 5-fluoro-2-pyridinyl, 2-fluoro-4-pyridinyl, 3-chloro-2-thiazolyl, tert-butoxy, 1-(trifluoromethyl)cyclopropyl, 4-trifluoromethylcyclohexyl and tert-butylamino; and a group represented by the formula (II):

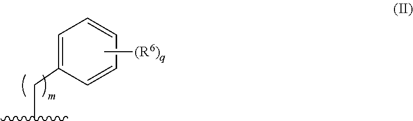

wherein

[symbol]

represents a binding site, in the formula (I) is a group selected from the group consisting of 4-trifluoromethoxyphenyl, 3,5-dichlorophenyl, 2-chlorophenyl, 4-fluoro-3-trifluoromethylphenyl, 2-trifluoromethylphenyl, 2-fluorophenyl, 2-trifluoromethoxyphenyl, 4-trifluoromethoxyphenyl, 3,5-difluorophenyl, 3,4-dichlorophenyl, 3,5-dichlorophenyl, 4-chlorophenyl, 4-chloro-3-fluorophenyl, 3,4-difluorophenyl, 3-methylphenyl, 3-trifluoromethoxyphenyl, 3,5-bis(trifluoromethyl)phenyl and 3-chloro-5-fluorophenyl.

In accordance with a preferred embodiment, any aspects of R¹, R², R³, R⁴, R⁵, R⁶, p, q and m as described above may be combined.

Compounds according to an embodiment of the present invention include, e.g., compound as described in Examples, especially preferably
4-fluoro-N-[2-({(3R)-8-fluoro-1-methyl-2-oxo-5-[4-(tri fluoromethoxy)phenyl]-2,3,4,5-tetrahydro-1H-1,5-benzodiazepin-3-yl}amino)-1,1-dimethyl-2-oxoethyl]benzamide,
N-(2-{[(3R)-5-(3,5-difluorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,4-difluorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,4-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
4-fluoro-N-(2-{[(3R)-8-fluoro-1-methyl-5-(3-methylphenyl)-2-oxo-2,3,4,5-tetrahydro-1H-1,5-benzodiazepin-3-yl]amino}-1,1-dimethyl-2-oxoethyl)benzamide,
N-{1-[({(3R)-8-fluoro-1-methyl-2-oxo-5-[4-(trifluoromethoxy)phenyl]-2,3,4,5-tetrahydro-1H-1,5-benzodiazepin-3-yl}amino)carbonyl]cyclopropyl}benzamide,
N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide,
N-[2-({(3R)-8-fluoro-1-methyl-2-oxo-5-[3-(trifluoromethoxy)phenyl]-2,3,4,5-tetrahydro-1H-1,5-benzodiazepin-3-yl}amino)-1,1-dimethyl-2-oxoethyl]benzamide,
N-[2-({(3R)-5-[3,5-bis(trifluoromethyl)phenyl]-8-fluoro-1-methyl-2-oxo-2,3,4,5-tetrahydro-1H-1,5-benzodiazepin-3-yl}amino)-1,1-dimethyl-2-oxoethyl]-5-fluoropyridin-2-carboxyamide,
N-(2-{[(3R)-5-(3,5-difluorophenyl)-8-fluoro-1-methyl-2-oxo-2,3,4,5-tetrahydro-1H-1,5-benzodiazepin-3-yl]amino}-1,1-dimethyl-2-oxoethyl-5-fluoropyridin-2-carboxyamide,
N-(2-{[(3R)-5-(3-chloro-5-fluorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-2-fluoro isonicotinamide,
N-(2-{[(3R)-8-chloro-5-(3,5-dichlorophenyl)-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-8-chloro-5-(3,5-difluorophenyl)-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-8-chloro-5-(3,5-dichlorophenyl)-1-methyl-2-oxo-2,3,4,5-tetrahydro-1H-1,5-benzodiazepin-3-yl]amino}-1,1-dimethyl-2-oxoethyl)-2-fluoroisonicotinamide,
N-{(3R)-5-[3,5-bis(trifluoromethyl)phenyl]-8-fluoro-1-methyl-2-oxo-2,3,4,5-tetrahydro-1H-1,5-benzodiazepin-3-yl}-2-methyl-N²-{[1-(trifluoromethyl)cyclopropyl]carbonyl}alaninamide,
N-[(3R)-8-chloro-5-(3,5-dichlorophenyl)-1-methyl-2-oxo-2,3,4,5-tetrahydro-1H-1,5-benzodiazepin-3-yl]-2-methyl-N²-{[1-(trifluoromethyl)cyclopropyl]carbonyl}alaninamide,
N-[(3R)-8-chloro-5-(3,5-difluorophenyl)-1-methyl-2-oxo-2,3,4,5-tetrahydro-1H-1,5-benzodiazepin-3-yl]-2-methyl-N²-{[1-(trifluoromethyl)cyclopropyl]carbonyl}alaninamide,
N-[2-({(3S))-5-[3,5-bis(trifluoromethyl)phenyl]-1-methyl-2-oxo-2,3,4,5-tetrahydro-1H-1,5-benzodiazepin-3-yl}amino)-1,1-dimethyl-2-oxoethyl]-4-fluorobenzamide,
N-[1,1-dimethyl-2-({(3R)-1-methyl-2-oxo-5-[4-(trifluoromethoxy)phenyl]-2,3,4,5-tetrahydro-1H-1,5-benzodiazepin-3-yl}amino)-2-oxoethyl]-4-fluorobenzamide,
N-(2-{[(3R)-5-(3,5-dichlorophenyl)-1-methyl-2-oxo-2,3,4,5-tetrahydro-1H-1,5-benzodiazepin-3-yl]amino}-1,1-dimethyl-2-oxoethyl)-4-fluorobenzamide,
N-{1-[({(3R)-8-chloro-1-methyl-2-oxo-5-[4-(trifluoromethoxy)phenyl]-2,3,4,5-tetrahydro-1H-1,5-benzodiazepin-3-yl}amino)carbonyl]cyclopropyl}benzamide,
N-(2-{[(3R)-8-chloro-5-(3,5-difluorophenyl)-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-8-chloro-5-(3,4-dichlorophenyl)-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-1,3-thiazol-2-carboxyamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-(difluoromethoxy)benzamide,
N²-[(tert-butylamino)carbonyl]-N-[(3R)-5-(3,5-dichlorophenyl)-8-fluoro-1-methyl-2-oxo-2,3,4,5-tetrahydro-1H-1,5-benzodiazepin-3-yl]-2-methylalanineamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-(trifluoromethoxy)benzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-3,4-difluorobenzamide,
4-(difluoromethoxy)-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide,
3-chloro-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]thiophene-2-carboxyamide,
N-(2-{[(3R)-5-(3,5-dichlorophenyl)-7-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-7,8-difluoro-1-methyl-2-oxo-2,3,4,5-tetrahydro-1H-1,5-benzodiazepin-3-yl]amino}-1,1-dimethyl-2-oxoethyl)-4-fluorobenzamide,
N-{1-[({(3R)-5-[3,5-bis(trifluoromethyl)benzyl]-1-methyl-2-oxo-2,3,4,5-tetrahydro-1H-1,5-benzodiazepin-3-yl}amino)carbonyl]cyclopropyl}benzamide,
N-[1-[({(3R)-5-(3,5-dichlorobenzyl)-1-methyl-2-oxo-2,3,4,5-tetrahydro-1H-1,5-benzodiazepin-3-yl]amino}carbonyl)cyclopropyl]benzamide,
N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide,
N-[1-({[(3R)-5-(4-chlorobenzyl)-8-fluoro-1-methyl-2-oxo-2,3,4,5-tetrahydro-1H-1,5-benzodiazepin-3-yl]amino}carbonyl)cyclopropyl]benzamide,
N-(2-{[(3R)-5-(3,5-dichlorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
4-fluoro-N-[2-({(3R)-8-fluoro-5-[4-fluoro-3-(trifluoromethyl)benzyl]-1-methyl-2-oxo-2,3,4,5-tetrahydro-1H-1,5-benzodiazepin-3-yl}amino)-1,1-dimethyl-2-oxoethyl]benzamide,
N-(2-{[(3R)-5-(2-chlorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
4-fluoro-N-[2-({(3R)-8-fluoro-1-methyl-2-oxo-5-[2-(trifluoromethyl)benzyl]-2,3,4,5-tetrahydro-1H-1,5-benzodiazepin-3-yl}amino)-1,1-dimethyl-2-oxoethyl]benzamide,
4-fluoro-N-(2-{[(3R)-8-fluoro-5-(2-fluorobenzyl)-1-methyl-2-oxo-2,3,4,5-tetrahydro-1H-1,5-benzodiazepin-3-yl]amino}-1,1-dimethyl-2-oxoethyl)benzamide,
N-[2-({(3R)-5-[2-(difluoromethoxy)benzyl]-8-fluoro-1-methyl-2-oxo-2,3,4,5-tetrahydro-1H-1,5-benzodiazepin-3-yl}amino)-1,1-dimethyl-2-oxoethyl]-4-fluorobenzamide,
N-(2-{[(3R)-5-(4-chloro-3-fluorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
tert-butyl {1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}carbamate,
4-chloro-N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide,
N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}pyridin-2-carboxyamide,
1-ethylpropyl {1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}carbamate,
N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]-4-(trifluoromethyl)cyclohexanecarboxyamide and
N-{1-[({(3R)-7-chloro-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide, more preferably
4-fluoro-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-{1-[({(3R)-8-fluoro-1-methyl-2-oxo-5-[4-(trifluoromethoxy)phenyl]-2,3,4,5-tetrahydro-1H-1,5-benzodiazepin-3-yl}amino)carbonyl]cyclopropyl}benzamide,
N-[(3R)-8-chloro-5-(3,5-dichlorophenyl)-1-methyl-2-oxo-2,3,4,5-tetrahydro-1H-1,5-benzodiazepin-3-yl]-2-methyl-N²-{[1-(trifluoromethyl)cyclopropyl]carbonyl}alaninamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-(difluoromethoxy)benzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-(trifluoromethoxy)benzamide,
4-(difluoromethoxy)-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide,
N-(2-{[(3R)-5-(3,5-dichlorophenyl)-7-fluoro-1-methyl-2-oxo-2,3,4,5-tetrahydro-1H-1,5-benzodiazepin-3-yl]amino}-1,1-dimethyl-2-oxoethyl)-4-fluorobenzamide,
4-fluoro-N-[2-({(3R)-8-fluoro-5-[4-fluoro-3-(trifluoromethyl)benzyl]-1-methyl-2-oxo-2,3,4,5-tetrahydro-1H-1,5-benzodiazepin-3-yl}amino)-1,1-dimethyl-2-oxoethyl]benzamide,
N-(2-{[(3R)-5-(2-chlorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
4-fluoro-N-[2-({(3R)-8-fluoro-1-methyl-2-oxo-5-[2-(trifluoromethyl)-benzyl]-2,3,4,5-tetrahydro-1H-1,5-benzodiazepin-3-yl}amino)-1,1-dimethyl-2-oxoethyl]benzamide,
4-fluoro-N-[2-{(3R)-8-fluoro-5-(2-fluorobenzyl)-1-methyl-2-oxo-2,3,4,5-tetrahydro-1H-1,5-benzodiazepin-3-yl]amino}-1,1-dimethyl-2-oxoethyl)benzamide,
N-[2-({(3R)-5-[2-(difluoromethoxy)benzyl]-8-fluoro-1-methyl-2-oxo-2,3,4,5-tetrahydro-1H-1,5-benzodiazepin-3-yl}amino)-1,1-dimethyl-2-oxoethyl]-4-fluorobenzamide and
tert-butyl {1-[({(3R)-1-methyl-2-oxo-5-[4-trifluoromethoxy)benzyl]-2,3,4,5-tetrahydro-1H-1,5-benzodiazepin-3-yl}amino)carbonyl]cyclopropyl}carbamate.

A process of producing a compound according to an embodiment of the present invention will now be described.

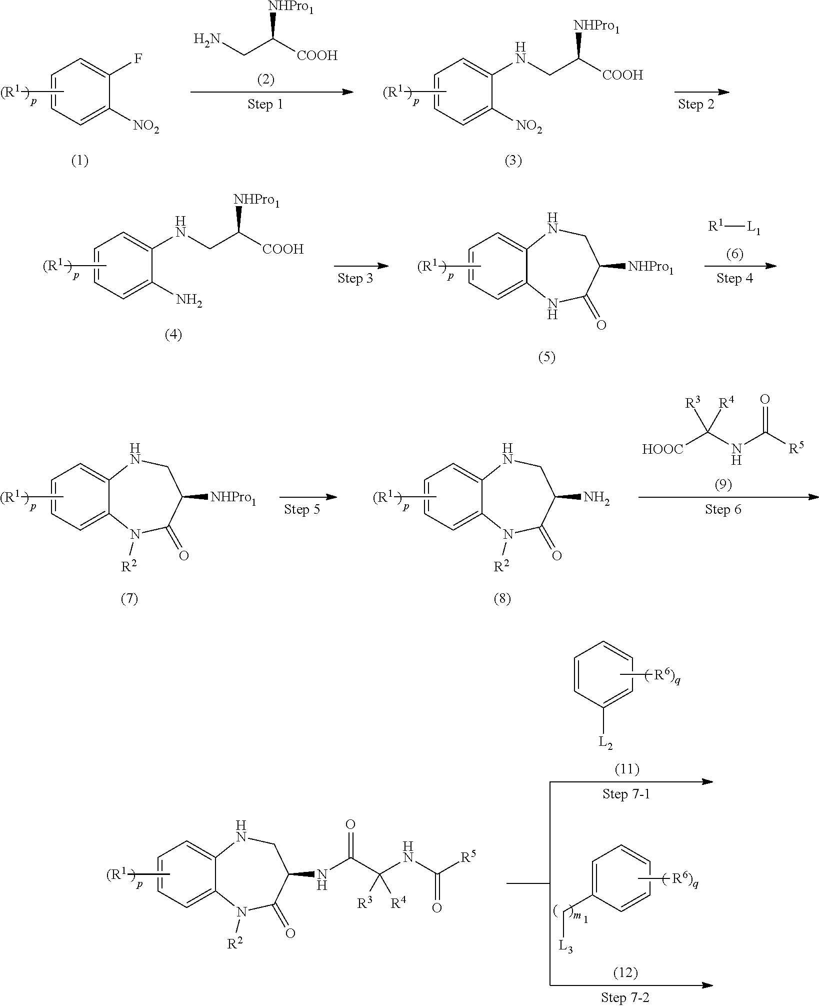

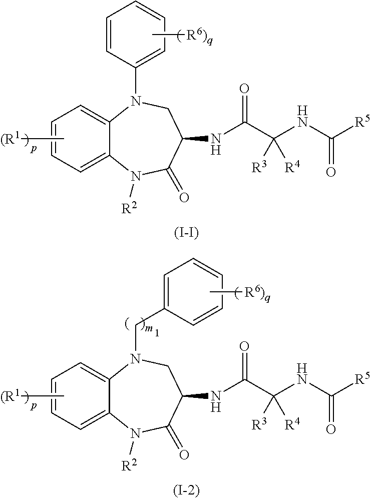

wherein Pro₁ represents a protective group of amino; L₁, L₂ and L₃ each represent a leaving group; m₁ is an integer of 1 or 2; and the other symbols have the same definitions specified above.

### Step 1

This step is a process of producing a compound (3) by reacting a compound (1) with a compound (2) in the presence of base.

Examples of bases as used in this step include potassium carbonate, cesium carbonate, sodium carbonate, triethylamine and diisopropylethylamine.

An amount of the base is typically 1-5 equivalents, preferably 2-3 equivalents, per equivalent of the compound (1).

An amount of the compound (2) used is typically 1-3 equivalents, preferably 1-2 equivalents, per equivalent of the compound (1).

Pro₁ refers to a protective group for amino and encompasses groups as described in documents (e.g., T. W. Green: Protective Groups in Organic Synthesis, Second Edition, John Wiley & Sons (1991), etc.), specifically, e.g., Boc group.

The reaction temperature is typically from room temperature to 80° C., preferably 50-80° C.

The reaction time is typically 6-24 hours, preferably 12-24 hours.

Unless interfering with the reaction, solvents that can be used, include, but are not limited to, e.g., methanol, ethanol, N,N-dimethylformamide, ethyl acetate, tetrahydrofuran, etc., and mixed solvents thereof.

The compound (3) obtained in such a manner may be isolated and purified by well-known separation and purification measures such as concentration, vacuum concentration, reprecipitation, solvent extraction, crystallization and chromatography, or the isolation and purification may be omitted to subject the compound (3) to the subsequent step.

### Step 2

This step is a process for producing a compound (4) by reducing the nitro group of the compound (3).

Methods known to those skilled in the art may be used for reductive reaction in this step. Examples of the reduction methods specifically include: catalytic reduction methods using hydrogen, formic acid, ammonium formate, hydrazine hydrate, etc. and palladium, platinum, nickel catalyst, etc.; reduction methods using ammonium chloride and iron; and reduction methods using methanol and stannous chloride.

An amount of a reducing agent used in this step is typically 1-50 equivalents, preferably 2-20 equivalents, per equivalent of the compound (3).

In addition, in case of reduction by hydrogenation, an amount of the reducing agent used is typically 0.01-0.2 equivalent, preferably 0.05-0.2 equivalent, per equivalent of the compound (3).

The reaction temperature is typically from room temperature to 50° C., preferably from room temperature to 30° C.

The reaction time is typically 1-12 hours, preferably 1-8 hours.

Unless interfering with the reaction, solvents that can be used, include, but are not limited to e.g., methanol, ethanol, N,N-dimethylformamide, ethyl acetate, tetrahydrofuran, etc., and mixed solvents thereof.

The compound (4) obtained in such a manner may be isolated and purified by well-known separation and purification measures such as concentration, vacuum concentration, reprecipitation, solvent extraction, crystallization and chromatography, or the isolation and purification may be omitted to subject the compound (4) to the subsequent step.

### Step 3

This step is a process for producing a compound (5) by condensing the compound (4) in a molecule in the presence of base and a condensation agent.

Bases as used include, e.g., dimethylaminopyridine, triethylamine, pyridine and diisopropylethylamine.

An amount of the base is typically 1-2 equivalents, preferably 1-1.5 equivalents, per equivalent of the compound (4).

A condensation adjuvant may be also added into a reaction system.

Condensation agents as used include, e.g., carbonyldiimidazole, N,N-dicyclohexylcarbodiimide, 1-ethyl-3-(3-dimethylaminopropyl) carbodiimide, diphenylphosphoryl azide and dipyridyl disulfide-triphenylphosphine.

An amount of the condensation agent is typically 1-2 equivalents, preferably 1-1.5 equivalents, per equivalent of the compound (4).

Condensation adjuvants include, e.g., N-hydroxybenzotriazole hydrate and N-hydroxysuccinimide.

An amount of a condensation adjuvant used is typically 1-2 equivalents, preferably 1-1.5 equivalents, per equivalent of the compound (4).

The reaction temperature is typically from room temperature to 80° C., preferably from room temperature to 50° C.

The reaction time is typically 1-24 hours, preferably 1-12 hours.

Unless interfering with the reaction, reaction solvents that can be used, include, but are not limited to e.g., DMF, dichloromethane, chloroform, THF, etc., and mixed solvents thereof.

The compound (1) obtained in such a manner may be isolated and purified by well-known separation and purification measures such as concentration, vacuum concentration, reprecipitation, solvent extraction, crystallization and chromatography, or the isolation and purification may be omitted to subject the compound (1) to the subsequent step.

### Step 4

This step is a process for producing a compound (7) by reacting the compound (5) with the compound (6) in the presence of base.

Bases as used include, e.g., sodium tert-amyloxide, sodium tert-butoxide and sodium hydride.

An amount of the base is typically 1-5 equivalents, preferably 1-2 equivalents, per equivalent of the compound (5).

Compounds (6) used specifically include, e.g., methyl iodide, ethyl iodide, isopropyl iodide and fluoromethyl tosylate.

An amount of the compound (6) is typically 1-5 equivalents, preferably 1-2 equivalents, per equivalent of the compound (5).

The reaction temperature is typically from −20° C. to room temperature, preferably from −20 to 0° C.

The reaction time is typically 1-8 hours, preferably 1-3 hours.

Unless interfering with the reaction, reaction solvents that can be used, include, but are not limited to e.g., DMF, NMP, DMA, THF, ether, etc., and mixed solvents thereof.

The compound (7) obtained in such a manner may be isolated and purified by well-known separation and purification measures such as concentration, vacuum concentration, reprecipitation, solvent extraction, crystallization and chromatography, or the isolation and purification may be omitted to subject the compound (7) to the subsequent step.

### Step 5

This step is a process for producing a compound (8) by removing a protective group Pro₁ of the amino group that the compound (7).

The reaction in this step can be carried out by methods as described in documents (e.g., T. W. Green: Protective Groups in Organic Synthesis, Second Edition, John Wiley & Sons (1991), etc.), other methods known in the art and combinations thereof.

The compound (8) obtained in such a manner may be isolated and purified by well-known separation and purification measures such as concentration, vacuum concentration, reprecipitation, solvent extraction, crystallization and chromatography, or the isolation and purification may be omitted to subject the compound (8) to the subsequent step.

### Step 6

This step is a process for producing a compound (10) by reacting the compound (8) with a compound (9) or a reactive derivative thereof.

For this reaction, typical amide formation reaction may be performed by methods as described in documents (e.g., Nobuo Izumiya, et al.: Peptide Gosei no Kiso to Jikken (Fundamentals and Experiments of Peptide Synthesis), Maruzen (1983); Comprehensive Organic Synthesis, Vol. 6, Pergamon Press (1991), etc.), other methods known in the art and combinations thereof, that is, by using a condensation agent that is well known to those skilled in the art, or by an ester activation method, a mixed anhydride method, an acid chloride method, a carbodiimide method, etc., which can be used by those skilled in the art. Examples of such amide formation reagents include thionyl chloride, oxalyl chloride, N,N-dicyclohexylcarbodiimide, 1-methyl-2-bromopyridinium iodide, N,N′-carbonyldiimidazole, diphenylphosphoryl chloride, diphenylphosphoryl azide, N,N′-disuccinimidyl carbonate, N,N′-disuccinimidyl oxalate, 1-ethyl-3-(3-dimethylaminopropyl)carbodiimide hydrochloride, ethyl chloroformate, isobutyl chloroformate and benzotriazol-1-yl-oxy-tris(dimethylamino)phosphonium hexafluorophosphate; especially preferably, e.g., thionyl chloride, 1-ethyl-3-(3-dimethylaminopropyl)carbodiimide hydrochloride, N,N-dicyclohexylcarbodiimide and benzotriazol-1-yl-oxy-tris(dimethylamino)phosphonium hexafluorophosphate. For the amide formation reaction, base and a condensation adjuvant may be also used together with the amide formation reagent.

Bases as used include ternary aliphatic amines such as trimethylamine, triethylamine, N,N-diisopropylethylamine, N-methylmorpholine, N-methylpyrrolidine, N-methylpiperidine, N,N-dimethylaniline, 1,8-diazabicyclo[5.4.0]undeca-7-en (DBU) and 1,5-azabicyclo[4.3.0]nona-5-en (DBN); and aromatic amines such as pyridine, 4-dimethylaminopyridine, picoline, lutidine, quinoline and isoquinoline; especially preferably, e.g., ternary aliphatic amines, etc., particularly preferably, e.g., trimethylamine, N,N-diisopropylethylamine, etc.

An amount of the base is typically 1-10 equivalents, preferably 1-5 equivalents, per equivalent of the compound (9) or a reactive derivative thereof.

Condensation adjuvants as used include, for example, N-hydroxybenzotriazole hydrate, N-hydroxy succinimide, N-hydroxy-5-norbornen-2,3-dicarboximide and 3-hydroxy-3,4-dihydro-4-oxo-1,2,3-benzotriazole; especially preferably, e.g., N-hydroxybenzotriazole, etc.

An amount of the condensation adjuvant is typically 1-10 equivalents, preferably 1-2 equivalents, per equivalent of the compound (9) or a reactive derivative thereof.

An amount of the compound (8) used is typically 1-10 equivalents, preferably 1-2 equivalents per equivalent of the compound (9) or a reactive derivative thereof.

Unless interfering with the reaction, reaction solvents that can be used, include, but are not limited to e.g., inactive solvents; specifically, e.g., DMF, methylene chloride, chloroform, 1,2-dichloroethane, dimethylformamide, ethyl acetate, methyl acetate, acetonitrile, benzene, xylene, toluene, 1,4-dioxane, tetrahydrofuran and dimethoxyethane or mixed solvents thereof; preferably, e.g., methylene chloride, chloroform, 1,2-dichloroethane, acetonitrile and N,N-dimethylformamide, from the viewpoint of ensuring preferable reaction temperature.

The reaction time is typically 1-24 hours, preferably 1-12 hours.

The reaction temperature is typically from 0° C. to the boiling point of a solvent, preferably from room temperature to 80° C.

One or a combination of two or more of bases, amide formation reagents and condensation adjuvants as used in this step may be used.

The compound (10) obtained in such a manner may be isolated and purified by well-known separation and purification measures such as concentration, vacuum concentration, crystallization, solvent extraction, reprecipitation and chromatography.

### Step 7-1

This step is a process for producing a compound (1-1) according to an embodiment of the present invention by reacting the compound (10) with a compound (11) in the presence of base, palladium catalyst and phosphine ligand.

Bases as used include, e.g., potassium carbonate, cesium carbonate, sodium carbonate, sodium tert-amyloxide, sodium tert-butoxide, lithium hexamethyldisilazide, triethylamine and diisopropylamine.

An amount of the base is typically 1-10 equivalents, preferably 1-5 equivalents, per equivalent of the compound (10).

Palladium catalysts as used include, e.g., Pd₂(dba)₃, palladium acetate, palladium chloride and allylpalladium chloride dimer.

An amount of palladium catalyst as used is typically 0.01-0.2 equivalent, preferably 0.05-0.2 equivalent, per equivalent of the compound (10).

Phosphine ligands include, e.g., 2-dicyclohexylphosphino-2′,4′,6′-triisopropylbiphenyl (also referred to as “XPhos”).

An amount of the phosphine ligand is typically 0.02-0.4 equivalent, preferably 0.1-0.4 equivalent, per equivalent of the compound (10).

Unless interfering with the reaction, reaction solvents as used in this step, which are not particularly limited, specifically include, e.g., tert-butanol, toluene, 1,4-dioxan and tetrahydrofuran.

The reaction time is typically 1-48 hours, preferably 1-12 hours.

The reaction temperature is typically from room temperature to the boiling point of a solvent, preferably from 90° C. to the boiling point of the solvent.

The compound (1-1) obtained in such a manner may be isolated and purified by well-known separation and purification measures such as concentration, vacuum concentration, reprecipitation, solvent extraction, crystallization and chromatography.

### Step 7-2

This step is a process for producing a compound (1-2) according to an embodiment of the present invention by reacting the compound (10) with a compound (12) in the presence of base.

Bases as used include, e.g., potassium carbonate, sodium carbonate, cesium carbonate and sodium tert-butoxide.

An amount of the base is typically 1-10 equivalents, preferably 1-3 equivalents, per equivalent of the compound (10).

An amount of the compound (12) is typically 1-10 equivalents, preferably 1-2 equivalents, per equivalent of the compound (10).

Unless interfering with the reaction, reaction solvents as used, which are not particularly limited, specifically include, e.g., DMF, NMP, DMA and DMSO.

In this step, NaI or KI may be also added to promote the reaction.

An amount of NaI or KI as used is typically 1-10 equivalents, preferably 1-5 equivalents, per equivalent of the compound (10).

The reaction time is typically up to 12 hours, preferably 1-6 hours.

The reaction temperature is typically from room temperature to 80° C., preferably from 50° C. to 80° C.

The compound (1-2) obtained in such a manner may be isolated and purified by well-known separation and purification measures such as concentration, vacuum concentration, reprecipitation, solvent extraction, crystallization and chromatography.

The benzodiazepine-2-on derivative in accordance with an embodiment of the present invention may be present as a pharmaceutically acceptable salt, which may be produced according to usual methods using the compound (I), (I-1) or (I-2).

Examples of such acid addition salts include hydrohalic acid salts such as hydrochloride, hydrofluorate, hydrobromide and hydroiodide; inorganic acid salts such as nitride, perchlorate, sulfate, phosphate and carbonate; lower alkyl sulfonate salts such as methanesulfonate, trifluoromethanesulfonate and ethanesulfonate; aryl sulfonates such as benzensuplhonate and p-toluenesulfonate; organic salts such as fumarate, succinate, citrate, tartrate, oxalate and maleate; and acid addition salts of organic acids, e.g., amino acids, such as glutamate and aspartate.

When the compound according to an embodiment of the present invention has an acidic group, such as carboxyl, in the group, the compound can be also converted into a corresponding pharmaceutically acceptable salt by processing the compound with a base. Examples of such base addition salts include alkali metal salts such as sodium and potassium; alkaline earth metal salts such as calcium and magnesium; ammonium salts; and salts of organic bases such as guanidine, triethylamine and dicyclohexylamine.

Furthermore, the compound according to an embodiment of the present invention may be present in the form of a free compound or any hydrate or solvate of a salt thereof.

In contrast, a salt or ester can be also converted into a free compound by a usual method.

Furthermore, in the compound according to an embodiment of the present invention, a stereoisomer or a tautomer, such as an optical isomer, a diastereoisomer or a geometrical isomer, is sometimes present depending on the form of a substituent. It will be appreciated that these isomers are encompassed entirely by compounds according to an embodiment of the present invention. Furthermore, it will be appreciated that any mixture of these isomers is encompassed by compounds according to an embodiment of the present invention.

A compound represented by the general formula (I) may be orally or parenterally administered and is formulated into a form suitable for such administration to provide an agent for treating and/or preventing hyperlipidemia, diabetes and obesity using the compound.

When the compound according to an embodiment of the present invention is clinically used, a pharmaceutically acceptable additive may be also added, depending on a dosage form, to produce various preparations, followed by administration of the preparations. Additives in this case, for which various additives that are usually used in the field of formulation, include, for example, gelatine, lactose, saccharose, titanium oxide, starch, microcrystalline cellulose, hydroxypropyl methylcellulose, carboxymethyl cellulose, corn starch, microcrystalline wax, white petrolatum, magnesium aluminometasilicate, anhydrous calcium phosphate, citric acid, trisodium citrate, hydroxypropylcellulose, sorbitol, sorbitan fatty acid esters, polysorbates, sucrose fatty acid esters, polyoxyethylene, hydrogenated castor oil, polyvinyl pyrrolidone, magnesium stearate, light anhydrous silicic acid, talc, vegetable oil, benzyl alcohol, gum arabic, propylene glycol, polyalkylene glycol, cyclodextrin, hydroxypropyl cyclodextrin, etc.

Examples of dosage forms as formulated mixtures with such additives include solid preparations such as tablets, capsules, granules, powders and suppositories; and liquid preparations such as syrups, elixirs and injectables, which can be prepared according to typical methods in the field of formulation. Further, the liquid preparations may be in the form of dissolution or suspension in water or another appropriate medium just before use. Particularly, the injectables may be also dissolved or suspended in a physiological saline solution or a glucose solution as needed, and a buffer or a preservative may be further added to the mixture.

Such preparations may contain the compound according to an embodiment of the present invention at a rate of 1.0-100%, preferably 1.0-60%, by weight of the total drug. Such preparations may also contain other therapeutically-effective compounds.

The compound according to an embodiment of the present invention may be used in combination with a drug efficacious for hyperlipidemia, diabetes, obesity or the like (hereinafter referred to as “concomitant drug”). Such drugs may be administered concurrently, separately or sequentially in treatment or prevention of the diseases. When the compound according to an embodiment of the present invention is used concurrently with one or more concomitant drugs, they may be formed into a pharmaceutical composition in a single dosage form. In a combination therapy, however, a composition containing the compound according to an embodiment of the present invention and a concomitant drug in different packages may be administered concurrently, separately or sequentially to an administration subject. They may be also administered at intervals.

A dose of a concomitant drug may be based on a dose which is clinically used and may be selected appropriately depending on an administration subject, an administration route, a disease, a combination and the like. A dosage form of such a concomitant drug is not particularly limited, and it may be any form in which the compound according to an embodiment of the present invention and a concomitant drug are combined when they are administered. Examples of such dosage forms include (1) administration of a single pharmaceutical preparation obtained by formulating the compound according to an embodiment of the present invention and a concomitant drug concurrently; (2) coadministration via the same administration route of two pharmaceutical preparations obtained by formulating the compound according to an embodiment of the present invention and a concomitant drug separately; (3) administration at an interval via the same administration route of two pharmaceutical preparations obtained by formulating the compound according to an embodiment of the present invention and a concomitant drug separately; (4) coadministration via different administration routes of two pharmaceutical preparations obtained by formulating the compound according to an embodiment of the present invention and a concomitant drug separately; and (5) administration at an interval via different administration routes of two pharmaceutical preparations obtained by formulating the compound according to an embodiment of the present invention and a concomitant drug separately (e.g. administration of the compound according to an embodiment of the present invention and then a concomitant drug, or administration in the reverse order). The blending ratio of the compound according to an embodiment of the present invention and a concomitant drug may be selected appropriately depending on an administration subject, an administration route, a disease, and the like.

When the compound according to an embodiment of the present invention is used in clinical fields, a dosage regimen of it depends on the sex, age, body weight and severity of condition of a patient; and the type and range of desired therapeutic effect. In case of oral administration to an adult human, the usual dosage regimen of it is 0.01-100 mg/kg per day, preferably 0.03-1 mg/kg per day in one dose or several divided doses. In case of parenteral administration, it is 0.001-10 mg/kg per day, preferably 0.001-0.1 mg/kg per day in one dose or several divided doses.

Any appropriate administration route may be used to administer an effective amount of the compound according to an embodiment of the present invention to a mammal, particularly to a human. For example, oral, rectum, local, intravenous, ocular, lung and nasal administration routes may be used. Examples of dosage forms include tablets, troches, powders, suspensions, solutions, capsules, creams, aerosols, etc., in which tablets for oral use are preferred.

For preparing compositions for oral use, any typical pharmaceutical medium may be used, examples of which include water, glycol, oils, alcohols, flavoring agents, preservatives, coloring agents, etc. For preparing liquid compositions for oral use, examples of pharmaceutical media include suspensions, elixirs and solutions, and examples of carriers include starches, sugars, microcrystalline celluloses, diluents, granulating agents, lubricants, binders and disintegrating agents. For preparing solid compositions for oral use, examples of pharmaceutical media include powders, capsules and tablets. Particularly, the solid compositions for oral use are preferred.

Because of their ease of administration, tablets and capsules represent the most advantageous oral dosage unit form. If desired, tablets can be coated with standard aqueous or non-aqueous techniques.

In addition to the common dosage forms described above, the compounds according to the formula (I) may also be administered by controlled release means and/or delivery devices that are described in U.S. Pat. Nos. 3,845,770; 3,916,899; 3,536,809; 3,598,123; 3,630,200 and 4,008,719.

Pharmaceutical compositions in accordance with an embodiment of the present invention suitable for oral administration include capsules, cachets or tablets, each containing a predetermined amount of an active ingredient, such as a powder or granules, or as an aqueous liquid, a non-aqueous liquid, an oil-in-water emulsion or a water-in-oil liquid emulsion. Such compositions may be prepared by any pharmaceutical method, including a method of combining an active ingredient with a carrier consisting of one or more necessary constituents.

In general, compositions are prepared by uniformly and sufficiently mixing active ingredients with liquid carriers or finely divided solid carriers, or both, and then shaping the product into the desired form if necessary. For example, a tablet can be prepared optionally together with one or more accessory ingredients by compression or molding. Compressed tablets can be prepared by compressing, in a suitable machine, the active ingredients in a free-flowing form such as powder or granules, optionally mixed with a binder, a lubricant, an inert excipient, a surfactant or a dispersive agent.

Molded tablets can be prepared by molding, in a suitable machine, a mixture of the powdered compound moistened with an inert liquid diluent.

Preferably, each tablet contains about 1 mg to 1 g of active ingredient, and each cachet or capsule contains about 1 mg to 500 mg of active ingredient.

Examples of pharmaceutical dosage forms for the compound of the formula (I) are shown below.

TABLE 1

| Suspension for injection | |
|---|---|
| (I. M.) | mg/ml |
| Compound of formula (I) | 10 |
| Methyl cellulose | 5.0 |
| TWEEN 80 | 0.5 |
| Benzyl alcohol | 9.0 |
| Benzalkonium chloride | 1.0 |

Adjusted to 1.0 ml by addition of water for injection.

TABLE 2

| Tablet | mg/tablet |
|---|---|
| Compound of formula (I) | 25 |
| Methyl cellulose | 415 |
| TWEEN 80 | 14.0 |
| Benzyl alcohol | 43.5 |
| Magnesium stearate | 2.5 |
| Total: | 500 ng |

TABLE 3

| Capsule | mg/capsule |
|---|---|
| Compound of formula (I) | 25 |
| Lactose powder | 573.5 |
| Magnesium stearate | 1.5 |
| Total: | 600 ng |

TABLE 4

| Aerosol | Per container |
|---|---|
| Compound of formula (I) | 24 mg |
| Lecithin, NF Liq. Conc. | 1.2 mg |
| Trichlorofluoromethane, | 4.025 g |
| NF | |
| Dichlorodifluoromethane, | 12.15 g |
| NF | |

The compound of the formula (I) may be used in combination with other drugs used in treatment/prevention/delay of onset of hyperlipidemia, diabetes or obesity as well as diseases or conditions associated therewith. The other drugs may be administered in an administration route or a dose that is typically used, concurrently with or separately from the compound of the formula (I).

When the compound of the formula (I) is used concurrently with one or more drugs, a pharmaceutical composition containing the compound of the formula (I) and the other drugs is preferred.

Accordingly, the pharmaceutical composition according to an embodiment of the present invention contains the compound of the formula (I) as well as other active ingredients that are one or more. Examples of active ingredients which are used in combination with the compound of the formula (I) include, but are not limited to, the following (a) to (i):

(a) other DGAT1 inhibitors;
(b) glucokinase activators;
(C) biguanides (e.g., buformin, metformin and phenformin);
(d) PPAR agonists (e.g., troglitazone, pioglitazone and rosiglitazone);
(e) insulin;
(f) somatostatin;
(g) α-glucosidase inhibitors (e.g., voglibose, miglitol and acarbose);
(h) insulin secretagogues (e.g., acetohexamide, carbutamide, chlorpropamide, glybenclamide, gliclazide, glimepiride, glipizide, gliquidone, glisoxepide, glyburide, glyhexamide, glypinamide, phenbutamide, tolazamide, tolbutamide, tolcyclamide, nateglinide and repaglinide); and
(i) DPP-IV (dipeptidyl peptidase-IV inhibitors, e.g., sitagliptin),
which may be administered separately or in the same pharmaceutical composition.

A weight ratio of the compound of the formula (I) to a second active ingredient varies within wide limits and further depends on the effective dose of each active ingredient. Accordingly, for example, when the compound of the formula (I) is used in combination with a PPAR agonist, a weight ratio of the compound of the formula (I) to the PPAR agonist is generally about 1000:1 to 1:1000, preferably about 200:1 to 1:200. Combinations of the compound of the formula (I) and other active ingredients are within the above-mentioned range; and in any case, the effective dose of each active ingredient should be used.

The compound according to an embodiment of the present invention or a pharmaceutically acceptable salt thereof has strong DGAT1 inhibitory activity and is thus useful for treating and/or preventing hyperlipidemia, diabetes and obesity.

It should be understood by those skilled in the art that various modifications, combinations, sub-combinations and alterations may occur depending on design requirements and other factors insofar as they are within the scope of the appended claims or the equivalents thereof.

### EXAMPLES

The present invention is described below in more detail referring to Examples and Reference Examples, but is not limited thereto.

### Formulation Example 1

Ten parts of the compound in accordance with Example 1, 15 parts of heavy magnesium oxide and 75 parts of lactose were blended uniformly to prepare a powder having a particle size of 350 μm or less in powder or granular form. The powder was charged in a capsule container to form a capsule.

### Formulation Example 2

After uniformly blending 45 parts of the compound in accordance with Example 1, 15 parts of starch, 16 parts of lactose, 21 parts of crystalline cellulose, 3 parts of poly vinyl alcohol and 30 parts of distilled water, the blend was crushed into granules, which were dried and then sieved to form granules having a particle diameter of 177-1410 μm.

### Formulation Example 3

After preparing granules in the same manner as in Formulation Example 2, 3 parts of calcium stearate was added to 96 parts of the granules, and the mixture was compression-molded to prepare tablets having a diameter of 10 mm.

### Formulation Example 4

To 90 parts of the granules prepared by the method described in Formulation Example 2 was added 10 parts of crystalline cellulose and 3 parts of calcium stearate, and the mixture was compression-molded to form tablets having a diameter of 8 mm, to which a syrup gelatin/precipitated calcium carbonate suspension was added to prepare sugar-coated tablets.

Wakogel (registered trademark) C-300, made by Wako Pure Chemical Industries Ltd., or KP-Sil (Registered Trademark) Silica prepacked column, made by Biotage, was used for the silica gel column chromatography in Examples. Kieselgel™ 60 F₂₅₄, Art. 5744, made by Merck & Co., was used for preparative thin layer chromatography. Chromatorex (registered trademark) NH (100-250 mesh or 200-350 mesh), made by Fuji Silysia Chemical Ltd., was used for basic silica gel column chromatography.

¹H-NMR was measured using Gemini (200 MHz, 300 MHz), Mercury (400 MHz) and Inova (400 MHz), made by Varian, using tetramethylsilane as a standard substance. In addition, the mass spectra were measured by electrospray ionization (ESI) or atmospheric pressure chemical ionization (APCI) using Micromass ZQ made by Waters.

The meanings of the abbreviations in Examples are shown below.

i-Bu=isobutyl
n-Bu=n-butyl
t-Bu=tert-butyl
Boc=tert-butoxycarbonyl
Me=methyl
Et=ethyl
Ph=phenyl
i-Pr=isopropyl
n-Pr=n-propyl
CDCl₃=heavy chloroform
CD₃OD=heavy methanol
DMSO-d₆=heavy dimethylsulfoxide

The meanings of the abbreviations in the nuclear magnetic resonance spectra are shown below.

s=singlet
d=doublet
dd=double doublet
dt=double triplet
ddd=double double doublet
Sept=septet
t=triplet
m=multiplet
br=broad
brs=broad singlet
q=quartet
J=coupling constant
Hz=hertz

### Example 1

### Synthesis of 4-fluoro-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide

A compound obtained in Reference Example 3 was dissolved in t-BuOH under nitrogen atmosphere, 1-bromo-4-(trifluoromethoxy)benzene, potassium carbonate, 2-dicyclohexyl-2′,4′,6′-triisopropylbiphenyl (X-Phos) and tris(dibenzylideneacetone)dipalladium (Pd₂(dba)₃) were added to the mixture, and the mixture was subjected to a deaeration operation, followed by stirring the reaction liquid overnight at 90° C. The reaction liquid was cooled to room temperature, thereafter poured into a saturated aqueous ammonium chloride solution, and extracted with ethyl acetate twice. The extract was washed with water and a saturated saline solution and dried over anhydrous sodium sulfate. Sodium sulfate was filtered off, and the filtrate was removed under reduced pressure. The residue was purified through silica gel chromatography (hexane:ethyl acetate=100:0-40:60) to yield the compound of interest as a white solid.

The analytical data of the title compound are shown below.

¹H-NMR (CDCl₃) δ: 1.69 (6H, s), 3.39 (3H, s), 3.56 (1H, dd, J=12.0, 8 Hz), 4.24 (1H, dd, J=12.0, 8 Hz), 4.74 (1H, d t, J=12.0, 8 Hz), 6.67 (2H, d, J=8 Hz), 6.76 (1H, s), 6.95-7.22 (8H, m), 7.79 (2H, dd, J=12.0, 8 Hz).

ESI-MS (m/e): 557 [M+H]+

### Example 2

### Synthesis of N-(2-[{(3R)-5-(3,5-difluorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a colorless oily substance by the method as in Example 1, using 1-bromo-3,5-difluorobenzene.

The analytical data of the title compound are shown below.

¹H-NMR (CDCl₃) δ: 1.69 (3H, s), 1.70 (3H, s), 3.39 (3H, s), 3.57 (1H, dd, J=12.0, 8 Hz), 4.18 (1H, dd, J=12.0, 8 Hz), 4.75 (1H, dt, J=12.0, 8 Hz), 6.15 (2H, dd, J=10.0, 2.2 Hz), 6.30 (1H, tt, J=8.8, 2.2 Hz), 6.75 (1H, s), 7.08-7.26 (6H, m), 7.80 (2H, dd, J=8.0, 4 Hz).

ESI-MS (m/e): 529 [M+H]+

### Example 3

### Synthesis of N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a pale yellow solid by the method as in Example 1, using 1-bromo-3,5-dichlorobenzene.

The analytical data of the title compound are shown below.

¹H-NMR (CDCl₃) δ: 1.69 (3H, s), 1.70 (3H, s), 3.40 (3H, s), 3.54 (1H, dd, J=12.0, 8 Hz), 4.20 (1H, dd, J=12.0, 8 Hz), 4.72 (1H, dt, J=12.0, 8 Hz), 6.51 (2H, d, J=1.6 Hz), 6.73 (1H, s), 6.83 (1H, m), 7.00-7.23 (6H, m), 7.78-7.82 (2H, m).

ESI-MS (m/e): 562 [M+H]+

### Example 4

### Synthesis of 4-fluoro-N-(2-{[(3R)-8-fluoro-5-(3-fluorophenyl)-1-methyl-2-oxo-2,3,4,5-tetrahydro-1H-1,5-benzodiazepin-3-yl]amino}-1,1-dimethyl-2-oxoethyl)benzamide

The title compound was obtained as a colorless oily substance by the method as in Example 1, using 1-bromo-3-fluorobenzene.

The analytical data of the title compound are shown below.

¹H-NMR (CDCl₃) δ: 1.73 (3H, s), 1.74 (3H, s), 3.43 (3H, s), 3.61 (1H, dd, J=12.0, 8 Hz), 4.25 (1H, dd, J=12.0, 8 Hz), 4.78 (1H, dt, J=12.0, 8 Hz), 6.38-6.42 (1H, m), 6.47-6.49 (1H, m), 6.59 (1H, dt, J=8.0, 4 Hz), 6.82 (1H, s), 7.03-7.05 (1H, m), 7.08 (1H, dd, J=9.0, 2.7 Hz), 7.13-7.20 (4H, m), 7.27 (1H, dd, J=8.0, 4 Hz), 7.83 (2H, dd, J=12.0, 8 Hz).

ESI-MS (m/e): 511 [M+H]+

### Example 5

### Synthesis of 4-fluoro-N-[2-({(3R)-8-fluoro-1-methyl-2-oxo-5-[3-(trifluoromethoxy)phenyl]-2,3,4,5-tetrahydro-1H-1,5-benzodiazepin-3-yl}amino)-1,1-dimethyl-2-oxoethyl]benzamide

The title compound was obtained as a colorless oily substance by the method as in Example 1, using 1-bromo-3-(trifluoromethoxy)benzene.

The analytical data of the title compound are shown below.

¹H-NMR (CDCl₃) δ: 1.69 (3H, s), 1.70 (3H, s), 3.39 (3H, s), 3.57 (1H, dd, J=11.3, 9.7 Hz), 4.22 (1H, dd, J=9.7, 6.5 Hz), 4.72-4.78 (1H, m), 6.47 (1H, s), 6.59 (1H, dd, J=8.2, 2.3 Hz), 6.71 (1H, d, J=8.2 Hz), 6.79 (1H, s), 6.97-7.25 (7H, m), 7.79 (2H, dd, J=12.0, 8 Hz).

ESI-MS (m/e): 577 [M+H]+

### Example 6

### Synthesis of N-(2-{[(3R)-5-(3-cyanophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 1, using 1-bromo-3-cyanobenzene.

The analytical data of the title compound are shown below.

¹H-NMR (CDCl₃) δ: 1.69 (3H, s), 1.70 (3H, s), 3.40 (3H, s), 3.57 (1H, dd, J=11.2, 9.8 Hz), 4.25 (1H, dd, J=9.8, 6.6 Hz), 4.73-4.79 (1H, m), 6.70 (1H, s), 6.88-6.91 (2H, m), 6.99-7.20 (7H, m), 7.24-7.30 (1H, m), 7.77-7.82 (2H, m).

ESI-MS (m/e): 518 [M+H]+

### Example 7

### Synthesis of 4-fluoro-N-(2-{[(3R)-8-fluoro-5-(2-fluorophenyl)-1-methyl-2-oxo-2,3,4,5-tetrahydro-1H-1,5-benzodiazepin-3-yl]amino}-1,1-dimethyl-2-oxoethyl)benzamide

The title compound was obtained as a white solid by the method as in Example 1, using 1-bromo-2-fluorobenzene.

The analytical data of the title compound are shown below.

1H-NMR (CDCl3) δ: 1.70 (6H, s), 3.46-3.40 (4H, m), 4.42 (1H, dd, J=9.3, 6.8 Hz), 4.73-4.80 (1H, m), 6.82-7.01 (6H, m), 7.06-7.16 (5H, m), 7.77-7.81 (2H, m).

ESI-MS (m/e): 511 [M+H]+

### Example 8

### Synthesis of N-(2-{[(3R)-5-(3,4-difluorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 1, using 1-bromo-3,4-difluorobenzene.

The analytical data of the title compound are shown below.

1H-NMR (CDCl3) δ: 1.69 (3H, s), 1.69 (3H, s), 3.40 (3H, s), 3.50 (1H, dd, J=11.2, 9.3 Hz), 4.21 (1H, dd, J=9.3, 6.6 Hz), 4.68-4.75 (1H, m), 6.36-6.40 (1H, m), 6.45-6.50 (1H, m), 6.73 (1H, s), 6.95-7.05 (3H, m), 7.08-7.18 (4H, m), 7.77-7.81 (2H, m).

ESI-MS (m/e): 529 [M+H]+

### Example 9

### Synthesis of N-(2-{[(3R)-5-(3,4-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a pale yellow solid by the method as in Example 1, using 1-bromo-3,4-dichlorobenzene.

The analytical data of the title compound are shown below.

1H-NMR (CDCl3) δ: 1.69 (3H, s), 1.70 (3H, s), 3.40 (3H, s), 3.53 (1H, dd, J=11.7, 9.8 Hz), 4.21 (1H, dd, J=9.8, 6.6 Hz), 4.69-4.75 (1H, m), 6.51 (1H, dd, J=8.8, 2.9 Hz), 6.75-6.72 (2H, m), 6.97-7.06 (2H, m), 7.09-7.13 (2H, m), 7.16-7.23 (3H, m), 7.77-7.81 (2H, m).

ESI-MS (m/e): 561 [M+H]+

### Example 10

### Synthesis of 4-fluoro-N-(2-{[(3R)-8-fluoro-1-methyl-5-(3-methylphenyl)-2-oxo-2,3,4,5-tetrahydro-1H-1,5-benzodiazepin-3-yl]amino}-1,1-dimethyl-2-oxoethyl)benzamide

The title compound was obtained as a pale yellow solid by the method as in Example 1, using 1-bromo-3-methylbenzene.

The analytical data of the title compound are shown below.

1H-NMR (CDCl3) δ: 1.71 (3H, s), 1.72 (3H, s), 2.27 (3H, s), 3.39 (3H, s), 3.58 (1H, dd, J=11.5, 9.5 Hz), 4.23 (1H, d d, J=9.5, 6.6 Hz), 4.70-4.76 (1H, m), 6.51-6.48 (2H, m), 6.69 (1H, d, J=7.3 Hz), 6.87 (1H, s), 6.92-6.97 (1H, m), 7.00-7.04 (1H, m), 7.06-7.14 (4H, m), 7.20 (1H, dd, J=8.8, 5.9 Hz), 7.78-7.81 (2H, m).

ESI-MS (m/e): 507 [M+H]+

### Example 11

### Synthesis of N-(2-{[(3R)-5-(3-chloro-5-cyanophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 1, using 3-bromo-5-chlorobenzonitrile.

The analytical data of the title compound are shown below.

¹H-NMR (CDCl₃) δ: 1.68 (3H, s,), 1.69 (3H, s,), 3.40 (3H, s), 3.56 (1H, dd, J=12.0, 8 Hz), 4.23 (1H, dd, J=8.0, 4 Hz), 4.73-4.79 (1H, m), 6.68 (1H, s), 6.75 (1H, s), 6.84 (1H, t, J=2 Hz), 7.02-7.14 (5H, m), 7.18-7.20 (2H, m), 7.80 (2H, dt, J=8.0, 4 Hz).

ESI-MS (m/e): 552 [M+H]+

### Example 12

### Synthesis of N-{1-[({(3R)-8-fluoro-1-methyl-2-oxo-5-[4-(trifluoromethoxy)phenyl]-2,3,4,5-tetrahydro-1H-1,5-benzodiazepin-3-yl}amino)carbonyl]cyclopropyl}benzamide

The title compound was obtained as a white solid by the method as in Example 1, using a compound obtained in Reference Example 5.

The analytical data of the title compound are shown below.

¹H-NMR (CDCl₃) δ: 1.08-1.26 (2H, m), 1.60-1.76 (2H, m), 3.35 (3H, s), 3.55 (1H, dd, J=12.0, 8 Hz), 4.17 (1H, dd, J=8.0, 4 Hz), 4.70-4.77 (1H, m), 6.64 (2H, d, J=9 Hz), 6.73 (1H, s), 6.93-7.04 (4H, m), 7.17 (1H, dd, J=8.8, 5.7 Hz), 7.45-7.57 (4H, m), 7.82 (2H, d, J=8 Hz).

ESI-MS (m/e): 557 [M+H]+

### Example 13

### Synthesis of N-{1-[({(3R)-5-[3,5-bis(trifluoromethyl)phenyl]-8-fluoro-1-methyl-2-oxo-2,3,4,5-tetrahydro-1H-1,5-benzodiazepin-3-yl}amino)carbonyl]cyclopropyl}benzamide

The title compound was obtained as a white solid by the method as in Example 1, using a compound obtained in Reference Example 5 and 1-bromo-3,5-bis(trifluoromethyl)benzene.

The analytical data of the title compound are shown below.

1H-NMR (CDCl3) δ: 1.13-1.18 (1H, m), 1.22-1.25 (1H, m), 1.65-1.67 (1H, m), 1.72-1.75 (1H, m), 3.37 (3H, s), 3.36-3.65 (1H, m), 4.19-4.23 (1H, m), 4.74-4.81 (1H, m), 6.73 (1H, s), 6.98-7.07 (4H, m), 7.18 (1H, dd, J=8.8, 5.9 Hz), 7.26-7.29 (1H, m), 7.52 (2H, t, J=6.8 Hz), 7.54-7.58 (2H, m), 7.83 (2H, d, J=6.8 Hz).

ESI-MS (m/e): 609 [M+H]+

### Example 14

### Synthesis of N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide

The title compound was obtained as a white solid by the method as in Example 1, using a compound obtained in Reference Example 4.

The analytical data of the title compound are shown below.

¹H-NMR (CDCl₃) δ: 1.69 (3H, s), 1.70 (3H, s), 3.39 (3H, s), 3.53-3.56 (1H, m), 4.23-4.27 (1H, m), 4.74-4.77 (1H, m), 6.67-6.69 (2H, m,), 6.77 (1H, s), 6.96-7.06 (4H, m), 7.18-7.22 (2H, m), 7.42-7.53 (3H, m), 7.77-7.79 (2H, m).

ESI-MS (m/e): 559 [M+H]+

### Example 15

### Synthesis of N-(2-{[(3R)-8-fluoro-1-methyl-2-oxo-5-phenyl-2,3,4,5-tetrahydro-1H-1,5-benzodiazepin-3-yl]amino}-1,1-dimethyl-2-oxoethyl)benzamide

The title compound was obtained as a colorless oily substance by the method as in Example 1, using the compound obtained in Reference Example 4 and bromobenzene.

The analytical data of the title compound are shown below.

¹H-NMR (CDCl₃) δ: 1.71 (6H, s), 3.40 (3H, s), 3.60 (1H, dd, J=12.0, 9.6 Hz), 4.26 (1H, dd, J=9.6, 6 Hz), 4.77 (1H, dt, J=12.0, 6 Hz), 6.71 (2H, d, J=7.8 Hz), 6.85-6.89 (2H, m), 6.93-6.96 (1H, m), 7.01-7.05 (1H, m), 7.18-7.23 (4H, m), 7.42-7.46 (2H, m), 7.49-7.53 (1H, m), 7.79 (2H, d, J=5.3 Hz).

ESI-MS (m/e): 475 [M+H]+

### Example 16

### Synthesis of N-(2-{[(3R)-8-fluoro-5-(4-fluorophenyl)-1-methyl-2-oxo-2,3,4,5-tetrahydro-1H-1,5-benzodiazepin-3-yl]amino}-1,1-dimethyl-2-oxoethyl)benzamide

The title compound was obtained as a colorless oily substance by the method as in Example 1, using the compound obtained in Reference Example 4 and 1-bromo-4-fluorobenzene.

The analytical data of the title compound are shown below.

¹H-NMR (CDCl₃) δ: 1.70 (6H, s), 3.41 (3H, s), 3.52 (1H, dd, J=12.0, 9.4 Hz), 4.26 (1H, dd, J=9.4, 6.2 Hz), 4.74 (1H, dt, J=12.0, 6.2 Hz), 6.65-6.69 (2H, m), 6.82 (1H, s), 6.89-6.97 (3H, m), 7.02 (1H, dd, J=9.2, 2.9 Hz), 7.13 (1H, dd, J=9.0, 5.9 Hz), 7.20 (1H, d, J=6.6 Hz), 7.42-7.53 (3H, m), 7.79 (2H, d, J=4.3 Hz).

ESI-MS (m/e): 493 [M+H]+

### Example 17

### Synthesis of N-[2-({(3R)-8-fluoro-1-methyl-2-oxo-5-[3-(trifluoromethoxy)phenyl]-2,3,4,5-tetrahydro-1H-1,5-benzodiazepin-3-yl}amino)-1,1-dimethyl-2-oxoethyl]benzamide

The title compound was obtained as a colorless oily substance by the method as in Example 5, using the compound obtained in Reference Example 4.

The analytical data of the title compound are shown below.

¹H-NMR (CDCl₃) δ: 1.70 (6H, s), 3.39 (3H, s), 3.58 (1H, dd, J=11.7, 9.7 Hz), 4.23 (1H, dd, J=9.7, 6.5 Hz), 4.73-4.80 (1H, m), 6.47 (1H, s), 6.60 (1H, dd, J=8.4, 2.2 Hz), 6.71 (1H, d, J=8.2 Hz), 6.79 (1H, brs), 6.96-7.01 (1H, m), 7.05 (1H, dd, J=12.0, 8 Hz), 7.16-7.24 (3H, m), 7.42-7.45 (2H, m), 7.49-7.53 (1H, m), 7.77-7.79 (2H, m).

ESI-MS (m/e): 559 [M+H]+

### Example 18

### Synthesis of N-[2-({(3R)-8-fluoro-1-methyl-2-oxo-5-[2-(trifluoromethoxy)phenyl]-2,3,4,5-tetrahydro-1H-1,5-benzodiazepin-3-yl}amino)-1,1-dimethyl-2-oxoethyl]benzamide

The title compound was obtained as a colorless oily substance by the method as in Example 1, using the compound obtained in Reference Example 4 and 1-bromo-2-(trifluoromethoxy)benzene.

The analytical data of the title compound are shown below.

¹H-NMR (CDCl₃) δ: 1.68 (3H, s), 1.69 (3H, s), 3.38-3.43 (4H, m), 4.46 (1H, dd, J=9.4, 6.2 Hz), 4.76 (1H, dt, J=12.9, 6.2 Hz), 6.77-6.86 (3H, m), 6.98 (1H, dd, J=9.0, 2.7 Hz), 7.01-7.05 (1H, m), 7.10-7.12 (1H, m), 7.20-7.30 (3H, m), 7.40-7.52 (3H, m), 7.76-7.78 (2H, m).

ESI-MS (m/e): 529 [M+H]+

### Example 19

### Synthesis of N-[2-({(3R)-5-[3,5-bis(trifluoromethyl)phenyl]-8-fluoro-1-methyl-2-oxo-2,3,4,5-tetrahydro-1H-1,5-benzodiazepin-3-yl}amino)-1,1-dimethyl-2-oxoethyl]-5-fluoropyridine-2-carboxyamide

The title compound was obtained as a white solid by the method as in Example 13, using the compound obtained in Reference Example 6.

The analytical data of the title compound are shown below.

1H-NMR (CDCl3) δ: 1.62 (6H, s), 3.09 (1H, dd, J=11.1, 9.6 Hz), 3.39 (3H, s), 3.55 (1H, dd, J=9.4, 7 Hz), 4.04 (1H, d, J=14.1 Hz), 4.31 (1H, d, J=14.1 Hz), 4.61-4.64 (1H, m), 6.93 (2H, t, J=5.1 Hz), 7.10 (4H, ddd, J=33.5, 13.2, 6.5 Hz), 7.25 (2H, d, J=8.6 Hz), 7.51 (1H, td, J=8.4, 2.7 Hz), 8.13 (1H, dd, J=8.8, 4.5 Hz), 8.30 (1H, s), 8.37 (1H, d, J=2.7 Hz).

ESI-MS (m/e): 563.4 [M+H]+

### Example 20

### Synthesis of N-(2-{[(3R)-5-(3,5-difluorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridine-2-carboxyamide

The title compound was obtained as a white solid by the method as in Example 2, using the compound obtained in Reference Example 6.

The analytical data of the title compound are shown below.

1H-NMR (CDCl3) δ: 1.63 (3H, s), 1.64 (3H, s), 3.32 (3H, s), 3.50 (1H, dd, J=11.7, 9.8 Hz), 4.08 (1H, q, J=7.2 Hz), 4.72 (1H, dt, J=12.6, 5.8 Hz), 6.09 (2H, dd, J=10.0, 2.2 Hz), 6.24 (1H, tt, J=8.8, 2.2 Hz), 6.94-7.02 (2H, m), 7.20 (1H, d d, J=9.0, 5.5 Hz), 7.29 (1H, d, J=6.6 Hz), 7.51 (1H, td, J=8.4, 2.9 Hz), 8.19 (1H, dd, J=8.6, 4.7 Hz), 8.26 (1H, s), 8.36 (1H, d, J=2.7 Hz).

ESI-MS (m/e): 507.4 [M+H]+

### Example 21

### Synthesis of N-(2-{[(3R)-5-(3-chloro-5-fluorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridine-2-carboxyamide

The title compound was obtained as a white solid by the method as in Example 1, using the compound obtained in Reference Example 6 and 1-bromo-3-chloro-5-fluorobenzene.

The analytical data of the title compound are shown below.

1H-NMR (CDCl3) δ: 1.67 (3H, s), 1.70 (3H, t, J=8.4 Hz), 3.37 (3H, s), 3.54 (1H, dd, J=11.7, 9.8 Hz), 4.12-4.18 (1H, m), 4.76 (1H, dt, J=12.4, 5.9 Hz), 6.24 (1H, dt, J=11.3, 2.2 Hz), 6.40 (1H, s), 6.56 (1H, dt, J=8.2, 1.8 Hz), 6.98-7.06 (2H, m), 7.22 (1H, dd, J=8.8, 5.7 Hz), 7.33 (1H, d, J=6.6 Hz), 7.55 (1H, td, J=8.3, 2.9 Hz), 8.23 (1H, dd, J=8.8, 4.5 Hz), 8.31 (1H, s), 8.40 (1H, d, J=2.7 Hz).

ESI-MS (m/e): 557.4 [M+H]+

### Example 22

### Synthesis of N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-2-fluoroisonicotinamide

The title compound was obtained as a white solid by the method as in Example 3, using the compound obtained in Reference Example 7.

The analytical data of the title compound are shown below.

1H-NMR (CDCl3) δ: 1.68 (3H, s), 1.71 (3H, s), 3.38 (3H, s), 3.52 (1H, dd, J=11.3, 9.8 Hz), 4.14 (1H, dd, J=9.6, 6.5 Hz), 4.65-4.72 (1H, m), 6.47 (2H, d, J=2 Hz), 6.81 (1H, t, J=1.6 Hz), 6.98-7.09 (3H, m), 7.13 (1H, s), 7.18-7.20 (1H, m), 7.24-7.26 (1H, m), 7.46 (1H, td, J=3.2, 1.7 Hz), 8.29 (1H, d, J=5.1 Hz).

ESI-MS (m/e): 562 [M+H]+

### Example 23

### Synthesis of N-(2-{[(3R)-8-chloro-5-(3,5-dichlorophenyl)-1-methyl-2-oxo-2,3,4,5-tetrahydro-1H-1,5-benzodiazepin-3-yl]amino}-1,1-dimethyl-2-oxoethyl)-5-fluoropyridine-2-carboxyamide

The title compound was obtained as a white solid by the method as in Example 3, using the compound obtained in Reference Example 19.

The analytical data of the title compound are shown below.

1H-NMR (CDCl3) δ: 1.67 (3H, s), 1.68 (3H, s), 3.38 (3H, s), 3.48-3.53 (1H, m), 4.17-4.21 (1H, m), 4.73-4.76 (1H, m), 6.54 (2H, d, J=1.7 Hz), 6.84 (1H, t, J=1.7 Hz), 7.16 (1H, d, J=8.8 Hz), 7.24-7.32 (3H, m), 7.52-7.57 (1H, m), 8.21-8.25 (1H, m), 8.29 (1H, s), 8.40 (1H, d, J=2.9 Hz).

ESI-MS (m/e): 578 [M+H]+

### Example 24

### Synthesis of N-(2-{[(3R)-8-chloro-5-(3,5-difluorophenyl)-1-methyl-2-oxo-2,3,4,5-tetrahydro-1H-1,5-benzodiazepin-3-yl]amino}-1,1-dimethyl-2-oxoethyl)-5-fluoropyridine-2-carboxyamide

The title compound was obtained as a white solid by the method as in Example 2, using the compound obtained in Reference Example 19.

The analytical data of the title compound are shown below.

1H-NMR (CDCl3) δ: 1.67 (3H, s), 1.68 (3H, s), 3.37 (3H, s), 3.49-3.55 (1H, m), 4.11-4.20 (1H, m), 4.74-4.77 (1H, m), 6.15-6.18 (2H, m), 6.27-6.30 (1H, m), 7.19-7.32 (4H, m), 7.52-7.57 (1H, m), 8.21-8.24 (1H, m), 8.29 (1H, s), 8.40 (1H, d, J=2.4 Hz).

ESI-MS (m/e): 546 [M+H]+

### Example 25

### Synthesis of N-(2-{[(3R)-8-chloro-5-(3,5-dichlorophenyl)-1-methyl-2-oxo-2,3,4,5-tetrahydro-1H-1,5-benzodiazepin-3-yl]amino}-1,1-dimethyl-2-oxoethyl)-2-fluoroisonicotinamide

The title compound was obtained as a colorless oily substance by the method as in Example 3, using the compound obtained in Reference Example 20.

The analytical data of the title compound are shown below.

¹H-NMR (CDCl₃) δ: 1.76 (3H, s), 1.80 (3H, s), 3.47 (3H, s), 3.59 (1H, dd, J=12.0, 8 Hz), 4.19 (1H, dd, J=12.0, 8 Hz), 4.73-4.79 (1H, m), 6.59 (2H, s), 6.91 (1H, s), 7.12-7.24 (3H, m), 7.31-7.39 (3H, m), 7.53-7.55 (1H, m), 8.38 (1H, d, J=5.1 Hz).

ESI-MS (m/e): 578, 580 [M+H]+

### Example 26

### Synthesis of N-(2-{[(3R)-8-chloro-5-(3,5-difluorophenyl)-1-methyl-2-oxo-2,3,4,5-tetrahydro-1H-1,5-benzodiazepin-3-yl]amino}-1,1-dimethyl-2-oxoethyl)-2-fluoroisonicotinamide

The title compound was obtained as a colorless oily substance by the method as in Example 2, using the compound obtained in Reference Example 20.

The analytical data of the title compound are shown below.

¹H-NMR (CDCl₃) δ: 1.71 (3H, s), 1.74 (3H, s), 3.41 (3H, s), 3.56 (1H, dd, J=11.5, 8.8 Hz), 4.18 (1H, dd, J=8.8, 4 Hz), 4.73 (1H, dt, J=12.0, 8 Hz), 6.16-6.19 (2H, m), 6.32 (1H, t, J=8.8 Hz), 7.09-7.15 (2H, m), 7.22 (1H, d, J=8 Hz), 7.25-7.30 (2H, m), 7.34 (1H, d, J=4 Hz), 7.50 (1H, td, J=3.4, 1.7 Hz), 8.33 (1H, d, J=5.1 Hz).

ESI-MS (m/e): 546 [M+H]+

### Example 27

### Synthesis of N-{(3R)-5-[3,5-bis(trifluoromethyl)phenyl]-8-fluoro-1-methyl-2-oxo-2,3,4,5-tetrahydro-1H-1,5-benzodiazepin-3-yl}-2-methyl-N²-{[1-(trifluoromethyl)cyclopropyl]carbonyl}alaninamide

The title compound was obtained as a white solid by the method as in Example 13, using the compound obtained in Reference Example 8.

The analytical data of the title compound are shown below.

¹H-NMR (CDCl₃) δ: 1.25-1.48 (4H, m), 1.64 (3H, s), 1.66 (3H, s), 3.46 (3H, s), 3.60 (1H, dd, J=11.7, 9.7 Hz), 4.29 (1H, dd, J=9.7, 6.5 Hz), 4.76-4.82 (1H, m), 6.79 (1H, s), 7.04-7.11 (4H, m), 7.15 (1H, dd, J=9.0, 2.7 Hz), 7.24 (1H, d d, J=9.0, 5.7 Hz), 7.37 (1H, s).

ESI-MS (m/e): 643 [M+H]+

### Example 28

### Synthesis of N-[(3R)-8-chloro-5-(3,5-dichlorophenyl)-1-methyl-2-oxo-2,3,4,5-tetrahydro-1H-1,5-benzodiazepin-3-yl]-2-methyl-N²-{[1-(trifluoromethyl)cyclopropyl]carbonyl}alaninamide

The title compound was obtained as a white solid by the method as in Example 3, using the compound obtained in Reference Example 21.

The analytical data of the title compound are shown below.

1H-NMR (CDCl3) δ: 1.21-1.24 (2H, m), 1.52-1.58 (2H, m), 1.59 (3H, s), 1.60 (3H, s), 3.40 (3H, s), 3.44-3.50 (1H, m), 4.11-4.17 (1H, m), 4.67-4.70 (1H, m), 6.53 (2H, d, J=1.8 Hz), 6.78 (1H, s), 6.85 (1H, t, J=1.8 Hz), 7.00-7.02 (1H, m), 7.16 (1H, d, J=8.8 Hz), 7.23-7.29 (1H, m), 7.32 (1H, d, J=2.4 Hz).

ESI-MS (m/e): 591 [M+H]+

### Example 29

### Synthesis of N-[(3R)-8-chloro-5-(3,5-difluorophenyl)-1-methyl-2-oxo-2,3,4,5-tetrahydro-1H-1,5-benzodiazepin-3-yl]-2-methyl-N²-{[1-(trifluoromethyl) cyclopropyl]carbonyl}alaninamide

The title compound was obtained as a white solid by the method as in Example 2, using the compound obtained in Reference Example 21.

The analytical data of the title compound are shown below.

1H-NMR (CDCl3) δ: 1.22-1.25 (2H, m), 1.40-1.50 (2H, m), 1.58 (3H, s), 1.59 (3H, s), 3.38 (3H, s), 3.47-3.53 (1H, m), 4.11-4.15 (1H, m), 4.66-4.72 (1H, m), 6.28-6.29 (2H, m), 6.30-6.34 (1H, m), 6.76 (1H, s), 7.13 (1H, s), 7.20 (1 H, d, J=8.8 Hz), 7.32 (1H, d, J=2 Hz), 7.42-7.44 (1H, m).

ESI-MS (m/e): 559 [M+H]+

### Example 30

### Synthesis of N-[2-({(3R))-5-[3,5-bis(trifluoromethyl)phenyl]-1-methyl-2-oxo-2,3,4,5-tetrahydro-1H-1,5-benzodiazepin-3-yl}amino)-1,1-dimethyl-2-oxoethyl]-4-fluorobenzamide

The title compound was obtained as a white solid by the method as in Example 13, using the compound obtained in Reference Example 12.

The analytical data of the title compound are shown below.

1H-NMR (CDCl3) δ: 1.70 (3H, s), 1.72 (3H, s), 3.44 (3H, s), 3.58-3.63 (1H, m), 4.29-4.33 (1H, m), 4.75-4.81 (1H, m), 6.74 (1H, s), 7.00 (2H, m), 7.05-7.13 (2H, m), 7.19-7.25 (2H, m), 7.25-7.42 (2H, m), 7.37-7.42 (2H, m), 7.78-7.82 (2H, m).

ESI-MS (m/e): 611 [M+H]+

### Example 31

### Synthesis of N-[2-({(3S)-5-[3,5-bis(trifluoromethyl)phenyl]-1-methyl-2-oxo-2,3,4,5-tetrahydro-1H-1,5-benzodiazepin-3-yl}amino)-1,1-dimethyl-2-oxoethyl]-4-fluorobenzamide

The title compound was obtained as a white solid by the method as in Example 30, using 3-amino-N-(tert-butoxycarbonyl)-L-alanine in the synthesis described in Reference Example 9.

### Example 32

### Synthesis of N-[1,1-dimethyl-2-({(3R)-1-methyl-2-oxo-5-[4-(trifluoromethoxy)phenyl]-2,3,4,5-tetrahydro-1H-1,5-benzodiazepin-3-yl}amino)-2-oxoethyl]-4-fluorobenzamide

The title compound was obtained as a white solid by the method as in Example 1, using the compound obtained in Reference Example 12.

The analytical data of the title compound are shown below.

1H-NMR (CDCl3) δ: 1.70 (3H, s), 1.71 (3H, s), 3.41 (3H, s), 3.53-3.58 (1H, m), 4.24-4.28 (1H, m), 4.71-4.77 (1H, m), 6.71 (2H, d, J=9.3 Hz), 6.81 (1H, s), 7.04-7.18 (5H, m), 7.21-7.31 (4H, m), 7.79 (2H, dd, J=8.8, 4.9 Hz).

ESI-MS (m/e): 559 [M+H]+

### Example 33

### Synthesis of N-[1,1-dimethyl-2-({(3S)-1-methyl-2-oxo-5-[4-(trifluoromethoxy)phenyl]-2,3,4,5-tetrahydro-1H-1,5-benzodiazepin-3-yl}amino)-2-oxoethyl]-4-fluorobenzamide

The title compound was obtained as a white solid by the method as in Example 32, using 3-amino-N-(tert-butoxycarbonyl)-L-alanine in the synthesis described in Reference Example 9.

### Example 34

### Synthesis of N-(2-{[(3R)-5-(3,5-dichlorophenyl)-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 3, using the compound obtained in Reference Example 12.

The analytical data of the title compound are shown below.

1H-NMR (CDCl3) δ: 1.69 (3H, s), 1.71 (3H, s), 3.41 (3H, s), 3.54 (1H, dd, J=11.3, 9.8 Hz), 4.20 (1H, dd, J=9.6, 6.5 Hz), 4.70-4.76 (1H, m), 6.55 (2H, d, J=1.6 Hz), 6.81-6.82 (2H, m), 7.08-7.15 (2H, m), 7.18-7.38 (5H, m), 7.79-7.80 (2H, m).

ESI-MS (m/e): 543 [M+H]+

### Example 35

### Synthesis of N-(2-{[(3S)-5-(3,5-dichlorophenyl)-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 34, using 3-amino-N-(tert-butoxycarbonyl)-L-alanine in the synthesis described in Reference Example 9.

### Example 36

### Synthesis of N-{1-[({(3R)-8-chloro-1-methyl-2-oxo-5-[4-(trifluoromethoxy)phenyl]-2,3,4,5-tetrahydro-1H-1,5-benzodiazepin-3-yl}amino)carbonyl]cyclopropyl}benzamide

The title compound was obtained as a white solid by the method as in Example 1, using the compound obtained in Reference Example 17.

The analytical data of the title compound are shown below.

¹H-NMR (DMSO-D₆) δ: 0.96-1.06 (2H, m), 1.25-1.31 (2H, m), 3.25 (3H, s), 3.75-3.85 (2H, m), 4.53 (1H, dt, J=13.7, 5.8 Hz), 6.70-6.73 (2H, m), 7.14-7.18 (3H, m), 7.33 (1H, dd, J=8.7, 2.3 Hz), 7.43-7.55 (3H, m), 7.70 (1H, d, J=2.3 Hz), 7.88-7.90 (3H, m), 8.98 (1H, s).

ESI-MS (m/e): 573 [M+H]+

### Example 37

### Synthesis of N-{1-[({(3R)-5-[3,5-bis(trifluoromethyl)phenyl]-8-chloro-1-methyl-2-oxo-2,3,4,5-tetrahydro-1H-1,5-benzodiazepin-3-yl}amino)carbonyl]cyclopropyl}benzamide

The title compound was obtained as a white solid by the method as in Example 13, using the compound obtained in Reference Example 17.

The analytical data of the title compound are shown below.

1H-NMR (CDCl3) δ: 1.15-1.18 (1H, m), 1.22-1.29 (1H, m), 1.62-1.67 (1H, m), 1.72-1.78 (1H, m), 3.37 (3H, s), 3.59-3.64 (1H, m), 4.19-4.23 (1H, m), 4.74-4.80 (1H, m), 6.74 (1H, s), 7.01 (2H, s), 7.14 (1H, d, J=8.3 Hz), 7.25-7.33 (3H, m), 7.48 (2H, d, J=7.4 Hz), 7.54-7.58 (2H, m), 7.82 (2H, d, J=7.4 Hz).

ESI-MS (m/e): 625 [M+H]+

### Example 38

### Synthesis of N-(2-{[(3R)-8-chloro-5-(3,5-dichlorophenyl)-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a pale yellow solid by the method as in Example 3, using the compound obtained in Reference Example 18.

The analytical data of the title compound are shown below.

1H-NMR (CDCl3) δ: 1.69 (3H, s), 1.70 (3H, s), 3.40 (3H, s), 3.53 (1H, dd, J=11.7, 9.8 Hz), 4.19 (1H, dd, J=9.8, 6.3 Hz), 4.69-4.75 (1H, m), 6.55 (2H, d, J=1.5 Hz), 6.74 (1H, s), 6.86-6.85 (1H, m), 7.09-7.13 (2H, m), 7.16-7.18 (2H, m), 7.25-7.27 (1H, m), 7.33 (1H, d, J=2 Hz), 7.78-7.82 (2H, m).

ESI-MS (m/e): 577 [M+H]+

### Example 39

### Synthesis of N-[2-({(3R)-5-[3,5-bis(trifluoromethyl)phenyl]-8-chloro-1-methyl-2-oxo-2,3,4,5-tetrahydro-1H-1,5-benzodiazepin-3-yl}amino)-1,1-dimethyl-2-oxoethyl]-4-fluorobenzamide

The title compound was obtained as a white solid by the method as in Example 13, using the compound obtained in Reference Example 18.

The analytical data of the title compound are shown below.

1H-NMR (CDCl3) δ: 1.69 (3H, s), 1.71 (3H, s), 3.42 (3H, s), 3.57-3.62 (1H, m), 4.27-4.31 (1H, m), 4.75-4.81 (1H, m), 6.67 (1H, s), 7.04-7.06 (2H, m), 7.10-7.20 (4H, m), 7.25-7.29 (1H, m), 7.32-7.35 (1H, m), 7.37-7.38 (1H, m), 7.78-7.82 (2H, m).

ESI-MS (m/e): 645 [M+H]+

### Example 40

### Synthesis of N-[2-({(3R)-8-chloro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]-4-fluorobenzamide

The title compound was obtained as a white solid by the method as in Example 1, using the compound obtained in Reference Example 18.

The analytical data of the title compound are shown below.

1H-NMR (CDCl3) δ: 1.69 (3H, s), 1.70 (3H, s), 3.40 (3H, s), 3.51-3.57 (1H, m), 4.23-4.27 (1H, m), 4.72-4.75 (1H, m), 7.00-6.74 (3H, m), 7.05-7.17 (6H, m), 7.20-7.31 (2H, m), 7.79 (2H, dd, J=8.8, 5.4 Hz).

ESI-MS (m/e): 593 [M+H]+

### Example 41

### Synthesis of N-(2-{[(3R)-8-chloro-5-(3,5-difluorophenyl)-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 2, using the compound obtained in Reference Example 18.

The analytical data of the title compound are shown below.

1H-NMR (CDCl3) δ: 1.68 (6H, s), 3.39 (3H, s), 3.54 (1H, dd, J=11.7, 9.8 Hz), 4.17 (1H, dd, J=9.8, 6.3 Hz), 4.74 (1H, dt, J=11.7, 6.3 Hz), 6.17 (2H, dd, J=9.8, 2 Hz), 6.30 (1H, tt, J=9.3, 2 Hz), 6.77 (1H, brs), 7.10 (1H, t, J=9.3 Hz), 7.18-7.33 (5H, m), 7.75-7.83 (2H, m).

ESI-MS (m/e): 545 [M+H]+

### Example 42

### Synthesis of N-(2-{[(3R)-8-chloro-5-(3,4-dichlorophenyl)-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 9, using the compound obtained in Reference Example 18.

The analytical data of the title compound are shown below.

1H-NMR (CDCl3) δ: 1.69 (6H, s), 3.40 (3H, s), 3.51 (1H, dd, J=11.7, 9.8 Hz), 4.20 (1H, dd, J=9.8, 6.3 Hz), 4.72 (1H, dt, J=11.7, 6.3 Hz), 6.54 (1H, dd, J=8.8, 2.9 Hz), 6.76 (1H, brs), 6.78 (1H, d, J=2.9 Hz), 7.06-7.34 (7H, m), 7.76-7.83 (2H, m).

ESI-MS (m/e): 578 [M+H]+

### Example 43

### Synthesis of N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a pale yellow solid by the method as in Reference Example 3, using the compound obtained in Reference Example 30 and 4-fluorobenzoic acid.

The analytical data of the title compound are shown below.

¹H-NMR (CDCl₃) δ: 1.69 (3H, s), 1.70 (3H, s), 3.40 (3H, s), 3.54 (1H, dd, J=11.3, 9.8 Hz), 4.19 (1H, dd, J=9.8, 6.3 Hz), 4.72 (1H, dt, J=11.3, 6.3 Hz), 6.51-6.53 (2H, m), 6.73 (1H, s), 6.83-6.84 (1H, m), 7.00-7.23 (6H, m), 7.78-7.82 (2H, m).

ESI-MS (m/e): 561 [M+H]+

### Example 44

### Synthesis of N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridine-2-carboxyamide

The title compound was obtained as a white solid by the method as in Reference Example 3, using the compound obtained in Reference Example 30 and 5-fluoropyridine-2-carboxylic acid.

The analytical data of the title compound are shown below.

1H-NMR (CDCl3) δ: 1.67 (3H, s), 1.68 (3H, s), 3.37 (3H, s), 3.52 (1H, dd, J=11.7, 9.8 Hz), 4.19 (1H, dd, J=9.8, 6.3 Hz), 4.72-4.78 (1H, m), 6.50-6.51 (2H, m), 6.82-6.82 (1H, m), 6.98-7.06 (2H, m), 7.20 (1H, dd, J=9.0, 5.6 Hz), 7.32 (1H, d, J=6.8 Hz), 7.54 (1H, td, J=8.4, 2.8 Hz), 8.23 (1H, dd, J=8.8, 4.9 Hz), 8.30 (1H, brs), 8.40 (1H, d, J=2.4 Hz).

LCMS (m/z): 562 [M+H]+

### Example 45

### Synthesis of N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-1,3-thiazol-2-carboxyamide

The title compound was obtained as a white solid by the method as in Reference Example 3, using the compound obtained in Reference Example 30 and 1,3-thiazol-2-carboxylic acid.

The analytical data of the title compound are shown below.

1H-NMR (DMSO-D6) δ: 0.81-1.37 (4H, m), 1.99-2.34 (3H, m), 2.93-3.06 (1H, brm), 4.12-4.20 (1H, brm), 5.08 (1H, d, J=15.6 Hz), 5.25 (1H, d, J=15.6 Hz), 7.24-7.54 (6H, m), 7.82-7.98 (6H, m), 8.90-8.86 (1H, brm).

ESI-MS (m/z): 624 [M+H]+

### Example 46

### Synthesis of N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-(difluoromethoxy)benzamide

The title compound was obtained as a white solid by the method as in Reference Example 3, using the compound obtained in Reference Example 30 and 4-(difluoromethoxy)benzoic acid.

The analytical data of the title compound are shown below.

¹H-NMR (CDCl₃) δ: 1.69 (3H, s), 1.70 (3H, s), 3.39 (3H, s), 3.54 (1H, dd, J=11.5, 9.8 Hz), 4.18 (1H, dd, J=9.8, 6.5 Hz), 4.70-4.76 (1H, m), 6.51-6.52 (2H, m), 6.65 (1H, t, J=73.1 Hz), 6.77 (1H, s), 6.83 (1H, s), 7.00-7.07 (2H, m), 7.15-7.23 (5H, m), 7.81 (2H, d, J=8 Hz).

ESI-MS (m/e): 609 [M+H]+

### Example 47

### Synthesis of N²-[(tert-butylamino)carbonyl]-N-[(3R)-5-(3,5-dichlorophenyl)-8-fluoro-1-methyl-2-oxo-2,3,4,5-tetrahydro-1H-1,5-benzodiazepin-3-yl]-2-methylalanineamide

To a solution of compound, obtained in Reference Example 30, in chloroform, tert-butyl isocyanate was added at room temperature, and the mixture was stirred at 55° C. for 15 hours. The residue obtained by removing the solvent under reduced pressure was purifies by column chromatography (hexane-ethyl acetate) to yield the title compound as a white solid.

The analytical data of the title compound are shown below.

1H-NMR (CDCl3) δ: 1.35 (9H, s), 1.44 (3H, s), 1.49 (3H, s), 3.38 (3H, s), 3.50 (1H, dd, J=11.5, 10 Hz), 4.10-4.17 (1H, m), 4.30 (1H, s), 4.45 (1H, s), 4.72-4.75 (1H, m), 6.50-6.51 (2H, m), 6.80-6.81 (1H, m), 6.98-7.05 (2H, m), 7.20 (1H, dd, J=8.8, 5.9 Hz), 7.26-7.28 (1H, m).

LCMS (m/z): 38 [M+H]+

### Example 48

### Synthesis of N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-(trifluoromethoxy)benzamide

The title compound was obtained as a white solid by the method as in Reference Example 3, using the compound obtained in Reference Example 30 and 4-(trifluoromethoxy)benzoic acid.

The analytical data of the title compound are shown below.

¹H-NMR (CDCl₃) δ: 1.70 (3H, s), 1.71 (3H, s), 3.40 (3H, s), 3.55 (1H, dd, J=12.0, 8.8 Hz), 4.19 (1H, dd, J=8.8, 6.5 Hz), 4.70-4.76 (1H, m), 6.51 (2H, s), 6.80-6.84 (2H, m), 7.00-7.07 (2H, m), 7.17-7.23 (2H, m), 7.26-7.28 (2H, m), 7.83 (2H, d, J=9 Hz).

ESI-MS (m/e): 627 [M+H]+

### Example 49

### Synthesis of N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-3,4-difluorobenzamide

The title compound was obtained as a white solid by the method as in Reference Example 3, using the compound obtained in Reference Example 30 and 3,4-difluorobenzoic acid.

The analytical data of the title compound are shown below.

¹H-NMR (CDCl₃) δ: 1.69 (3H, s), 1.71 (3H, s), 3.40 (3H, s), 3.55 (1H, dd, J=11.7, 9.8 Hz), 4.19 (1H, dd, J=9.8, 6.3 Hz), 4.69-4.75 (1H, m), 6.51 (2H, d, J=2 Hz), 6.82-6.84 (2H, m), 7.00-7.08 (2H, m), 7.13 (1H, d, J=6.3 Hz), 7.18-7.25 (2H, m), 7.51-7.53 (1H, m), 7.63-7.68 (1H, m).

ESI-MS (m/e): 579 [M+H]+

### Example 50

### Synthesis of N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]-1-methyl-1H-imidazol-2-carboxyamide

The title compound was obtained as a white solid by the method as in Reference Example 3, using the compound obtained in Reference Example 31 and 1-methyl-1H-imidazol-2-carboxylic acid.

The analytical data of the title compound are shown below.

1H-NMR (CDCl3) δ: 1.61 (3H, s), 1.62 (3H, s), 3.36 (3H, d, J=10.6 Hz), 3.53 (1H, dd, J=11.5, 9.6 Hz), 4.04 (3H, s), 4.23 (1H, dd, J=9.6, 6.5 Hz), 4.76 (1H, dt, J=12.5, 5.8 Hz), 6.66 (2H, td, J=6.5, 3.9 Hz), 6.96 (2H, dq, J=9.4, 2.9 Hz), 7.01-7.05 (4H, m), 7.18 (1H, dd, J=9.0, 5.9 Hz), 7.32 (1H, d, J=7 Hz), 7.67 (1H, s).

ESI-MS (m/e): 563.4 [M+H]+

### Example 51

### Synthesis of 4-(difluoromethoxy)-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide

The title compound was obtained as a colorless solid by the method as in Reference Example 3, using the compound obtained in Reference Example 31 and 4-(difluoromethoxy)benzoic acid.

The analytical data of the title compound are shown below.

1H-NMR (DMSO-D6) δ: 1.39 (3H, s), 1.40 (3H, s), 3.21 (3H, s), 3.68-3.79 (2H, m), 4.43-4.52 (1H, m), 6.60-6.64 (2H, m), 7.09-7.23 (6H, m), 7.30 (1H, t, J=73.5 Hz), 7.51 (1H, dd, J=9.8, 2.7 Hz), 7.70 (1H, d, J=7.8 Hz), 7.90-7.85 (2H, m), 8.30 (1H, s).

ESI-MS (m/z): 625 [M+H]+

### Example 52

### Synthesis of N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]piperidin-1-carboxyamide

The title compound was obtained by the method as in Reference Example 3, using the compound obtained in Reference Example 31 and piperidin-1-carboxylic acid.

The analytical data of the title compound are shown below.

1H-NMR (DMSO-D6) δ: 1.35 (3H, s), 1.37 (3H, s), 1.48 (4H, s), 1.57 (2H, d, J=4.3 Hz), 3.31 (7H, t, J=6.8 Hz), 3.71 (1H, t, J=10.9 Hz), 3.86 (1H, dd, J=9.8, 6.6 Hz), 4.49 (1H, dd, J=12.9, 5.5 Hz), 6.43 (1H, s), 6.73 (2H, d, J=9 Hz), 7.20-7.32 (4H, m), 7.46 (1H, d, J=7.8 Hz), 7.60 (1H, dd, J=10.0, 2.9 Hz).

ESI-MS (m/e): 566.4 [M+H]+

### Example 53

### Synthesis of N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]-1,3-thiazol-2-carboxyamide

The title compound was obtained as a colorless solid by the method as in Reference Example 3, using the compound obtained in Reference Example 31 and 1,3-thiazol-2-carboxylic acid.

The analytical data of the title compound are shown below.

1H-NMR (DMSO-D6) δ: 1.60 (6H, d, J=1.2 Hz), 3.32 (3H, s), 3.81-3.88 (1H, m), 3.94 (1H, t, J=11.1 Hz), 4.62 (1H, dt, J=13.9, 6.1 Hz), 6.74 (2H, d, J=9.4 Hz), 7.20-7.30 (4H, m), 7.61 (1H, dd, J=9.8, 2.7 Hz), 8.07 (2H, dd, J=12.9, 3.1 Hz), 8.19 (1H, d, J=8.2 Hz), 8.64 (1H, s).

ESI-MS (m/e): 566.3 [M+H]+

### Example 54

### Synthesis of N-{(3R)-8-fluoro-1-methyl-2-oxo-5-[4-(trifluoromethoxy)phenyl]-2,3,4,5-tetrahydro-1H-1,5-benzodiazepin-3-yl}-2-methyl-N²-{[1-(trifluoromethyl)cyclopropyl]carbonyl}alaninamide

The title compound was obtained as a white solid by the method as in Reference Example 3, using the compound obtained in Reference Example 31 and 1-(trifluoromethyl)cyclopropanecarboxylic acid.

The analytical data of the title compound are shown below.

1H-NMR (DMSO-D6) δ: 1.22 (2H, t, J=7.2 Hz), 1.43-1.44 (8H, m), 3.32 (3H, s), 3.82 (2H, dt, J=28.0, 9.6 Hz), 4.55 (1H, dd, J=13.5, 6.5 Hz), 6.74 (2H, d, J=9 Hz), 7.22-7.30 (4H, m), 7.61 (1H, t, J=6.1 Hz), 7.89 (2H, d, J=5.9 Hz).

ESI-MS (m/e): 591.3 [M+H]+

### Example 55

### Synthesis of 4-fluoro-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]-2-(trifluoromethyl)benzamide

The title compound was obtained as a colorless solid by the method as in Reference Example 3, using the compound obtained in Reference Example 31 and 4-fluoro-2-(trifluoromethyl)benzoic acid.

The analytical data of the title compound are shown below.

1H-NMR (DMSO-D6) δ: 1.34 (3H, s), 1.35 (3H, s), 3.25 (3H, s), 3.73 (1H, dd, J=10.2, 6.6 Hz), 3.82 (1H, dd, J=11.7, 10.2 Hz), 4.45-4.55 (1H, m), 6.63-6.66 (2H, m), 7.10-7.23 (4H, m), 7.52 (1H, dd, J=10.0, 2.9 Hz), 7.60-7.68 (2H, m), 7.75 (1H, d, J=7.8 Hz), 7.84 (1H, dd, J=8.4, 5.7 Hz), 8.67 (1H, s).

ESI-MS (m/z): 645 [M+H]+

### Example 56

### Synthesis of 6-fluoro-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]pyridine-2-carboxyamide

The title compound was obtained as a white solid by the method as in Reference Example 3, using the compound obtained in Reference Example 31 and 6-fluoropyridine-2-carboxylic acid. The analytical data of the title compound are shown below.

1H-NMR (CDCl3) δ: 1.67 (3H, s), 1.68 (3H, s), 3.38 (3H, d, J=4.7 Hz), 3.56 (1H, dd, J=11.3, 9.8 Hz), 4.24 (1H, dd, J=9.6, 6.5 Hz), 4.76 (1H, dt, J=12.5, 5.8 Hz), 6.67 (2H, dt, J=9.9, 2.8 Hz), 6.97 (1H, ddd, J=9.7, 6.9, 2.1 Hz), 7.04 (3H, td, J=6.0, 3.1 Hz), 7.12 (1H, dd, J=8.2, 2.7 Hz), 7.19 (1H, dd, J=9.0, 5.9 Hz), 7.34 (1H, d, J=6.6 Hz), 7.97 (1H, q, J=7.8 Hz), 8.07 (1H, dd, J=7.4, 2 Hz), 8.14 (1H, s).

ESI-MS (m/e): 578.3 [M+H]+

### Example 57

### Synthesis of 5,6-difluoro-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]pyridine-2-carboxyamide

The title compound was obtained as a white solid by the method as in Reference Example 3, using the compound obtained in Reference Example 31 and 5,6-difluoropyridine-2-carboxylic acid.

The analytical data of the title compound are shown below.

1H-NMR (CDCl3) δ: 1.68 (6H, s), 3.38 (3H, d, J=4.3 Hz), 3.55 (1H, t, J=10.6 Hz), 4.24 (1H, dd, J=9.4, 6.6 Hz), 4.75 (1H, t, J=5.9 Hz), 6.66 (2H, t, J=4.5 Hz), 6.98 (1H, td, J=8.2, 3 Hz), 7.04 (3H, td, J=6.1, 3 Hz), 7.19 (1H, dd, J=9.0, 5.9 Hz), 7.29 (2H, t, J=8.8 Hz), 7.72 (1H, t, J=8.6 Hz), 7.99 (1H, s), 8.08 (1H, dd, J=8.2, 3.1 Hz).

ESI-MS (m/e): 596.3 [M+H]+

### Example 58

### Synthesis of 3-chloro-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]thiophene-2-carboxyamide

The title compound was obtained as a white solid by the method as in Reference Example 3, using the compound obtained in Reference Example 31 and 3-chlorothiophen-2-carboxylic acid.

The analytical data of the title compound are shown below.

1H-NMR (CDCl3) δ: 1.70 (6H, d, J=0.8 Hz), 3.39 (3H, s), 3.57 (1H, dd, J=11.3, 9.8 Hz), 4.24 (1H, dd, J=9.8, 6.6 Hz), 4.73-4.78 (1H, m), 6.67 (2H, dd, J=7.0, 2.3 Hz), 6.97 (2H, dq, J=10.8, 2.9 Hz), 7.05 (3H, td, J=5.8, 2.9 Hz), 7.19 (2H, dd, J=8.8, 5.7 Hz), 7.48 (1H, t, J=6.1 Hz), 7.69 (1H, s).

ESI-MS (m/e): 594.3 [M+H]+

### Example 59

### Synthesis of N-(2-{[(3R)-5-(3,5-dichlorophenyl)-7-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 3, using the compound obtained in Reference Example 32.

The analytical data of the title compound are shown below.

1H-NMR (CDCl3) δ: 7.82-7.78 (2H, m), 7.31 (1H, dd, J=9.0, 5.4 Hz), 7.17 (1H, d, J=6.3 Hz), 7.14-7.05 (3H, m), 6.95 (1H, dd, J=9.0, 2.9 Hz), 6.89 (1H, t, J=2 Hz), 6.73 (1H, s), 6.60 (2H, d, J=2 Hz), 4.72 (1H, dt, J=11.2, 6.3 Hz), 4.21 (1H, dd, J=9.3, 6.3 Hz), 3.50 (1H, dd, J=11.2, 9.3 Hz), 3.39 (3H, s), 1.70 (3H, s), 1.69 (3H, s).

MS (ESI) 561 [M+H]+

### Example 60

### Synthesis of N-(2-{[(3R)-5-(3,5-dichlorophenyl)-7,8-difluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 3, using the compound obtained in Reference Example 33.

The analytical data of the title compound are shown below.

1H-NMR (CDCl3) δ: 7.82-7.77 (2H, m), 7.20-7.04 (5H, m), 6.89 (1H, t, J=1.7 Hz), 6.68 (1H, s), 6.56 (2H, d, J=1.7 Hz), 4.72 (1H, dt, J=11.5, 5.9 Hz), 4.20 (1H, dd, J=9.5, 5.9 Hz), 3.52 (1H, dd, J=11.5, 9.5 Hz), 3.37 (3H, s), 1.70 (3H, s), 1.68 (3H, s).

MS (ESI) 579 [M+H]+

### Example 61

### Synthesis of N-[2-({(3R)-1-ethyl-8-fluoro-2-oxo-5-[4-(trifluoromethoxy)phenyl]-2,3,4,5-tetrahydro-1H-1,5-benzodiazepin-3-yl}amino)-1,1-dimethyl-2-oxoethyl]-4-fluorobenzamide

The title compound was obtained as a colorless oily substance by the method as in Example 1, using iodoethane in Reference Example 1 (Step 4).

The analytical data of the title compound are shown below.

¹H-NMR (CDCl₃) δ: 1.70 (6H, s), 3.46-3.58 (2H, m), 4.22 (1H, dd, J=9.4, 6.2 Hz), 4.33-4.40 (1H, m), 4.70 (1H, dt, J=12.5, 6.2 Hz), 6.68 (2H, d, J=8 Hz), 6.79 (1H, s), 6.97-7.21 (8H, m), 7.78-7.82 (2H, m).

ESI-MS (m/e): 591 [M+H]+

### Example 62

### Synthesis of 4-fluoro-N-[2-({(3R)-8-fluoro-1-isopropyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide

The title compound was obtained as a colorless oily substance by the method as in Example 1, using 2-bromopropane in Reference Example 1 (Step 4).

The analytical data of the title compound are shown below.

¹H-NMR (CDCl₃) δ: 1.20 (3H, d, J=6.6 Hz), 1.49 (3H, d, J=6.6 Hz), 1.74 (6H, s), 3.53 (1H, dd, J=11.7, 9.4 Hz), 4.17 (12H, dd, J=9.4, 6.2 Hz), 4.63 (1.1H, dt, J=11.7, 6.2 Hz), 4.79-4.86 (1H, m), 6.73 (2H, d, J=8 Hz), 6.84 (1H, s), 7.04-7.26 (8H, m), 7.81-7.85 (2H, m).

ESI-MS (m/e): 605 [M+H]+

### Example 63

### Synthesis of N-[1-({[(3R)-5-benzyl-1-methyl-2-oxo-2,3,4,5-tetrahydro-1H-1,5-benzodiazepin-3-yl]amino}carbonyl)cyclopropyl]benzamide

Potassium carbonate and benzyl bromide were added to the compound obtained in Reference Example 13 in 1 ml of DMF at room temperature. The mixture was stirred at room temperature for 10 hours, followed by adding a saturated aqueous ammonium chloride solution and diluting the mixture with ethyl acetate. The organic layer was washed with a saturated saline solution and dried over anhydrous magnesium sulfate. The residue obtained by removing the solvent under reduced pressure was purifies by column chromatography to yield the title compound as a white solid. The analytical data of the title compound are shown below.

1H-NMR (CDCl3) δ: 1.04-1.15 (2H, m), 1.55-1.64 (2H, m), 3.15 (1H, dd, J=11.2, 9.2 Hz), 3.37 (3H, s), 3.48 (1H, d d, J=9.6, 7.2 Hz), 4.02 (1H, d, J=13.6 Hz), 4.37 (1H, d, J=13.6 Hz), 4.46 (1H, ddd, J=11.2, 7.2, 6.8 Hz), 6.78 (1H, s), 7.05-7.28 (9H, m), 7.37-7.53 (4H, m), 7.78 (2H, d, J=7.2 Hz).

ESI-MS (m/e): 469 [M+H]+

### Example 64

### Synthesis of N-{1-[({(3R)-5-[3,5-bis(trifluoromethyl)benzyl]-1-methyl-2-oxo-2,3,4,5-tetrahydro-1H-1,5-benzodiazepin-3-yl}amino)carbonyl]cyclopropyl}benzamide

The title compound was obtained as a white solid by the method as in Example 63, using 1-(bromomethyl)-3,5-bis(trifluoromethyl)benzene.

The analytical data of the title compound are shown below.

1H-NMR (CDCl3) δ: 1.05-1.35 (3H, m), 1.57-1.60 (1H, m), 3.18-3.23 (1H, m), 3.40 (3H, s), 3.70-3.74 (1H, m), 4.16 (1H, d, J=16 Hz), 4.54 (1H, d, J=16 Hz), 4.60-4.65 (1H, m), 6.66 (1H, brs), 7.00-7.18 (4H, m), 7.41-7.55 (4H, m), 7.66-7.68 (3H, m), 7.79-7.81 (2H, m)

ESI-MS (m/e): 605 [M+H]+

### Example 65

### Synthesis of N-[1-({[(3R)-5-(3,5-dichlorobenzyl)-1-methyl-2-oxo-2,3,4,5-tetrahydro-1H-1,5-benzodiazepin-3-yl]amino}carbonyl)cyclopropyl]benzamide

The title compound was obtained as a white solid by the method as in Example 63, using 1-(bromomethyl)-3,5-dichlorobenzene.

The analytical data of the title compound are shown below.

1H-NMR (CDCl3) δ: 1.07-1.19 (2H, m), 1.55-1.69 (2H, m), 3.18 (1H, dd, J=11.2, 9.2 Hz), 3.37 (3H, s), 3.58 (1H, d d, J=9.2, 7.2 Hz), 3.95 (1H, d, J=14.4 Hz), 4.34 (1H, d, J=14.8 Hz), 4.64 (1H, ddd, J=11.2, 6.8, 6.8 Hz), 6.89 (1H, s), 7.03-7.16 (7H, m), 7.44-7.53 (4H, m), 7.78 (2H, d, J=7.2 Hz).

ESI-MS (m/e): 537 [M+H]+

### Example 66

### Synthesis of N-[1-({[(3R)-5-(4-fluorobenzyl)-1-methyl-2-oxo-2,3,4,5-tetrahydro-1H-1,5-benzodiazepin-3-yl]amino}carbonyl)cyclopropyl]benzamide

The title compound was obtained as a white solid by the method as in Example 63, using 1-(bromomethyl)-4-fluorobenzene.

The analytical data of the title compound are shown below.

1H-NMR (CDCl3) δ: 1.04-1.16 (2H, m), 1.54-1.65 (2H, m), 3.11 (1H, dd, J=11.6, 9.6 Hz), 3.35 (3H, s), 3.46 (1H, d d, J=9.6, 6.8 Hz), 3.98 (1H, d, J=14 Hz), 4.32 (1H, d, J=13.6 Hz), 4.62 (1H, ddd, J=11.2, 7.2, 7.2 Hz), 6.81 (1H, s), 6.94 (2H, dd, J=8.8, 8.8 Hz), 6.96-7.21 (6H, m), 7.41-7.52 (4H, m), 7.80 (2H, d, J=6.8 Hz).

ESI-MS (m/e): 487 [M+H]+

### Example 67

### Synthesis of N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide

The title compound was obtained as a white solid by the method as in Example 63, using 1-(bromomethyl)-4-(trifluoromethoxy)benzene.

The analytical data of the title compound are shown below.

1H-NMR (CDCl3) δ: 1.08-1.14 (2H, m), 1.22-1.25 (1H, m), 1.58-1.63 (1H, m), 3.09-3.14 (1H, m), 3.39 (3H, s), 3.48-3.52 (1H, m), 4.02 (1H, d, J=12 Hz), 4.36 (1H, d, J=12 Hz), 4.61-4.67 (1H, m), 6.64 (1H, brs), 7.09-7.20 (5H, m), 7.25-7.28 (3H, m), 7.35-7.36 (1H, m), 7.43-7.47 (2H, m), 7.52-7.54 (1H, m), 7.76-7.78 (3H, m)

ESI-MS (m/e): 553 [M+H]+

### Example 68

### Synthesis of 4-fluoro-N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide

The title compound was obtained as a white solid by the method as in Example 63, using the compound obtained in Reference Example 14 and 1-(bromomethyl)-4-(trifluoromethoxy)benzene.

The analytical data of the title compound are shown below.

(2H, m), 3.08 (1H, t, J=10.4 Hz), 3.32 (3H, s), 3.43 (1H, dd, J=9.4, 7 Hz), 3.97 (1H, d, J=14.1 Hz), 4.32 (1H, d, J=14.1 Hz), 4.59 (1H, dt, J=12.9, 5.7 Hz), 6.87 (1H, s), 7.03-7.23 (1H, m), 7.38 (1H, d, J=7 Hz), 7.75-7.77 (2H, m).

ESI-MS (m/e): 571 [M+H]+

### Example 69

### Synthesis of N-{1-[({(3R)-8-fluoro-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide

The title compound was obtained as a white solid by the method as in Example 67, using the compound obtained in Reference Example 5.

The analytical data of the title compound are shown below.

¹H-NMR (CDCl₃) δ: 1.07-1.17 (2H, m), 1.56-1.70 (2H, m), 3.19-3.14 (1H, m), 3.37 (3H, s), 3.48 (1H, dd, J=9.2, 7.2 Hz), 4.01 (1H, d, J=14.1 Hz), 4.29 (1H, d, J=14.1 Hz), 4.61 (1H, dt, J=13.0, 5.6 Hz), 6.71 (1H, s), 6.89-6.93 (2H, m), 7.03-7.06 (1H, m), 7.11 (2H, d, J=8 Hz), 7.23 (2H, d, J=8 Hz), 7.36 (1H, d, J=7.4 Hz), 7.43-7.47 (2H, m), 7.52-7.54 (1H, m), 7.78 (2H, d, J=7 Hz).

ESI-MS (m/e): 571 [M+H]+

### Example 70

### Synthesis of N-{1-[({(3R)-5-[3,5-bis(trifluoromethyl)benzyl]-8-fluoro-1-methyl-2-oxo-2,3,4,5-tetrahydro-1H-1,5-benzodiazepin-3-yl}amino)carbonyl]cyclopropyl}benzamide

The title compound was obtained as a white solid by the method as in Example 64, using the compound obtained in Reference Example 5.

The analytical data of the title compound are shown below.

1H-NMR (CDCl3) δ: 1.09-1.12 (1H, m), 1.15-1.18 (1H, m), 1.57-1.62 (1H, m), 1.65-1.69 (1H, m), 318-3.23 (1H, m), 3.38 (3H, s), 3.70-3.74 (1H, m), 4.15 (1H, d, J=15.1 Hz), 4.47 (1H, d, J=15.1 Hz), 4.60-4.67 (1H, m), 6.66 (1H, s), 6.85-6.92 (2H, m), 6.97-7.01 (1H, m), 7.40 (1H, d, J=6.8 Hz), 7.47 (2H, t, J=7.3 Hz), 7.53-7.57 (1H, m), 7.63 (2H, s), 7.69 (1H, s), 7.78-7.80 (2H, m).

ESI-MS (m/e): 623 [M+H]+

### Example 71

### Synthesis of N-[1-({[(3R)-5-benzyl8-fluoro-1-methyl-2-oxo-2,3,4,5-tetrahydro-1H-1,5-benzodiazepin-3-yl]amino}carbonyl)cyclopropyl]benzamide

The title compound was obtained as a white solid by the method as in Example 63, using the compound obtained in Reference Example 5.

The analytical data of the title compound are shown below.

1H-NMR (DMSO-D6) δ: 0.90-0.98 (2H, m), 1.18-1.25 (2H, m), 3.08-3.21 (2H, m), 3.22 (3H, s), 4.02 (1H, d, J=14.1 Hz), 4.27 (1H, d, J=14.1 Hz), 4.31-4.37 (1H, m), 7.00-7.05 (1H, m), 7.11-7.23 (6H, m), 7.29 (1H, dd, J=9.8, 3.1 Hz), 7.41 (2H, t, J=7.4 Hz), 7.49 (1H, t, J=7.4 Hz), 7.73 (1H, d, J=7.8 Hz), 7.80-7.82 (2H, m), 8.89 (1H, s).

ESI-MS (m/e): 487 [M+H]+

### Example 72

### Synthesis of N-[1-({[(3R)-5-(4-chlorobenzyl)-8-fluoro-1-methyl-2-oxo-2,3,4,5-tetrahydro-1H-1,5-benzodiazepin-3-yl]amino}carbonyl)cyclopropyl]benzamide

The title compound was obtained as a white solid by the method as in Example 63, using the compound obtained in Reference Example 5 and 1-(bromomethyl)-4-chlorobenzene.

The analytical data of the title compound are shown below.

1H-NMR (DMSO-D6) δ: 0.88-0.98 (2H, m), 1.19-1.25 (2H, m), 3.06-3.28 (2H, m), 3.28 (3H, s), 4.02 (1H, d, J=14.1 Hz), 4.26 (1H, d, J=14.1 Hz), 4.30-4.37 (1H, m), 7.02 (1H, td, J=8.4, 3.1 Hz), 7.17-7.22 (3H, m), 7.26-7.31 (3H, m), 7.41 (2H, t, J=7.3 Hz), 7.49 (1H, t, J=7.3 Hz), 7.74 (1H, d, J=7.2 Hz), 7.80-7.83 (2H, m), 8.89 (1H, s).

ESI-MS (m/e): 521 [M+H]+

### Example 73

### Synthesis of N-[1-({[(3R)-5-(4-cyanobenzyl)-8-fluoro-1-methyl-2-oxo-2,3,4,5-tetrahydro-1H-1,5-benzodiazepin-3-yl]amino}carbonyl)cyclopropyl]benzamide

The title compound was obtained as a white solid by the method as in Example 63, using the compound obtained in Reference Example 5 and 1-(bromomethyl)-4-cyanobenzene.

The analytical data of the title compound are shown below.

1H-NMR (DMSO-D6) δ: 0.88-0.99 (2H, m), 1.19-1.25 (2H, m), 3.13-3.20 (2H, m), 3.23 (3H, s), 4.13 (1H, d, J=14.9 Hz), 4.31-4.38 (1H, m), 4.40 (1H, d, J=14.9 Hz), 7.02 (1H, td, J=8.5, 2.9 Hz), 7.19-7.23 (1H, m), 7.27-7.36 (3H, m), 7.42 (2H, t, J=7.3 Hz), 7.49 (1H, t, J=7.3 Hz), 7.69 (2H, d, J=8.6 Hz), 7.74 (1H, d, J=7.8 Hz), 7.80-7.82 (2H, m), 8.89 (1H, s).

ESI-MS (m/e): 512 [M+H]+

### Example 74

### Synthesis of N-{1-[({(3R)-5-[4-(difluoromethoxy)benzyl]-8-fluoro-1-methyl-2-oxo-2,3,4,5-tetrahydro-1H-1,5-benzodiazepin-3-yl}amino)carbonyl]cyclopropyl}benzamide

The title compound was obtained as a white solid by the method as in Example 63, using the compound obtained in Reference Example 5 and 1-(bromomethyl)-4-(difluoromethoxy)benzene.

The analytical data of the title compound are shown below.

1H-NMR (DMSO-D6) δ: 0.91-0.98 (2H, m), 1.18-1.25 (2H, m), 3.08-3.20 (2H, m), 3.22 (3H, s), 4.01 (1H, d, J=14.1 Hz), 4.26 (1H, d, J=14.1 Hz), 4.30-4.35 (1H, m), 6.94-7.12 (4H, m), 7.19-7.23 (3H, m), 7.28-7.31 (1H, m), 7.41 (2H, t, J=7.3 Hz), 7.49 (1H, t, J=7.3 Hz), 7.74 (1H, d, J=7.8 Hz), 7.80-7.82 (2H, m), 8.89 (1H, s).

ESI-MS (m/e): 553 [M+H]+

### Example 75

### Synthesis of N-[1-({[(3R)-5-(3,5-difluorobenzyl)-8-fluoro-1-methyl-2-oxo-2,3,4,5-tetrahydro-1H-1,5-benzodiazepin-3-yl]amino}carbonyl)cyclopropyl]benzamide

The title compound was obtained as a white solid by the method as in Example 63, using the compound obtained in Reference Example 5 and 1-(bromomethyl)-3,5-difluorobenzene.

The analytical data of the title compound are shown below.

1H-NMR (DMSO-D6) δ: 0.88-1.00 (2H, m), 1.18-1.26 (2H, m), 3.15-3.28 (2H, m), 3.28 (3H, m), 4.08 (1H, d, J=14.5 Hz), 4.30-4.37 (2H, m), 6.85-6.87 (1H, m), 7.00-7.10 (2H, m), 7.17-7.30 (3H, m), 7.42 (2H, d, J=7.3 Hz), 7.49 (1H, t, J=7.3 Hz), 7.76-7.80 (1H, m), 7.81-7.83 (2H, m), 8.90 (1H, s).

ESI-MS (m/e): 523 [M+H]+

### Example 76

### Synthesis of N-[1-({[(3R)-8-fluoro-5-(3-fluorobenzyl)-1-methyl-2-oxo-2,3,4,5-tetrahydro-1H-1,5-benzodiazepin-3-yl]amino}carbonyl)cyclopropyl]benzamide

The title compound was obtained as a white solid by the method as in Example 63, using the compound obtained in Reference Example 5 and 1-(bromomethyl)-3-fluorobenzene.

The analytical data of the title compound are shown below.

1H-NMR (DMSO-D6) δ: 0.90-0.98 (2H, m), 1.19-1.22 (2H, m), 3.15-3.23 (5H, m), 4.07 (1H, d, J=14.5 Hz), 4.30-4.35 (2H, m), 7.02-7.09 (3H, m), 7.16-7.31 (4H, m), 7.41 (2H, t, J=7.4 Hz), 7.49 (1H, t, J=7.4 Hz), 7.77 (1H, d, J=4 Hz), 7.81 (2H, d, J=7.2 Hz), 8.90 (1H, s).

ESI-MS (m/e): 505 [M+H]+

### Example 77

### Synthesis of N-[1-({[(3R)-8-fluoro-5-(4-fluorobenzyl)-1-methyl-2-oxo-2,3,4,5-tetrahydro-1H-1,5-benzodiazepin-3-yl]amino}carbonyl)cyclopropyl]benzamide

The title compound was obtained as a white solid by the method as in Example 66, using the compound obtained in Reference Example 5.

The analytical data of the title compound are shown below.

1H-NMR (DMSO-D6) δ: 0.88-0.98 (2H, m), 1.18-1.22 (2H, m), 3.07-3.20 (2H, m), 3.21 (3H, s), 4.00 (1H, d, J=14.1 Hz), 4.25 (1H, d, J=14.1 Hz), 4.30-4.38 (1H, m), 7.00-7.06 (3H, m), 7.17-7.22 (3H, m), 7.29 (1H, dd, J=9.8, 3.1 Hz), 7.41 (2H, t, J=7.1 Hz), 7.49 (1H, t, J=7.1 Hz), 7.73 (1H, d, J=7.8 Hz), 7.81 (2H, d, J=7.1 Hz), 8.89 (1H, s).

ESI-MS (m/e): 505 [M+H]+

### Example 78

### Synthesis of N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]benzamide

The title compound was obtained as a colorless oily substance by the method as in Example 67, using the compound obtained in Reference Example 4.

The analytical data of the title compound are shown below.

¹H-NMR (CDCl₃) δ: 1.60 (3H, s), 1.61 (3H, s), 3.07 (1H, dd, J=11.9, 9.6 Hz), 3.37 (3H, s), 3.51 (1H, dd, J=9.6, 6.3 Hz), 4.01 (1H, d, J=13.9 Hz), 4.28 (1H, d, J=13.9 Hz), 4.59 (1H, dt, J=11.9, 6.3 Hz), 6.79 (1H, s), 6.86-6.92 (2H, m), 6.99-7.05 (2H, m), 7.09 (2H, d, J=8.6 Hz), 7.22 (2H, d, J=8.6 Hz), 7.35-7.39 (2H, m), 7.44-7.47 (1H, m), 7.68-7.70 (2H, m).

ESI-MS (m/e): 573 [M+H]+

### Example 79

### Synthesis of 4-fluoro-N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]benzamide

The title compound was obtained as a white solid by the method as in Example 67, using the compound obtained in Reference Example 3.

The analytical data of the title compound are shown below.

¹H-NMR (CDCl₃) δ: 1.64 (3H, s), 1.65 (3H, s), 3.11 (1H, dd, J=11.9, 9.6 Hz), 3.42 (3H, s), 3.54 (1H, dd, J=9.6, 6.3 Hz), 4.05 (1H, d, J=14.1 Hz), 4.32 (1H, d, J=14.1 Hz), 4.61 (1H, dt, J=11.9, 6.3 Hz), 6.82 (1H, s), 6.91-6.93 (2H, m), 7.01 (1H, d, J=6.6 Hz), 7.05-7.14 (5H, m), 7.25-7.27 (2H, m), 7.74 (2H, dd, J=8.8, 5.3 Hz).

ESI-MS (m/e): 591 [M+H]+

### Example 80

### Synthesis of N-(2-{[(3R)-5-(3,5-dichlorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a pale yellow solid by the method as in Example 65, using the compound obtained in Reference Example 3.

The analytical data of the title compound are shown below.

1H-NMR (DMSO-D6) δ: 1.39 (6H, s), 3.11-3.26 (2H, m), 3.28 (3H, s), 4.11 (1H, d, J=14.7 Hz), 4.32-4.44 (1H, m), 4.37 (1H, d, J=14.7 Hz), 7.10 (1H, td, J=8.4, 2.9 Hz), 7.21-7.31 (5H, m), 7.39 (1H, dd, J=9.8, 2.9 Hz), 7.42-7.46 (1H, m), 7.68 (1H, d, J=8.4 Hz), 7.84-7.91 (2H, m), 8.27 (1H, s).

ESI-MS (m/e): 576 [M+H]+

### Example 81

### Synthesis of 4-fluoro-N-[2-({(3R)-8-fluoro-1-methyl-2-oxo-5-[4-(trifluoromethyl)benzyl]-2,3,4,5-tetrahydro-1H-1,5-benzodiazepin-3-yl}amino)-1,1-dimethyl-2-oxoethyl]benzamide

The title compound was obtained as a white solid by the method as in Example 63, using the compound obtained in Reference Example 3 and 1-(bromomethyl)-4-(trifluoromethyl)benzene.

The analytical data of the title compound are shown below.

1H-NMR (DMSO-D6) δ: 1.32 (3H, s), 1.34 (3H, s), 3.04-3.17 (2H, m), 3.24 (3H, s), 4.13 (1H, d, J=14.5 Hz), 4.30-4.37 (1H, m), 4.39 (1H, d, J=14.5 Hz), 7.04 (1H, td, J=8.5, 2.9 Hz), 7.19-7.24 (3H, m), 7.33 (1H, dd, J=9.6, 2.9 Hz), 7.38 (2H, d, J=8.2 Hz), 7.60-7.61 (3H, m), 7.79-7.82 (2H, m), 8.19 (1H, s).

ESI-MS (m/e): 575 [M+H]+

### Example 82

### Synthesis of 4-fluoro-N-(2-{[(3R)-8-fluoro-1-methyl-2-oxo-5-(2-phenylethyl)-2,3,4,5-tetrahydro-1H-1,5-benzodiazepin-3-yl]amino}-1,1-dimethyl-2-oxoethyl)benzamide

The title compound was obtained as a white solid by the method as in Example 63, using the compound obtained in Reference Example 3 and 2-(bromomethyl)benzene.

The analytical data of the title compound are shown below.

1H-NMR (DMSO-D6) δ: 1.40 (6H, s), 2.56-2.72 (2H, m), 3.01-3.14 (5H, m), 3.28-3.41 (2H, m), 4.23-4.34 (1H, m), 7.00-7.35 (10H, m), 7.63 (1H, d, J=7.8 Hz), 7.86-7.94 (2H, m), 8.31 (1H, s).

ESI-MS (m/e): 621 [M+H]+

### Example 83

### Synthesis of N-[2-({(3R)-5-[3,5-bis(trifluoromethyl)benzyl]-8-fluoro-1-methyl-2-oxo-2,3,4,5-tetrahydro-1H-1,5-benzodiazepin-3-yl}amino)-1,1-dimethyl-2-oxoethyl]-4-fluorobenzamide

The title compound was obtained as a white solid by the method as in Example 64, using the compound obtained in Reference Example 3.

The analytical data of the title compound are shown below.

1H-NMR (DMSO-D6) δ: 1.33 (3H, s), 1.36 (3H, s), 3.21 (3H, s), 3.21-3.24 (2H, m), 4.24 (1H, d, J=15.2 Hz), 4.30-4.34 (1H, m), 4.58 (1H, d, J=15.2 Hz), 7.00-7.05 (1H, m), 7.20-7.27 (3H, m), 7.33 (1H, dd, J=9.8, 2.7 Hz), 7.61 (1H, d, J=7.8 Hz), 7.80-7.84 (4H, m), 7.88 (1H, s), 8.23 (1H, s).

ESI-MS (m/e): 643 [M+H]+

### Example 84

### Synthesis of 4-fluoro-N-[2-({(3R)-8-fluoro-5-[4-fluoro-3-(trifluoromethyl)benzyl]-1-methyl-2-oxo-2,3,4,5-tetrahydro-1H-1,5-benzodiazepin-3-yl}amino)-1,1-dimethyl-2-oxoethyl]benzamide

The title compound was obtained as a white solid by the method as in Example 63, using the compound obtained in Reference Example 3 and 4-(bromomethyl)-1-fluoro-2-(trifluoromethyl)benzene.

The analytical data of the title compound are shown below.

1H-NMR (DMSO-D6) δ: 1.38 (6H, s), 3.07-3.36 (5H, m), 4.15 (1H, d, J=14.6 Hz), 4.30-4.47 (2H, m), 7.08 (1H, td, J=8.8, 2.9 Hz), 7.20-7.31 (3H, m), 7.31-7.47 (2H, m), 7.52-7.68 (3H, m), 7.81-7.91 (2H, m), 8.26 (1H, s).

ESI-MS (m/e): 593 [M+H]+

### Example 85

### Synthesis of N-(2-{[(3R)-5-(2-chlorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 63, using the compound obtained in Reference Example 3 and 1-(bromomethyl)-2-chlorobenzene.

The analytical data of the title compound are shown below.

1H-NMR (DMSO-D6) δ: 1.38 (6H, s), 3.17-3.27 (5H, m), 4.12 (1H, d, J=14.1 Hz), 4.33-4.41 (2H, m), 7.10 (1H, td, J=8.4, 3.1 Hz), 7.22-7.41 (8H, m), 7.63 (1H, d, J=8.4 Hz), 7.86 (2H, dt, J=9.4, 2.9 Hz), 8.22 (1H, s).

ESI-MS (m/e): 542 [M+H]+

### Example 86

### Synthesis of 4-fluoro-N-[2-({(3R)-8-fluoro-1-methyl-2-oxo-5-[2-(trifluoromethyl)benzyl]-2,3,4,5-tetrahydro-1H-1,5-benzodiazepin-3-yl}amino)-1,1-dimethyl-2-oxoethyl]benzamide

The title compound was obtained as a white solid by the method as in Example 63, using the compound obtained in Reference Example 3 and 1-(bromomethyl)-2-(trifluoromethyl)benzene.

The analytical data of the title compound are shown below.

1H-NMR (DMSO-D6) δ: 1.37 (6H, s), 3.05-3.18 (2H, m), 3.25 (3H, s), 4.20 (1H, d, J=14.3 Hz), 4.31-4.42 (1H, m), 4.52 (1H, d, J=14.3 Hz), 7.10 (1H, td, J=8.2, 2.9 Hz), 7.22-7.31 (3H, m), 7.37 (1H, dd, J=9.8, 2.9 Hz), 7.41-7.53 (2 H, m), 7.53-7.72 (3H, m), 7.79-7.88 (2H, m), 8.21 (1H, s).

ESI-MS (m/e): 575 [M+H]+

### Example 87

### Synthesis of 4-fluoro-N-(2-{[(3R)-8-fluoro-5-(2-fluorobenzyl)-1-methyl-2-oxo-2,3,4,5-tetrahydro-1H-1,5-benzodiazepin-3-yl]amino}-1,1-dimethyl-2-oxoethyl)benzamide

The title compound was obtained as a white solid by the method as in Example 63, using the compound obtained in Reference Example 3 and 1-(bromomethyl)-2-fluorobenzene.

The analytical data of the title compound are shown below.

1H-NMR (DMSO-D6) δ: 1.38 (6H, s), 3.07-3.26 (5H, m), 4.12 (1H, d, J=14.1 Hz), 4.33-4.39 (2H, m), 7.05-7.17 (3H, m), 7.20-7.38 (6H, m), 7.64 (1H, d, J=8.2 Hz), 7.81-7.89 (2H, m), 8.23 (1H, s).

ESI-MS (m/e): 525 [M+H]+

### Example 88

### Synthesis of N-[2-({(3R)-5-[2-(difluoromethoxy)benzyl]-8-fluoro-1-methyl-2-oxo-2,3,4,5-tetrahydro-1H-1,5-benzodiazepin-3-yl}amino)-1,1-dimethyl-2-oxoethyl]-4-fluorobenzamide

The title compound was obtained as a white solid by the method as in Example 63, using the compound obtained in Reference Example 3 and 1-(bromomethyl)-2-(difluoromethoxy)benzene.

The analytical data of the title compound are shown below.

1H-NMR (DMSO-D6) δ: 1.38 (6H, s), 3.07-3.26 (5H, m), 4.07 (1H, d, J=14.5 Hz), 4.29-4.41 (2H, m), 6.80-7.39 (10H, m), 7.62 (1H, d, J=8.2 Hz), 7.82-7.89 (2H, m), 8.22 (1H, s).

ESI-MS (m/e): 573 [M+H]+

### Example 89

### Synthesis of N-(2-{[(3R)-5-(4-chloro-3-fluorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained as a white solid by the method as in Example 63, using the compound obtained in Reference Example 3 and 4-(bromomethyl)-1-chloro-2-fluorobenzene.

The analytical data of the title compound are shown below.

1H-NMR (DMSO-D6) δ: 1.38 (6H, s), 3.09 (1H, dd, J=9.4, 7.4 Hz), 3.18 (1H, dd, J=11.3, 9.4 Hz), 3.27 (3H, s), 4.09 (1H, d, J=14.5 Hz), 4.29-4.42 (2H, m), 7.04-7.14 (2H, m), 7.17-7.31 (4H, m), 7.37 (1H, dd, J=10.0, 2.9 Hz), 7.49 (1H, t, J=7.9 Hz), 7.62 (1H, d, J=7.9 Hz), 7.80-7.90 (2H, m), 8.24 (1H, s).

ESI-MS (m/e): 559 [M+H]+

### Example 90

### Synthesis of N-{1-[({(3R)-5-[3,5-bis(trifluoromethyl)benzyl]-8-chloro-1-methyl-2-oxo-2,3,4,5-tetrahydro-1H-1,5-benzodiazepin-3-yl}amino)carbonyl]cyclopropyl}benzamide

The title compound was obtained as a white solid by the method as in Example 64, using the compound obtained in Reference Example 17.

The analytical data of the title compound are shown below.

1H-NMR (DMSO-D6) δ: 0.89-1.03 (2H, m), 1.19-1.28 (2H, m), 3.20 (3H, s), 3.25-3.37 (2H, m), 4.23 (1H, d, J=15.2 Hz), 4.34-4.41 (1H, m), 4.60 (1H, d, J=15.2 Hz), 7.19-7.26 (2H, m), 7.41 (2H, t, J=7.2 Hz), 7.48-7.51 (2H, m), 7.77-7.87 (6H, m), 8.92 (1H, s).

ESI-MS (m/e): 639 [M+H]+

### Example 91

### Synthesis of N-{1-[({(3R)-1-[3,5-bis(trifluoromethyl)benzyl]-4-oxo-2,3,4,5-tetrahydro-1H-1,5-benzodiazepin-3-yl}amino)carbonyl]cyclopropyl}benzamide

The title compound was obtained by the method as in Example 64, using the compound obtained in Reference Example 15.

The analytical data of the title compound are shown below.

1H-NMR (DMSO-D6) δ: 0.89-1.02 (2H, m), 1.20-1.32 (2H, m), 3.27-3.32 (2H, m), 4.24-4.28 (1H, m), 4.31-4.40 (1H, m), 4.59-4.63 (1H, m), 6.93-7.15 (4H, m), 7.38-7.48 (3H, m), 7.70-7.72 (1H, m), 7.79-7.81 (2H, m), 7.87 (3 H, brs), 8.91 (1H, s), 9.97 (1H, s).

ESI-MS (m/e): 591 [M+H]+

### Example 92

### Synthesis of tert-butyl {1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}carbamate

The title compound was obtained as a white solid by the method as in Example 67, using the compound obtained in Reference Example 24.

The analytical data of the title compound are shown below.

1H-NMR (DMSO-D6) δ: 0.72-0.89 (2H, m), 1.02-1.28 (2H, m), 1.30 (9H, s), 2.95-3.08 (1H, m), 3.15-3.20 (1H, m), 3.30 (3H, s), 4.08-4.12 (1H, m), 4.26-4.40 (2H, m), 7.10-7.60 (1H, m)

ESI-MS (m/e): 549 [M+H]+

### Example 93

### Synthesis of 4-chloro-N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide

The title compound was obtained as a white solid by the method as in Reference Example 3, using the compound obtained in Reference Example 25 and 4-chlorobenzoic acid.

The analytical data of the title compound are shown below.

1H-NMR (CDCl3) δ: 1.00-1.10 (2H, m), 1.48-1.60 (2H, m), 3.09 (1H, dd, J=11.1, 9.6 Hz), 3.29 (3H, s), 3.43 (1H, d d, J=9.4, 7 Hz), 3.97 (1H, d, J=14.1 Hz), 4.32 (1H, d, J=14.1 Hz), 4.59 (1H, dt, J=13.0, 5.6 Hz), 6.96 (1H, s), 7.04-7.21 (8H, m), 7.34 (2H, d, J=8.6 Hz), 7.43 (1H, d, J=7 Hz), 7.68 (2H, d, J=8.6 Hz).

ESI-MS (m/e): 587 [M+H]+

### Example 94

### Synthesis of N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}pyridine-2-carboxyamide

The title compound was obtained as a white solid by the method as in Reference Example 3, using the compound obtained in Reference Example 25 and 2-pyridinecarboxylic acid.

The analytical data of the title compound are shown below.

1H-NMR (CDCl3) δ: 1.04-1.14 (2H, m), 1.52-1.62 (2H, m), 3.08 (1H, t, J=10.4 Hz), 3.34 (3H, s), 3.46 (1H, dd, J=9.2, 7.2 Hz), 3.97 (1H, d, J=13.7 Hz), 4.32 (1H, d, J=14.1 Hz), 4.60 (1H, dt, J=12.9, 5.7 Hz), 7.08-7.17 (8H, m), 7.32 (1H, d, J=6.6 Hz), 7.42 (1H, dd, J=7.4, 4.7 Hz), 7.82 (1H, dd, J=8.4, 6.8 Hz), 8.14 (1H, d, J=7.8 Hz), 8.49-8.51 (2H, m)

ESI-MS (m/e): 554 [M+H]+

### Example 95

### 1-ethylpropyl {1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}carbamate

Iodomethane was added to a solution of 1-ethylpropyl 1H-imidazol-1-carboxylic acid in acetonitrile, and the mixture was stirred at room temperature for 48 hours. The solvent was distilled off, followed by adding a compound obtained in Reference Example 25 and chloroform to residual acetonitrile solution and stirring the mixture at room temperature overnight. The solvent was distilled off, followed by adding water and to the residue and extracting the mixture with ethyl acetate. The organic layer was washed with a saturated saline solution and thereafter dried over anhydrous magnesium sulfate. After filtration, the solvent was distilled off, and the residue was purified by silica gel column chromatography (hexane-ethyl acetate) to yield the title compound as a white solid.

The analytical data of the title compound are shown below.

1H-NMR (CDCl3) δ: 0.85-1.55 (14H, m), 3.02 (1H, dd, J=11.1, 9.6 Hz), 3.39 (3H, s), 3.97 (1H, d, J=13.7 Hz), 4.33 (1H, d, J=14.1 Hz), 4.55-4.61 (2H, m), 7.08-7.15 (5H, m), 7.25-7.32 (6H, m).

ESI-MS (m/e): 563 [M+H]+

### Example 96

### Synthesis of 5-fluoro-N-[2-({(3R)-8-fluoro-1-methyl-2-oxo-5-trifluoromethoxy)benzyl-2,3,4,5-tetrahydro-1H-1,5-benzodiazepin-3-yl}amino)-1,1-dimethyl-2-oxoethyl]pyridine-2-carboxyamide

The title compound was obtained as a white solid by the method as in Example 44, using a compound obtained in Reference Example 26.

The analytical data of the title compound are shown below.

1H-NMR (CDCl3) δ: 1.62 (6H, s), 3.09 (1H, dd, J=11.1, 9.6 Hz), 3.39 (3H, s), 3.55 (1H, dd, J=9.4, 7 Hz), 4.04 (1H, d, J=14.1 Hz), 4.31 (1H, d, J=14.1 Hz), 4.61-4.64 (1H, m), 6.93 (2H, t, J=5.1 Hz), 7.10 (4H, ddd, J=33.5, 13.2, 6.5 Hz), 7.25 (2H, d, J=8.6 Hz), 7.51 (1H, td, J=8.4, 2.7 Hz), 8.13 (1H, dd, J=8.8, 4.5 Hz), 8.30 (1H, s), 8.37 (1H, d, J=2.7 Hz).

ESI-MS (m/e): 563.4 [M+H]+

### Example 97

### Synthesis of N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]-1,3-thiazol-2-carboxyamide

The title compound was obtained as a white solid by the method as in Example 45, using the compound obtained in Reference Example 26.

The analytical data of the title compound are shown below.

1H-NMR (CDCl3) δ: 1.63 (6H, s), 3.10 (1H, dd, J=10.9, 9.8 Hz), 3.40 (3H, s), 3.55 (1H, dd, J=9.4, 7 Hz), 4.04 (1H, d, J=14.1 Hz), 4.31 (1H, d, J=14.1 Hz), 4.63 (1H, dt, J=13.0, 5.6 Hz), 6.92 (2H, dq, J=12.5, 3.4 Hz), 7.09 (4H, ddd, J=19.3, 9.9, 6.5 Hz), 7.25 (2H, d, J=8.6 Hz), 7.56 (1H, d, J=3.1 Hz), 7.74 (1H, s), 7.84 (1H, d, J=2.7 Hz).

ESI-MS (m/e): 580.3 [M+H]+

### Example 98

### Synthesis of N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]-5-methylpyrazin-2-carboxyamide

The title compound was obtained as a white solid by the method as in Reference Example 3, using the compound obtained in Reference Example 26 and 5-methylpyrazin-2-carboxylic acid.

The analytical data of the title compound are shown below.

1H-NMR (CDCl3) δ: 1.64 (6H, s), 2.63 (3H, d, J=11.7 Hz), 3.11 (1H, dd, J=11.1, 9.6 Hz), 3.40 (3H, s), 3.56 (1H, dd, J=9.4, 7 Hz), 4.04 (1H, d, J=14.1 Hz), 4.31 (1H, d, J=14.1 Hz), 4.63 (1H, dt, J=12.9, 5.6 Hz), 6.92 (2H, ddd, J=13.0, 7.5, 2.8 Hz), 7.06 (1H, dd, J=8.6, 5.5 Hz), 7.12 (3H, t, J=4.1 Hz), 7.24 (2H, d, J=8.6 Hz), 8.23 (1H, s), 8.36 (1H, d, J=0.8 Hz), 9.19 (1H, d, J=1.2 Hz).

ESI-MS (m/e): 589.4 [M+H]+

### Example 99

### Synthesis of N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]pyridine-2-carboxyamide

The title compound was obtained as a white solid by the method as in Example 94, using the compound obtained in Reference Example 26.

The analytical data of the title compound are shown below.

1H-NMR (CDCl3) δ: 1.62 (6H, s), 3.09 (1H, dd, J=11.1, 9.6 Hz), 3.38 (3H, s), 3.56 (1H, dd, J=9.4, 7 Hz), 4.04 (1H, d, J=13.7 Hz), 4.31 (1H, d, J=13.7 Hz), 4.64 (1H, dt, J=12.9, 5.6 Hz), 6.88-6.94 (2H, m), 7.08 (3H, dt, J=21.5, 7.9 Hz), 7.22 (3H, dd, J=17.4, 7.6 Hz), 7.43 (1H, ddd, J=7.8, 4.7, 1.2 Hz), 7.83 (1H, td, J=7.7, 1.7 Hz), 8.09 (1H, d, J=7.8 Hz), 8.44 (1H, s), 8.54 (1H, dd, J=4.7, 0.8 Hz).

ESI-MS (m/e): 574.4 [M+H]+

### Example 100

### Synthesis of N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]-1,3-oxazol-2-carboxyamide

The title compound was obtained as a white solid by the method as in Reference Example 3, using the compound obtained in Reference Example 26 and 1,3-oxazol-2-carboxylic acid.

The analytical data of the title compound are shown below.

1H-NMR (CDCl3) δ: 1.62 (6H, s), 3.12 (1H, dd, J=11.1, 9.6 Hz), 3.41 (3H, s), 3.55 (1H, dd, J=9.4, 7 Hz), 4.04 (1H, d, J=14.1 Hz), 4.31 (1H, d, J=14.1 Hz), 4.61 (1H, dt, J=12.9, 5.6 Hz), 6.92 (2H, dt, J=13.8, 4.7 Hz), 7.04 (2H, dt, J=16.6, 5.7 Hz), 7.12 (2H, d, J=8.6 Hz), 7.23 (3H, t, J=11.5 Hz), 7.59 (1H, s), 7.76 (1H, s).

ESI-MS (m/e): 564.4 [M+H]+

### Example 101

### Synthesis of N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]-4-(trifluoromethyl)cyclohexanecarboxyamide

The title compound was obtained as a white solid by the method as in Reference Example 3, using the compound obtained in Reference Example 26 and 4-(trifluoromethyl)cyclohexanecarboxylic acid.

The analytical data of the title compound are shown below.

1H-NMR (CDCl3) δ: 1.49 (6H, t, J=4.9 Hz), 1.64 (8H, tt, J=29.9, 10.1 Hz), 2.00 (4H, dd, J=19.4, 16.2 Hz), 3.41 (3 H, s), 3.51 (1H, dt, J=11.3, 4.8 Hz), 4.03 (1H, d, J=14.1 Hz), 4.31 (1H, d, J=14.1 Hz), 4.57 (1H, dt, J=12.8, 5.7 Hz), 6.07 (1H, s), 6.92 (3H, dt, J=16.4, 3.6 Hz), 7.06 (1H, dd, J=9.2, 5.7 Hz), 7.13 (2H, d, J=7.8 Hz), 7.25 (1H, t, J=4.7 Hz).

ESI-MS (m/e): 647.4 [M+H]+

### Example 102

### Synthesis of N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]-1-methyl-1H-imidazol-2-carboxyamide

The title compound was obtained as a white solid by the method as in Reference Example 3, using the compound obtained in Reference Example 26 and 1-methyl-1H-imidazol-2-carboxylic acid.

The analytical data of the title compound are shown below.

1H-NMR (CDCl3) δ: 1.55 (6H, s), 3.06 (1H, t, J=10.2 Hz), 3.39 (3H, s), 3.53 (1H, dd, J=9.4, 7 Hz), 3.91 (3H, s), 4.03 (1H, d, J=13.7 Hz), 4.31 (1H, d, J=14.1 Hz), 4.63 (1H, dt, J=12.9, 5.6 Hz), 6.89-6.94 (3H, m), 6.98 (1H, d, J=1.2 Hz), 7.06 (1H, dd, J=9.4, 5.5 Hz), 7.14 (3H, dd, J=14.1, 7.4 Hz), 7.25 (2H, d, J=8.2 Hz), 7.60 (1H, s).

ESI-MS (m/e): 577.4 [M+H]+

### Example 103

### Synthesis of N-[2-({8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]piperidine-1-carboxyamide

The title compound was obtained as a white solid by the method as in Example 52, using the 3-5 compound obtained in Reference Example 26.

The analytical data of the title compound are shown below.

1H-NMR (DMSO-D6) δ: 1.35-1.47 (16H, m), 3.33-3.36 (5H, brm), 4.15 (1H, d, J=14.1 Hz), 4.37 (3H, t, J=18 Hz), 6.34 (1H, s), 7.22-7.35 (7H, m).

ESI-MS (m/e): 580.4 [M+H]+

### Example 104

### Synthesis of 6-chloro-N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]pyridine-2-carboxyamide

The title compound was obtained as a white solid by the method as in Reference Example 3, using the compound obtained in Reference Example 26 and 6-chloropyridine-2-carboxylic acid.

The analytical data of the title compound are shown below.

1H-NMR (DMSO-D6) δ: 1.52 (6H, d, J=5.9 Hz), 3.08 (1H, dd, J=9.4, 7.4 Hz), 3.33-3.35 (4H, m), 4.13 (1H, d, J=14.1 Hz), 4.38 (1H, d, J=14.5 Hz), 4.44 (1H, dt, J=14.2, 5.9 Hz), 7.11 (1H, td, J=8.5, 2.7 Hz), 7.29 (3H, t, J=8 Hz), 7.39 (3H, dt, J=14.1, 5.6 Hz), 7.73 (1H, t, J=3.9 Hz), 7.95 (1H, dd, J=7.6, 1 Hz), 8.05 (1H, t, J=7.8 Hz), 8.14 (1H, d, J=8.2 Hz), 8.70 (1H, s).

ESI-MS (m/e): 608.4 [M+H]+

### Example 105

### Synthesis of 3-fluoro-N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]pyridine-2-carboxyamide

The title compound was obtained as a white solid by the method as in Reference Example 3, using the compound obtained in Reference Example 26 and 2-fluoropyridine-2-carboxylic acid.

The analytical data of the title compound are shown below.

1H-NMR (DMSO-D6) δ: 1.43 (6H, d, J=15.6 Hz), 3.20 (2H, dt, J=18.0, 8.4 Hz), 3.32 (3H, d, J=5.9 Hz), 4.11-4.44 (3H, m), 7.11 (1H, td, J=8.5, 3 Hz), 7.30 (3H, q, J=4.2 Hz), 7.38 (3H, dt, J=12.9, 5.3 Hz), 7.54 (1H, dd, J=8.2, 4.7 Hz), 7.63 (1H, d, J=7.8 Hz), 7.98 (1H, dd, J=8.2, 1.6 Hz), 8.52 (1H, dd, J=4.5, 1.4 Hz), 8.73 (1H, s).

ESI-MS (m/e): 592.4 [M+H]+

### Example 106

### Synthesis of N-[2-({(3R)-8-chloro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]-4-fluorobenzamide

The title compound was obtained as a white solid by the method as in Example 43, using the compound obtained in Reference Example 28.

The analytical data of the title compound are shown below.

1H-NMR (CDCl3) δ: 1.64 (3H, s), 1.64 (3H, s), 3.10-3.16 (1H, m), 3.42 (3H, s), 3.52-3.56 (1H, m), 4.05 (1H, d, J=14.1 Hz), 4.34 (1H, d, J=14.1 Hz), 4.59-4.65 (1H, m), 6.79 (1H, s), 7.00-7.20 (8H, m), 7.24-7.26 (2H, m), 7.72-7.75 (2H, m).

ESI-MS (m/e): 607 [M+H]+

### Example 107

### Synthesis of N-[2-({(3R)-8-chloro-1-methyl-2-oxo-5-[4-(trifluoromethyl)benzyl]-2,3,4,5-tetrahydro-1H-1,5-benzodiazepin-3-yl}amino)-1,1-dimethyl-2-oxoethyl]-5-fluoropyridine-2-carboxyamide

The title compound was obtained as a white solid by the method as in Example 44, using the compound obtained in Reference Example 28.

The analytical data of the title compound are shown below.

1H-NMR (DMSO-D6) δ: 1.51 (6H, s), 3.12 (1H, dd, J=9.0, 7 Hz), 3.25-3.38 (1H, m), 3.30 (3H, s), 4.20 (1H, d, J=14.9 Hz), 4.38-4.52 (2H, m), 7.23-7.32 (2H, m), 7.45 (2H, d, J=8.2 Hz), 7.56 (1H, d, J=2.3 Hz), 7.67 (2H, d, J=8.2 Hz), 7.88 (1H, td, J=8.6, 3.1 Hz), 7.99-8.09 (2H, m), 8.61 (1H, d, J=2.7 Hz), 8.77 (1H, s).

ESI-MS (m/e): 592 [M+H]+

### Example 108

### Synthesis of N-{1-[({(3R)-7-chloro-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide

The title compound was obtained as a white solid by the method as in Example 67, using the compound obtained in Reference Example 23.

The analytical data of the title compound are shown below.

¹H-NMR (CDCl₃) δ: 1.06-1.09 (2H, m), 1.53-1.60 (2H, m), 3.07 (1H, dd, J=12.0, 9.4 Hz), 3.32 (3H, s), 3.44 (1H, d d, J=9.4, 7 Hz), 3.96 (1H, d, J=13.9 Hz), 4.30 (1H, d, J=13.9 Hz), 4.57-4.60 (1H, m), 6.61 (1H, s), 7.04-7.05 (3H, m), 7.10 (2H, d, J=7.8 Hz), 7.21 (2H, d, J=7.8 Hz), 7.30 (1H, d, J=6.3 Hz), 7.39-7.43 (2H, m), 7.48-7.52 (1H, m), 7.73 (2H, d, J=7 Hz).

ESI-MS (m/e): 587 [M+H]+

### Example 109

### Synthesis of N-{1-[({(3R)-5-[3,5-bis(trifluoromethyl)benzyl]-7-chloro-1-methyl-2-oxo-2,3,4,5-tetrahydro-1H-1,5-benzodiazepin-3-yl}amino)carbonyl]cyclopropyl}benzamide

The title compound was obtained as a white solid by the method as in Example 64, using the compound obtained in Reference Example 23.

The analytical data of the title compound are shown below.

¹H-NMR (CDCl₃) δ: 1.07-1.19 (2H, m), 1.56-1.70 (2H, m), 3.19 (1H, t, J=12.0, 9.8 Hz), 3.36 (3H, s), 3.68 (1H, dd, J=9.8, 6.3 Hz), 4.14 (1H, d, J=14.9 Hz), 4.50 (1H, d, J=14.9 Hz), 4.64 (1H, dt, J=12.0, 6.3 Hz), 6.71 (1H, s), 7.02 (1H, s), 7.09 (2H, s), 7.41 (1H, d, J=6.6 Hz), 7.40-7.48 (2H, m), 7.53-7.56 (1H, m), 7.65 (2H, s), 7.71 (1H, s), 7.79 (2H, dJ=8 Hz).

ESI-MS (m/e): 639 [M+H]+

### Reference Example 1

### Synthesis of tert-butyl[(3R)-8-fluoro-1-methyl-2-oxo-2,3,4,5-tetrahydro-1H-1,5-benzodiazepin-3-yl]carbamate

### Step 1 Synthesis of N-(tert-butoxycarbonyl)-3-[(4-fluoro-2-nitrophenyl)amino]-D-alanine

In ethanol was dissolved 2,5-fluoronitrobenzene, and to the solution were added 3-amino-N-(tert-butoxycarbonyl)-D-alanine and potassium carbonate. The reaction liquid was stirred overnight at 80° C. The reaction liquid was cooled to room temperature, followed by removing ethanol under reduced pressure and adding water to the residue. The resultant aqueous solution was washed with ether twice, and the aqueous phase was adjusted to pH 4.3 with 1M hydrochloric acid. Two extractions were carried out with ethyl acetate, and the organic phase was washed with a saturated saline solution, dried over anhydrous sodium sulfate and thereafter concentrated under reduced pressure to yield the crude title compound as an orange solid.

ESI-MS (m/e): 344 [M+H]+

### Step 2 Synthesis of 3-[(2-amino-4-fluorophenyl)amino]-N-(tert-butoxycarbonyl)-D-alanine

The compound obtained in (Step 1) was dissolved in methanol, 10% Pd/C (3 g) was added to the solution, and the inside of a reaction vessel was substituted with hydrogen. The reaction liquid was stirred at room temperature for 8 hours, followed by Celite-filtering the reaction liquid and washing Celite with chloroform. The filtrate was removed under reduced pressure to yield the crude title compound as a brown solid.

ESI-MS (m/e): 344 [M+H]+

### Step 3 Synthesis of tert-butyl[(3R)-8-fluoro-2-oxo-2,3,4,5-tetrahydro-1H-1,5-benzodiazepin-3-yl]carbamate

In DMF was dissolved the compound obtained in (Step 2), and to the solution were added triethylamine, HOBt and WSC sequentially. The mixture was stirred overnight at room temperature. A saturated aqueous ammonium chloride solution was added to the reaction liquid under ice-cooling, and the mixture was extracted with ethyl acetate. The organic phase was washed with water and a saturated saline solution and dried over anhydrous sodium sulfate, and the solvent was distilled off under reduced pressure. The resultant crude product was purified from hexane-ethyl acetate by crystallization to yield the title compound as a light brown solid.

¹H-NMR (CDCl₃) δ: 1.43 (9H, s), 3.40 (1H, t, J=10.8 Hz), 3.67-3.73 (1H, m), 3.89-3.91 (1H, m), 4.50-4.56 (1H, m), 5.63 (1H, d, J=5.9 Hz), 6.65 (1H, dd, J=8.8, 2.5 Hz), 6.71-6.80 (2H, m), 7.66 (1H, s).

### Step 4 Synthesis of tert-butyl[(3R)-8-fluoro-1-methyl-2-oxo-2,3,4,5-tetrahydro-1H-1,5-benzodiazepin-3-yl]carbamate

The compound obtained in (Step 3) was dissolved in DMF under nitrogen atmosphere, and methyl iodide was added to the solution. The reaction liquid was cooled to −20° C., and sodium t-pentoxide was separately added to the reaction liquid. The temperature of the reaction liquid was gradually increased to room temperature, and the reaction liquid was stirred at the room temperature for 6 hours. The reaction liquid was cooled to 0° C., a saturated aqueous ammonium chloride solution was then added to the reaction liquid, and the mixture was extracted with ethyl acetate. The organic phase was washed with water and a saturated saline solution and dried over anhydrous sodium sulfate, and the solvent was distilled off under reduced pressure. The resultant residue was purified by silica gel chromatography to yield the title compound as a white solid.

¹H-NMR (CDCl₃) δ: 1.42 (9H, s), 3.29-3.37 (2H, m), 3.38 (3H, s), 3.91 (1H, t, J=7.6 Hz), 4.52 (1H, dt, J=13.2, 5.8 Hz), 5.56 (1H, d, J=7.0 Hz), 6.79-6.89 (3H, m).

ESI-MS (m/e): 310 [M+H]+

### Reference Example 2

### Synthesis of (3R)-3-amino-8-fluoro-1-methyl-1,3,4,5-tetrahydro-2H-1,5-benzodiazepin-2-on

The compound obtained in Reference Example 1 was added to chloroform, and trifluoroacetic acid was added to the solution under ice-cooling. The reaction liquid was stirred at room temperature for 1 hour, followed by removing the solvent under reduced pressure. To the resultant residue was added 1M aqueous sodium hydroxide under ice-cooling to be basified, and the mixture was extracted with chloroform-methanol mixed solvent. The extract was dried over anhydrous sodium sulfate and thereafter filtered, and the solvent was distilled off under reduced pressure to yield the crude title compound.

¹H-NMR (CDCl₃) δ: 3.26 (1H, dd, J=11.7, 9.4 Hz), 3.35 (3H, s), 3.60 (1H, dd, J=11.5, 6.8 Hz), 3.71-3.75 (1H, m), 6.76-6.87 (3.0H, m).

### Reference Example 3

### Synthesis of 4-fluoro-N-(2-{[(3R)-8-fluoro-1-methyl-2-oxo-2,3,4,5-tetrahydro-1H-1,5-benzodiazepin-3-yl]amino}-1,1-dimethyl-2-oxoethyl)benzamide

In DMF was dissolved the compound obtained in Reference Example 2, and to the solution were added the compound obtained in Reference Example 39, triethylamine, HOBt and WSC. The reaction liquid was stirred overnight at room temperature and then poured into water. The mixture was extracted with ethyl acetate, and the extract was washed with water and a saturated saline solution. The organic phase was dried over sodium sulfate and thereafter filtered, and the solvent was distilled off under reduced pressure. The resultant residue was purified by silica gel chromatography (chloroform-20% methanol-chloroform mixed solvent, 100:0-85:15) to yield the title compound as a pale yellow solid.

¹H-NMR (CDCl₃) δ: 1.67 (6H, s), 3.33 (1H, dd, J=12.4, 9.4 Hz), 3.39 (3H, s), 4.03 (1H, dd, J=9.4, 6.6 Hz), 4.73 (1 H, dt, J=12.4, 6.6 Hz), 6.82-6.90 (4H, m), 7.07-7.12 (3H, m), 7.78 (2H, dd, J=8.0, 4.0 Hz).

### Reference Example 4

### Synthesis of N-(2-{[(3R)-8-fluoro-1-methyl-2-oxo-2,3,4,5-tetrahydro-1H-1,5-benzodiazepin-3-yl]amino}-1,1-dimethyl-2-oxoethyl)benzamide

The title compound was obtained by the method as in Reference Example 3, using the compound obtained in Reference Example 35.

### Reference Example 5

### Synthesis of N-[1-({[(3R)-8-fluoro-1-methyl-2-oxo-2,3,4,5-tetrahydro-1H-1,5-benzodiazepin-3-yl]amino}carbonyl)cyclopropyl]benzamide

The title compound was obtained by the method as in Reference Example 3, using the compound obtained in Reference Example 34.

### Reference Example 6

### Synthesis of 5-fluoro-N-(2-{[(3R)-8-fluoro-1-methyl-2-oxo-2,3,4,5-tetrahydro-1H-1,5-benzodiazepin-3-yl]amino}-1,1-dimethyl-2-oxoethyl)pyridine-2-carboxyamide

The title compound was obtained by the method as in Reference Example 3, using the compound obtained in Reference Example 36.

### Reference Example 7

### Synthesis of 2-fluoro-N-(2-{[(3R)-8-fluoro-1-methyl-2-oxo-2,3,4,5-tetrahydro-1H-1,5-benzodiazepin-3-yl]amino}-1,1-dimethyl-2-oxoethyl)isonicotinamide

The title compound was obtained by the method as in Reference Example 3, using the compound obtained in Reference Example 37.

### Reference Example 8

### Synthesis of N-[(3R)-8-fluoro-1-methyl-2-oxo-2,3,4,5-tetrahydro-1H-1,5-benzodiazepin-3-yl]-2-methyl-N²-{[1-(trifluoromethyl)cyclopropyl]carbonyl}alaninamide

The title compound was obtained by the method as in Reference Example 3, using the compound obtained in Reference Example 38.

### Reference Example 9

### Synthesis of tert-butyl[(3R)-2-oxo-2,3,4,5-tetrahydro-1H-1,5-benzodiazepin-3-yl]carbamate

The title compound was obtained by the method as in Reference Example 1, using 1-fluoro-2-nitrobenzene.

### Reference Example 10

### Synthesis of tert-butyl[(3R)-1-methyl-2-oxo-2,3,4,5-tetrahydro-1H-1,5-benzodiazepin-3-yl]carbamate

The title compound was obtained by the method as in Reference Example 1 (Step 4), using the compound obtained in Reference Example 9.

### Reference Example 11

### Synthesis of N-(1,1-dimethyl-2-{[(3R)-1-methyl-2-oxo-2,3,4,5-tetrahydro-1H-1,5-benzodiazepin-3-yl]amino}-2-oxoethyl)benzamide

The title compound was obtained by the method as in Reference Examples 2 and 4, using the compound obtained in Reference Example 10.

### Reference Example 12

### Synthesis of N-(1,1-dimethyl-2-{[(3R)-1-methyl-2-oxo-2,3,4,5-tetrahydro-1H-1,5-benzodiazepin-3-yl]amino}-2-oxoethyl)-4-fluorobenzamide

The title compound was obtained by the method as in Reference Examples 2 and 3, using the compound obtained in Reference Example 10.

### Reference Example 13

### Synthesis of N-[1-({[(3R)-1-methyl-2-oxo-2,3,4,5-tetrahydro-1H-1,5-benzodiazepin-3-yl]amino}carbonyl)cyclopropyl]benzamide

The title compound was obtained by the method as in Reference Examples 2 and 5, using the compound obtained in Reference Example 10.

### Reference Example 14

### Synthesis of 4-fluoro-N-[1-({[(3R)-1-methyl-2-oxo-2,3,4,5-tetrahydro-1H-1,5-benzodiazepin-3-yl]amino}carbonyl)cyclopropyl]benzamide

The title compound was obtained by the method as in Reference Examples 2 and 3, using the compounds obtained in Reference Examples 10 and 40.

### Reference Example 15

### Synthesis of N-(1,1-dimethyl-2-oxo-2-{[(3R)-2-oxo-2,3,4,5-tetrahydro-1H-1,5-benzodiazepin-3-yl]amino}ethyl)benzamide

The title compound was obtained by the method as in Reference Examples 2 and 4, using the compound obtained in Reference Example 9.

### Reference Example 16

### Synthesis of tert-butyl[(3R)-8-chloro-1-methyl-2-oxo-2,3,4,5-tetrahydro-1H-1,5-benzodiazepin-3-yl]carbamate

The title compound was obtained by the method as in Reference Example 1, using 4-chloro-1-fluoro-2-nitrobenzene.

### Reference Example 17

### Synthesis of N-[1-({[(3R)-8-chloro-1-methyl-2-oxo-2,3,4,5-tetrahydro-1H-1,5-benzodiazepin-3-yl]amino}carbonyl)cyclopropyl]benzamide

The title compound was obtained by the method as in Reference Examples 2 and 5, using the compound obtained in Reference Example 16.

### Reference Example 18

### Synthesis of N-(2-{[(3R)-8-chloro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained by the method as in Reference Examples 2 and 3, using the compound obtained in Reference Example 16.

### Reference Example 19

### Synthesis of N-(2-{[(3R)-8-chloro-1-methyl-2-oxo-2,3,4,5-tetrahydro-1H-1,5-benzodiazepin-3-yl]amino}-1,1-dimethyl-2-oxoethyl)-5-fluoropyridine-2-carboxyamide

The title compound was obtained by the method as in Reference Examples 2 and 6, using the compound obtained in Reference Example 16.

### Reference Example 20

### Synthesis of N-(2-{[(3R)-8-chloro-1-methyl-2-oxo-2,3,4,5-tetrahydro-1H-1,5-benzodiazepin-3-yl]amino}-1,1-dimethyl-2-oxoethyl)-2-fluoroisonicotinamide

The title compound was obtained by the method as in Reference Examples 2 and 7, using the compound obtained in Reference Example 16.

### Reference Example 21

### Synthesis of N-[(3R)-8-chloro-1-methyl-2-oxo-2,3,4,5-tetrahydro-1H-1,5-benzodiazepin-3-yl]-2-methyl-N²-{[1-(trifluoromethyl) cyclopropyl]carbonyl}alaninamide

The title compound was obtained by the method as in Reference Examples 2 and 8, using the compound obtained in Reference Example 16.

### Reference Example 22

### Synthesis of tert-butyl[(3R)-7-chloro-1-methyl-2-oxo-2,3,4,5-tetrahydro-1H-1,5-benzodiazepin-3-yl]carbamate

The title compound was obtained by the method as in Reference Example 1, using 4-chloro-2-fluoro-1-nitrobenzene.

### Reference Example 23

### Synthesis of N-[1-({[(3R)-7-chloro-1-methyl-2-oxo-2,3,4,5-tetrahydro-1H-1,5-benzodiazepin-3-yl]amino}carbonyl)cyclopropyl]benzamide

The title compound was obtained by the method as in Reference Examples 2 and 5, using the compound obtained in Reference Example 22.

### Reference Example 24

### Synthesis of tert-butyl[1-({[(3R)-1-methyl-2-oxo-2,3,4,5-tetrahydro-1H-1,5-benzodiazepin-3-yl]amino}carbonyl)cyclopropyl]carbamate

The title compound was obtained by the method as in Reference Examples 2 and 3, using the compound obtained in Reference Example 10 and 1-[(tert-butoxycarbonyl)amino]cyclopropanecarboxylic acid.

### Reference Example 25

### Synthesis of 1-amino-N-{(3R)-1-methyl-2-oxo-5-[4-(trifluoromethoxy)benzyl]-2,3,4,5-tetrahydro-1H-1,5-benzodiazepin-3-yl}cyclopropanecarboxyamide

A solution of compound obtained in Example 104 in chloroform was ice-cooled, followed by adding trifluoroacetic acid and stirring the mixture for 4 hours while gradually bringing it back to room temperature. The solvent was distilled off, followed by diluting the residue with ethyl acetate, adding saturated sodium bicarbonate water and extracting the solution with ethyl acetate. The organic layer was washed with a saturated saline solution and thereafter dried over anhydrous magnesium sulfate. After filtration, the solvent was distilled off to yield the title compound as yellow oil. The compound was used in the subsequent step without being purified.

### Reference Example 26

### Synthesis of N-{(3R)-8-fluoro-1-methyl-2-oxo-5-[4-(trifluoromethoxy)benzyl]-2,3,4,5-tetrahydro-1H-1,5-benzodiazepin-3-yl}-2-methylalaninamide

The title compound was obtained by the methods as in Reference Example 24 using a compound obtained in Reference Example 1, subsequently as in Example 104 using N-(tert-butoxycarbonyl)-2-methylalanine and subsequently as in Reference Example 25.

### Reference Example 27

### Synthesis of 1-amino-N-{(3R)-8-chloro-1-methyl-2-oxo-5-[4-(trifluoromethoxy)benzyl]-2,3,4,5-tetrahydro-1H-1,5-benzodiazepin-3-yl}cyclopropanecarboxyamide

The title compound was obtained by the methods as in Reference Example 24 using a compound obtained in Reference Example 16, as in Example 104 and subsequently as in Reference Example 25.

### Reference Example 28

### Synthesis of N-{(3R)-8-chloro-1-methyl-2-oxo-5-[4-(trifluoromethoxy)benzyl]-2,3,4,5-tetrahydro-1H-1,5-benzodiazepin-3-yl}-2-methylalaninamide

The title compound was obtained by the method as in Reference Example 26, using the compound obtained in Reference Example 16.

### Reference Example 29

### Synthesis of tert-butyl[(3R)-5-(3,5-dichlorophenyl)-8-fluoro-1-methyl-2-oxo-2,3,4,5-tetrahydro-1H-1,5-benzodiazepin-3-yl]carbamate

The title compound was obtained by the method as in Example 3, using the compound obtained in Reference Example 1.

### Reference Example 30

### Synthesis of N-[(3R)-5-(3,5-dichlorophenyl)-8-fluoro-1-methyl-2-oxo-2,3,4,5-tetrahydro-1H-1,5-benzodiazepin-3-yl]-2-methylalaninamide

The title compound was obtained by the method as in Example 26, using the compound obtained in Reference Example 29.

### Reference Example 31

### Synthesis of N-{(3R)-8-fluoro-1-methyl-2-oxo-5-[4-(trifluoromethoxy)phenyl]-2,3,4,5-tetrahydro-1H-1,5-benzodiazepin-3-yl}-2-methylalaninamide

The title compound was obtained by the methods as in Example 1 and subsequently as in Example 26, using the compound obtained in Reference Example 1.

### Reference Example 32

### Synthesis of 4-fluoro-N-(2-{[(3R)-7-fluoro-1-methyl-2-oxo-2,3,4,5-tetrahydro-1H-1,5-benzodiazepin-3-yl]amino}-1,1-dimethyl-2-oxoethyl)benzamide

The title compound was obtained by the methods sequentially as in Reference Examples 1, 2 and 3 using 2,4-difluoronitrobenzene.

### Reference Example 33

### Synthesis of N-(2-{[(3R)-7,8-difluoro-1-methyl-2-oxo-2,3,4,5-tetrahydro-1H-1,5-benzodiazepin-3-yl]amino}-1,1-dimethyl-2-oxoethyl)-4-fluorobenzamide

The title compound was obtained by the methods sequentially as in Reference Examples 1, 2 and 3 using 2,4,5-trifluoronitrobenzene.

### Reference Example 34

### Synthesis of 1-(benzoylamino)cyclopropanecarboxylic acid

### Step 1

### Synthesis of ethyl 1-(benzoylamino)cyclopropanecarboxylic acid

Benzoic acid, 1-hydroxybenzotriazole monohydrate, 1-(3-dimethylaminopropyl)-3-ethylcarbodiimide hydrochloride and triethylamine were sequentially added to a mixed solution of 1-aminocyclopropane-1-carboxylic acid ethyl ester hydrochloride in N,N-dimethylformamide and water, and the mixture was stirred overnight at room temperature. A saturated aqueous ammonium chloride solution and water were added to the reaction solution, the resultant precipitate was obtained by filtration, and the obtained precipitate was dried to yield ethyl 1-(benzoylamino)cyclopropanecarboxylate as a white powder.

### Step 2

### Synthesis of 1-(benzoylamino)cyclopropanecarboxylic acid

To a solution of ethyl 1-(benzoylamino)cyclopropanecarboxylate in ethanol was added 5N aqueous sodium hydroxide, and the mixture was stirred at room temperature for 6 hours. A 5N aqueous hydrochloric acid solution and water were added, the resultant precipitate was obtained by filtration, and the obtained precipitate was dried to yield ethyl 1-(benzoylamino)cyclopropanecarboxylic acid as a white powder.

### Reference Example 35

### Synthesis of N-benzoyl-2-methylalanine

The title compound was obtained by the method as in Reference Example 34, using 2-methyl alanine ethyl ester hydrochloride.

### Reference Example 36

### Synthesis of N-[(5-fluoropyridine-2-yl)]carbonyl]-2-methylalanine

The title compound was obtained by the method as in Reference Example 35, using 5-fluoropyridine-2-carboxylic acid.

### Reference Example 37

### Synthesis of N-(2-fluoroisonicotinoyl)-2-methylalanine

The title compound was obtained by the method as in Reference Example 35, using 5-fluoroisonicotinic acid.

### Reference Example 38

### Synthesis of 1-methyl-N-{[1-(trifluoromethyl)cyclopropyl]carbonyl}alanine

The title compound was obtained by the method as in Reference Example 35, using 1-(trifluoromethyl)cyclopropanecarboxylic acid.

### Reference Example 39

### Synthesis of N-(4-fluorobenzoyl)-2-methylalanine

The title compound was obtained by the method as in Reference Example 35, using 4-fluorobenzoic acid.

### Reference Example 40

### Synthesis of 1-[(4-fluorobenzoyl)amino]cyclopropanecarboxylic acid

The title compound was obtained by the method as in Reference Example 34, using 4-fluorobenzoic acid.

### Reference Example 41

### Synthesis of N-(4-fluorobenzoyl)glycine

The title compound was obtained by the method as in Reference Example 39, using glycine ethyl ester hydrochloride.

### Reference Example 42

### Synthesis of N-(4-fluorobenzoyl)-L-alanine

The title compound was obtained by the method as in Reference Example 39, using L-alanine ethyl ester hydrochloride.

The usefulness of the compound represented by the formula (I) as a medicament is proved, for example, in the assay described below.

### Cloning of Human DGAT1 Gene and Expression in Yeast

Human DGAT1 genes were amplified by PCR using primers described below from human cDNA library (Clontech).

DGAT1F: 5′-ATGGGCGACCGCGGCAGCTC-3′
DGAT1R: 5′-CAGGCCTCTGCCGCTGGGGCCTC-3′

The amplified human DGAT1 genes were introduced into a yeast expression vector pPICZA (Invitrogen). The resultant expression plasmid was introduced into an yeast (Pichia pastris) by electroporation to produce a recombinant yeast. The recombinant yeast was cultured in the presence of 0.5% methanol for 72 hours, and the cells were crushed using glass beads in 10 mM Tris pH 7.5, 250 mM sucrose and 1 mM EDTA, followed by adjusting the membrane fraction by centrifugation to use the adjusted membrane fraction as an enzyme source.

### DGAT1 Inhibitory Activity Test

To the reaction liquid having the following composition: 100 mM Tris pH 7.5, 100 mM MgCl₂, 100 mM sucrose, 40 μM Dioelin, 15 μM [¹⁴C]-oleoyl-CoA, 0.25 μg of test substance, DGAT1-expressed yeast membrane fraction, was added, and the mixture having a volume of 100 μl was incubated at room temperature for 30 minutes. To the reaction liquid, 100 μl of 2-propanol/heptan/H₂O (80/20/2) was added, the mixture was stirred well, followed by adding 200 of heptane and further stirring the mixture. After centrifugation, the heptane layer was collected, ethanol/0.1N NaOH/H₂O (50:5:45) was added, the mixture was thus stirred, followed by recentrifuging the mixture and collecting the heptane layer. After exsiccation of the resultant heptane layer, 100 μl of Microscint 0 (PerkinElmer) was added, and the radioactivity was measured with a liquid scintillation counter. The inhibitory activity was calculated from the following formula:

Inhibition rate=100−(radioactivity in case of addition of test compound−background)/(radioactivity in case of addition of no test compound−background)×100

wherein the background means the radioactivity in case of addition of no membrane fraction.

The DGAT1 inhibitory activity of the compound according to an embodiment of the present invention by the aforementioned method is shown below.

TABLE 5

| | DGAT1 |
|---|---|
| | Inhibitory |
| Example | Activity |
| Number | IC₅₀ (nM) |
| 1 | 166 |
| 12 | 192 |
| 19 | 24.1 |
| 27 | 16.3 |
| 45 | 29.2 |
| 47 | 61.3 |
| 52 | 91.1 |
| 59 | 40.2 |
| 64 | 47.3 |
| 84 | 140 |
| 92 | 20.8 |
| 98 | 1344 |
| 100 | 2233 |

1-11. (canceled)

12. A compound represented by formula (I): 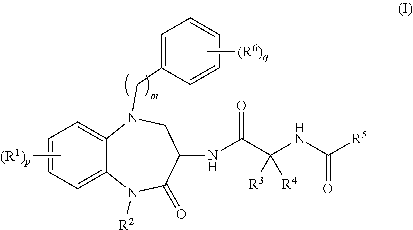
wherein R¹ each independently represents a hydrogen or halogen atom;
R² represents a hydrogen atom or C₁-C₆alkyl;
R³ and R⁴ each independently represent C₁-C₆alkyl or represent C₃₋₇ cycloalkyl formed by R³ and R⁴ together with the carbon atom to which they are bound;
R⁵ is a group selected from the group consisting of:
(1) phenyl, unsubstituted or substituted with 1 to 3 identical or different groups selected from the group consisting of halogen atoms and lower alkoxy which may be substituted with 1 to 3 identical or different halogen atoms;
(2) heteroaryl selected from the group consisting of pyridinyl, pyrazinyl, imidazolyl, thiazolyl, thienyl and oxazolyl, wherein the heteroaryl is unsubstituted or substituted with 1 to 3 identical or different groups selected from the group consisting of halogen atoms and C₁-C₆alkyl which is unsubstituted or substituted with 1 to 3 identical or different halogen atoms;
(3) —O—C₃₋₆ branched C₁-C₆alkyl, which is unsubstituted or substituted with 1 to 3 identical or different halogen atoms;
(4) C₃₋₇ cycloalkyl, which is unsubstituted or substituted with trifluoromethyl; and
(5) —N—(H)—C₃₋₆ branched C₁-C₆alkyl or —N(R⁷)R⁸, wherein N, R⁷ and R⁸ together form a 5-7 membered ring;
R⁶ each independently represents a group selected from the group consisting of a hydrogen atom, C₁-C₆alkyl, lower alkoxy, a halogen atom, cyano and lower alkoxycarbonylmethyl;
m is an integer from 0 to 2;
p is an integer from 1 to 4; and
q is an integer from 1 to 5,
or a pharmaceutically acceptable salt thereof.

13. The compound according to claim 12, wherein R² is methyl, or a pharmaceutically acceptable salt thereof.

14. The compound according to claim 13, wherein m is 0 or 1; and both R³ and R⁴ represent methyl, or R³ and R⁴ represent cyclopropyl formed by R³ and R⁴ together with the carbon atom to which they are bound, or a pharmaceutically acceptable salt thereof.

15. The compound according to claim 14, wherein R⁵ is a group selected from the group consisting of:
phenyl, which is unsubstituted or substituted with 1 or 2 identical or different groups selected from the group consisting of fluorine, chlorine, difluoromethoxy and trifluoromethoxy;
pyridinyl, pyrazinyl, imidazolyl, thiazolyl, thienyl or oxazolyl, wherein the pyridinyl, pyrazinyl, imidazolyl, thiazolyl, thienyl and oxazolyl is unsubstituted or substituted with 1 or 2 identical or different groups selected from the group consisting of fluorine, chlorine and methyl;
tert-butoxy, 1-ethylpropoxy or 1-(trifluoromethyl)cyclopropyl;
1-(trifluoromethyl)cyclopropyl or 4-trifluoromethylcyclohexyl; and
tert-butylamino or 1-piperidinyl,
or a pharmaceutically acceptable salt thereof.

16. The compound according to claim 14, wherein R⁵ is a group selected from the group consisting of phenyl, 4-chlorophenyl, 4-fluorophenyl, 3,4-difluorophenyl, 4-difluoromethoxyphenyl, 4-trifluoromethoxyphenyl, 5-fluoro-2-pyridinyl, 2-fluoro-4-pyridinyl, 3-chloro-2-thiazolyl, tert-butoxy, 1-(trifluoromethyl)cyclopropyl, 4-trifluoromethylcyclohexyl and tert-butylamino, or a pharmaceutically acceptable salt thereof.

17. The compound according to claim 14, wherein a group represented by formula (II): 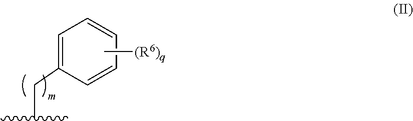
wherein [symbol]
represents a binding site, in the formula (I) is selected from the group consisting of 4-trifluoromethoxyphenyl, 3,5-dichlorophenyl, 2-chlorophenyl, 4-fluoro-3-trifluoromethylphenyl, 2-trifluoromethylphenyl, 2-fluorophenyl, 2-trifluoromethoxyphenyl, 4-trifluoromethoxyphenyl, 3,5-difluorophenyl, 3,4-dichlorophenyl, 3,5-dichlorophenyl, 4-chlorophenyl, 4-chloro-3-fluorophenyl, 3,4-difluorophenyl, 3-methylphenyl, 3-trifluoromethoxyphenyl, 3,5-bis(trifluoromethyl)phenyl and 3-chloro-5-fluorophenyl,
or a pharmaceutically acceptable salt thereof.

18. The compound according to claim 17, wherein R⁵ is a group selected from the group consisting of phenyl, 4-fluorophenyl, 4-difluoromethoxyphenyl, 4-trifluoromethoxyphenyl, 5-fluoro-2-pyridinyl, 1-(trifluoromethyl)cyclopropyl and tert-butoxy, or a pharmaceutically acceptable salt thereof.

19. The compound according to claim 12, wherein the compound represented by the formula (I) is:
4-fluoro-N-[2-({(3R)-8-fluoro-1-methyl-2-oxo-5-[4-(trifluoromethoxy)phenyl]-2,3,4,5-tetrahydro-1H-1,5-benzo diazepin-3-yl}amino)-1,1-dimethyl-2-oxoethyl]benzamide,
N-(2-{[(3R)-5-(3,5-difluorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,4-difluorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,4-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
4-fluoro-N-(2-{[(3R)-8-fluoro-1-methyl-5-(3-methylphenyl)-2-oxo-2,3,4,5-tetrahydro-1H-1,5-benzodiazepin-3-yl]amino}-1,1-dimethyl-2-oxoethyl)benzamide,
N-{1-[({(3R)-8-fluoro-1-methyl-2-oxo-5-[4-(trifluoromethoxy)phenyl]-2,3,4,5-tetrahydro-1H-1,5-benzo diazepin-3-yl}amino)carbonyl]cyclopropyl}benzamide,
N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide,
N-[2-({(3R)-8-fluoro-1-methyl-2-oxo-5-[3-(trifluoromethoxy)phenyl]-2,3,4,5-tetrahydro-1H-1,5-benzodiazepin-3-yl}amino)-1,1-dimethyl-2-oxoethyl]benzamide,
N-[2-({(3R)-5-[3,5-bis(trifluoromethyl)phenyl]-8-fluoro-1-methyl-2-oxo-2,3,4,5-tetrahydro-1H-1,5-benzodiazepin-3-yl}amino)-1,1-dimethyl-2-oxoethyl]-5-fluoropyridin-2-carboxyamide,
N-(2-{[(3R)-5-(3,5-difluorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-5-(3-chloro-5-fluorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-2-fluoroisonicotinamide,
N-(2-{[(3R)-8-chloro-5-(3,5-dichlorophenyl)-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-8-chloro-5-(3,5-difluorophenyl)-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-8-chloro-5-(3,5-dichlorophenyl)-1-methyl-2-oxo-2,3,4,5-tetrahydro-1H-1,5-benzodiazepin-3-yl]amino}-1,1-dimethyl-2-oxoethyl)-2-fluoroisonicotinamide,
N-{(3R)-5-[3,5-bis(trifluoromethyl)phenyl]-8-fluoro-1-methyl-2-oxo-2,3,4,5-tetrahydro-1H-1,5-benzodiazepin-3-yl}-2-methyl-N2-{[1-(trifluoromethyl)cyclopropyl]carbonyl}alaninamide,
N-[(3R)-8-chloro-5-(3,5-dichlorophenyl)-1-methyl-2-oxo-2,3,4,5-tetrahydro-1H-1,5-benzodiazepin-3-yl]-2-methyl-N2-{[1-(trifluoromethyl)cyclopropyl]carbonyl}alaninamide,
N-[(3R)-8-chloro-5-(3,5-difluorophenyl)-1-methyl-2-oxo-2,3,4,5-tetrahydro-1H-1,5-benzodiazepin-3-yl]-2-methyl-N2-{[1-(trifluoromethyl)cyclopropyl]carbonyl}alaninamide,
N-[2-({(3S))-5-[3,5-bis(trifluoromethyl)phenyl]-1-methyl-2-oxo-2,3,4,5-tetrahydro-1H-1,5-benzodiazepin-3-yl}amino)-1,1-dimethyl-2-oxoethyl]-4-fluorobenzamide,
N-[1,1-dimethyl-2-({(3R)-1-methyl-2-oxo-5-[4-(trifluoromethoxy)phenyl]-2,3,4,5-tetrahydro-1H-1,5-benzodiazepin-3-yl}amino)-2-oxoethyl]-4-fluorobenzamide,
N-(2-{[(3R)-5-(3,5-dichlorophenyl)-1-methyl-2-oxo-2,3,4,5-tetrahydro-1H-1,5-benzodiazepin-3-yl]amino}-1,1-dimethyl-2-oxoethyl)-4-fluorobenzamide,
N-{1-[({(3R)-8-chloro-1-methyl-2-oxo-5-[4-(trifluoromethoxy)phenyl]-2,3,4,5-tetrahydro-1H-1,5-benzodiazepin-3-yl}amino)carbonyl]cyclopropyl}benzamide,
N-(2-{[(3R)-8-chloro-5-(3,5-difluorophenyl)-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-8-chloro-5-(3,4-dichlorophenyl)-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-5-fluoropyridin-2-carboxyamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-1,3-thiazol-2-carboxyamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-(difluoromethoxy)benzamide,
N²-[(tert-butylamino)carbonyl]-N-[(3R)-5-(3,5-dichlorophenyl)-8-fluoro-1-methyl-2-oxo-2,3,4,5-tetrahydro-1H-1,5-benzodiazepin-3-yl]-2-methylalanineamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-4-(trifluoromethoxy)benzamide,
N-(2-{[(3R)-5-(3,5-dichlorophenyl)-8-fluoro-1-methyl-2-oxo-2,3,4,5-tetrahydro-1H-1,5-benzodiazepin-3-yl]amino}-1,1-dimethyl-2-oxoethyl)-3,4-difluorobenzamide,
4-(difluoromethoxy)-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]benzamide,
3-chloro-N-[2-({(3R)-8-fluoro-1-methyl-2-oxo-5-[4-(trifluoromethoxy)phenyl]-2,3,4,5-tetrahydro-1H-1,5-benzodiazepin-3-yl}amino)-1,1-dimethyl-2-oxoethyl]thiophene-2-carboxyamide,
N-(2-{[(3R)-5-(3,5-dichlorophenyl)-7-fluoro-1-methyl-2-oxo-2,3,4,5-tetrahydro-1H-1,5-benzodiazepin-3-yl]amino}-1,1-dimethyl-2-oxoethyl)-4-fluorobenzamide,
N-(2-{[(3R)-5-(3,5-dichlorophenyl)-7,8-difluoro-1-methyl-2-oxo-2,3,4,5-tetrahydro-1H-1,5-benzodiazepin-3-yl]amino}-1,1-dimethyl-2-oxoethyl)-4-fluorobenzamide,
N-{1-[({(3R)-5-[3,5-bis(trifluoromethyl)benzyl]-1-methyl-2-oxo-2,3,4,5-tetrahydro-1H-1,5-benzodiazepin-3-yl}amino)carbonyl]cyclopropyl}benzamide,
N-[1-({[(3R)-5-(3,5-dichlorobenzyl)-1-methyl-2-oxo-2,3,4,5-tetrahydro-1H-1,5-benzodiazepin-3-yl]amino}carbonyl)cyclopropyl]benzamide,
N-{1-[({(3R)-1-methyl-2-oxo-5-[4-(trifluoromethoxy)benzyl]-2,3,4,5-tetrahydro-1H-1,5-benzodiazepin-3-yl]amino}carbonyl)cyclopropyl}benzamide,
N-[1-({[(3R)-5-(4-chlorobenzyl)-8-fluoro-1-methyl-2-oxo-2,3,4,5-tetrahydro-1H-1,5-benzodiazepin-3-yl]amino}carbonyl)cyclopropyl]benzamide,
N-(2-{[(3R)-5-(3,5-dichlorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
4-fluoro-N-[2-({(3R)-8-fluoro-5-[4-fluoro-3-(trifluoromethyl)benzyl]-1-methyl-2-oxo-2,3,4,5-tetrahydro-1H-1,5-benzodiazepin-3-yl}amino)-1,1-dimethyl-2-oxoethyl]benzamide,
N-(2-{[(3R)-5-(2-chlorobenzyl)-8-fluoro-1-methyl-2-oxo-2,3,4,5-tetrahydro-1H-1,5-benzodiazepin-3-yl]amino}-1,1-dimethyl-2-oxoethyl)-4-fluorobenzamide,
4-fluoro-N-[2-({(3R)-8-fluoro-1-methyl-2-oxo-5-[2-(trifluoromethyl)benzyl]-2,3,4,5-tetrahydro-1H-1,5-benzodiazepin-3-yl}amino)-1,1-dimethyl-2-oxoethyl]benzamide,
4-fluoro-N-(2-{[(3R)-8-fluoro-5-(2-fluorobenzyl)-1-methyl-2-oxo-2,3,4,5-tetrahydro-1H-1,5-benzo diazepin-3-yl]amino}-1,1-dimethyl-2-oxoethyl)benzamide,
N-[2-({(3R)-5-[2-(difluoromethoxy)benzyl]-8-fluoro-1-methyl-2-oxo-2,3,4,5-tetrahydro-1H-1,5-benzodiazepin-3-yl}amino)-1,1-dimethyl-2-oxoethyl]-4-fluorobenzamide,
N-(2-{[(3R)-5-(4-chloro-3-fluorobenzyl)-8-fluoro-1-methyl-2-oxo-2,3,4,5-tetrahydro-1H-1,5-benzo diazepin-3-yl]amino}-1,1-dimethyl-2-oxo ethyl)-4-fluorobenzamide,
tert-butyl{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}carbamate,
4-chloro-N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide,
N-{1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}pyridin-2-carboxyamide,
1-ethylpropyl {1-[({(3R)-1-methyl-2-oxo-5-[4-(trifluoromethoxy)benzyl]-2,3,4,5-tetrahydro-1H-1,5-benzodiazepin-3-yl}amino)carbonyl]cyclopropyl}carbamate,
N-[2-({(3R)-8-fluoro-1-methyl-2-oxo-5-[4-(trifluoromethoxy)benzyl]-2,3,4,5-tetrahydro-1H-1,5-benzodiazepin-3-yl}amino)-1,1-dimethyl-2-oxoethyl]-4-(trifluoromethyl)cyclohexanecarboxyamide, or
N-{1-[({(3R)-7-chloro-1-methyl-2-oxo-5-[4-(trifluoromethoxy)benzyl]-2,3,4,5-tetrahydro-1H-1,5-benzodiazepin-3-yl}amino)carbonyl]cyclopropyl}benzamide,
or a pharmaceutically acceptable salt thereof.

20. A pharmaceutical composition comprising a compound according to claim 12 and a pharmaceutically acceptable carrier.

21. (canceled)

22. A method for treating hyperlipidemia, diabetes and/or obesity, comprising administering an effective amount of a compound according to claim 12 to a patient in need thereof.


SEQUENCE LISTING (2 sequences)

SEQ ID NO: 1 - DNA, 20 bp, homo sapiens
atgggcgaccgcggcagctc

SEQ ID NO: 2 - DNA, 23 bp, homo sapiens
caggcctctgccgctggggcctc